(19) United States
(12) Patent Application Publication
(10) Pub. No.: US 2003/0096255 A1
(43) Pub. Date: May 22, 2003

(54) Methods and kits for analysis of chromosomal rearrangements associated with cancer

(76) Inventors: Carolyn A. Felix, Ardmore, PA (US); Douglas H. Jones, Cedar Rapids, IA (US); Eric Rappaport, Blackwood, NJ (US)

Correspondence Address: DANN DORFMAN HERRELL & SKILLMAN, SUITE 720, 1601 MARKET STREET, PHILADELPHIA, PA, 19103-2307, US

(21) Appl. No.: 10/118,783

(22) Filed: Apr. 9, 2002

Related U.S. Application Data
(63) Continuation-in-part of application No. 09/026,033, filed on Feb. 19, 1998, now Pat. No. 6,368,791.
(60) Provisional application No. 60/038,624, filed on Feb. 19, 1997.
(60) Provisional application No. 60/056,938, filed on Aug. 25, 1997.
(60) Provisional application No. 60/065,911, filed on Nov. 17, 1997.

(51) Int. Cl. C12Q 1/68; C12P 19/34

(52) U.S. Cl. 435/6; 435/91.2

(57) ABSTRACT

The invention relates to kits and methods for panhandle PCR amplification of a region of DNA having an unknown nucleotide sequence, wherein the region flanks a region of a cancer-associated gene having a known nucleotide sequence in a human patient. Amplification of an unknown region flanking a known region of a cancer-associated gene permits identification of a translocation partner of the gene or identification of a replicated sequence within the gene. The invention further relates to kits useful for performing the methods of the invention, to an isolated polynucleotide, and to primers derived from such an isolated polynucleotide.

### CROSS REFERENCE TO RELATED APPLICATIONS

This application is a continuation-in- part application of U.S. application Ser. No. 09/026,033, filed Feb. 19, 1998, which in turn claims priority to each of the following provisional patent applications: U.S. application Ser. No. 60/038,624, filed Feb. 19, 1997, U.S. application Ser. No. 60/056,938, filed Aug. 26, 1997, and U.S. application Ser. No. 60/065,911, filed Nov. 17, 1997. Each of the foregoing applications is incorporated by reference herein.

### STATEMENT REGARDING FEDERALLY SUPPORTED RESEARCH AND DEVELOPMENT

Pursuant to 35 U.S.C. §202(c), it is acknowledged that the U.S. Government has certain rights in the invention described herein, which was made in part with funds from the National Institutes of Health, USPHS Grant Numbers CA66140, CA80175, CA77683, CA85469.

### FIELD OF THE INVENTION

This invention relates to the field of cancer diagnosis and rational drug design. More specifically, the invention provides compositions and methods for the identifying and analyzing chromosomal rearrangements which are associated with cancer. The rearrangements or translocations so identified can be used beneficially as a markers for genetic screening, mutational analysis and for assessing drug resistance in transformed cells.

### BACKGROUND OF THE INVENTION

Several publications and patent documents are referenced in this application in order to more fully describe the state of the art to which this invention pertains. The disclosure of each of these publications is incorporated by reference herein.

Leukemias including, but not limited to, acute leukemias such as acute lymphoblastic leukemia (ALL) and acute myeloid leukemia (AML) are among the most common malignancies in children. Myelodysplastic syndrome is a designation for a group of syndromes similar to preleukemia (see, e.g. The Merck Manual, 16th ed., Berkow et al., Eds., Merck Research Laboratories, Rahway, N.J., pp. 1243-1245). Leukemias are also a serious cause of morbidity and mortality among adult humans, although MLL gene translocations are present in perhaps only a small proportion of adult acute leukemias. The incidences of ALL and AML in the United States are, respectively, 20 and 10.6 per million individuals per year in infants less than one year old. The aggressiveness with which a leukemia is treated depends, in part, on whether the leukemia has as its genesis a rearrangement of a portion of a chromosome at one or more particular sites. Some translocations may be detected by karyotype analysis, and others cannot be detected by such analysis.

Translocation of the MLL gene (which is alternately designated ALL-1, Htrx1, or HRX) at chromosome band 11q23 is associated with most cases of ALL which occur during infancy and with most monoblastic variants of AML which occur during the first four years of life (Cimino et al., 1993, Blood 82:544-546; Pui et al., 1995, Leukemia 9:762-769; Hilden et al., 1995, Blood 86:3876-3882; Chen et al., 1993, Blood 81:2386-2393; Martinez-Climent et al., 1993, Leukemia 9:1299-1304). About five percent of de novo cases of adult acute leukemia and most DNA topoisomerase II inhibitor-related leukemias are associated with similar translocations (Pui et al., 1995, supra; Martinez-Climent et al., 1993, supra; Raimondi, 1993, Blood 81:2237-2251; Felix et al., 1995, supra).

The MLL gene is 90 kilobases long, comprises 36 exons, and encodes a 3969 amino acid residue protein (Rasio et al., 1996, Cancer Res. 56:1766-1769). The MLL gene is believed to be involved in hematopoiesis and leukemogenesis. The MLL gene product contains several structural motifs important in the regulation of transcription (Domer et al., 1993, Proc. Natl. Acad. Sci. USA 90:7884-7888; Djabali et al., 1992, Nature Genet. 2:113-118; Gu et al., 1992, Cell 71:701-708; Tkachuk et al., 1992, Cell 71:691-700; Ma et al., 1993, Proc. Natl. Acad. Sci. USA 90:6350-6354) and functions as a positive regulator of Hox gene expression (Yu et al., 1995, Nature 378:505-508). Translocation of the MLL gene at chromosome band 11q23 disrupts an 8.3 kilobase breakpoint cluster region (bcr) which is interposed between exons 5 and 11 of MLL. Approximately thirty different translocation partner genes of MLL have been recognized (Martinez-Climent et al., 1993, supra; Raimondi, Blood 81:2237-2251; Felix et al., 1995, Blood 85:3250-3256). Many of these partner genes have not been cloned or characterized.

MLL gene translocations may be detected by karyotype analysis as terminal 11q23 deletions (Shannon et al., 1993, Genes Chromosomes Cancer 7:204-208; Prasad et al., 1993, Cancer Res. 53:5624-5628; Yamamoto et al., 1994, Blood 83:2912-2921). About one third of ALL cases are associated with MLL rearrangements that cannot be detected by karyotype analysis. (Sorenson et al., 1992, Blood 80:255a; Schichman et al., 1994, Proc. Natl. Acad. Sci. USA 91:6236-6239; Schichman et al., 1994, Cancer Res. 54:4277-4280).

Sites of chromosome rearrangement (hereinafter, “breakpoint regions”) have been localized to introns within the bcr of MLL in several de novo cases of leukemia (Gu et al., 1992, Proc. Natl. Acad. Sci. USA 89:10464-10468; Negrini et al., 1993, Cancer Res. 53:4489-4492; Domer et al., 1993, Proc. Natl. Acad. Sci. USA 90:7884-7888; Corral et al., 1993, Proc. Natl. Acad. Sci. USA 90:8538-8542; Gu et al., 1994, Cancer Res. 54:2327-2330). The location of breakpoint regions within MLL and the identity of the nucleotide sequences located at such breakpoint regions are believed to vary according to etiology and pathogenesis of the leukemia. Fewer than half of the about thirty known MLL translocation partner genes have been cloned and identified, although for many of these partner genes, only partial or cDNA sequences are known.

One determinant of the location of a breakpoint region may be the nucleotide sequence preference attributable to either DNA topoisomerase II or a complex comprising DNA topoisomerase II and an agent which interacts with DNA topoisomerase II (Liu et al., 1991, In: DNA Topoisomerases in Cancer, Oxford University Press, New York, pp. 13-22; Ross et al., 1988, In: Important Advances in Oncology, pp.65-79; Pommier et al., 1991, Nucl. Acids Res. 19:5973-5980; Pommier, 1993, Cancer Chemother. Pharmacol. 32:103-108). For example, epipodophyllotoxins form a complex with DNA and DNA topoisomerase II, whereby chromosomal breakage can be effected at the site of complex formation (Corbett et al., 1993, Chem. Res. Toxicol. 6:585-597). Epipodophyllotoxins and other DNA topoisomerase II inhibitors have been associated with leukemias characterized by heterogenous translocations throughout the bcr of MLL at chromosome band 11q23 (Pui et al., 1991, N. Engl. J. Med. 325:1682-1687; Pui et al., 1990 Lancet 336:417-421; Winick et al., J. Clin. Oncol. 11:209-217; Broeker et al., 1996, Blood 87:1912-1922; Felix et al., 1993, Cancer Res. 53:2954-2956; Felix et al., 1995, Blood, 85:3250-3256; Pedersen-Bjergaard, 1992, Leukemia Res. 16:61-65; Pedersen-Bjergaard, 1991, Blood 78:1147-1148).

DNA topoisomerase II catalyzes transient double-strand breakage and religation of genomic DNA, and is involved in regulating DNA topology by relaxation of supercoiled genomic DNA. It is believed that agents which interact with DNA topoisomerase II and which are associated with leukemias inhibit the ability of DNA topoisomerase II to catalyze religation following double-strand breakage. One suggested model for translocations involving MLL entails DNA topoisomerase II-mediated chromosome breakage within the bcr, followed by fusion of DNA free ends from different chromosomes mediated by cellular DNA repair mechanisms (Felix et al., 1995, Cancer Res. 55:4287-4292). Although not strictly inhibitors in the enzymatic sense, epipodophyllotoxins are designated DNA topoisomerase II inhibitors because they decrease the rate of chromosomal religation catalyzed by DNA topoisomerase II and stabilize the DNA topoisomerase II-DNA covalent intermediate (Chen et al., 1994, Annu. Rev. Pharmacol. Toxicol. 84:191-218; Osheroff, 1989, Biochemistry 28:6157-6160; Chen et al., 1984, J. Biol. Chem. 259:13560-13566; Wang et al., 1990, Cell 62:403-406; Long et al., 1985, Cancer Res. 45:3106-3112; Epstein, 1988, Lancet 1:521-524; Osheroff et al., 1991, In: DNA Topoisomerases in Cancer, Potmesil et al., Eds., Oxford University Press, New York, pp. 230-239).

Chromatin structure and scaffold attachment regions may also affect the location of a breakpoint within bcr (Broeker et al., 1996, Blood 87:1912-1922).

Abasic sites are produced by oxidative DNA damage, ionizing radiation, alkylating agents, and spontaneous DNA hydrolysis (Kingma et al., 1995, J. Biol. Chem. 270:21441-21444). Abasic sites are the most common form of spontaneous DNA damage. Abasic sites resulting from exposure to environmental toxins or spontaneous abasic sites may be important mediators of leukemogenesis and provide another explanation of how chromosomal breakage is initiated in leukemia in infants (Kingma et al., 1997, Biochemistry 36:5934-5939), because abasic sites increase DNA topoisomerase II-mediated breakage.

Panhandle PCR methods have been described, and can be used to amplify genomic DNA having a nucleotide sequence comprising a known sequence which flanks an unknown sequence located 3′ with respect to the known sequence (Jones et al., 1993, PCR Meth. Applicat. 2:197-203; U.S. Pat. No. 5,411,875). The panhandle PCR methods comprise generation of a single-stranded DNA having a sequence comprising a region of known sequence at the 5′-end of the single-stranded DNA followed by a region of unknown sequence and having a region complementary to known region DNA at the 3′-end of the single-stranded DNA. The complementary region is complementary to a portion of DNA within the region of known sequence. Thus, the template comprises regions at each end having known sequences. Using primers complementary to each of these regions, the section of the template comprising region of unknown sequence may be amplified, and the nucleotide sequence of this section may be determined. Panhandle PCR has not been used to identify translocation breakpoints or to clone translocation partner genes.

There remains a need for a method of identifying and characterizing chromosomal rearrangements in individual patients afflicted with cancer. Identification and characterization of such a rearrangement in the genome of a patient provides an indication of the type and aggressiveness of the cancer and enables the clinician to better devise suitable therapy strategies to treat the malignancy and symptoms associated therewith. The present invention provides such compositions and methods.

### BRIEF SUMMARY OF THE INVENTION

The invention includes a method of amplifying an unknown region which flanks a known region of a cancer-associated DNA sequence. The method comprises (a) providing a template polynucleotide comprising a sense strand which comprises the known region and the unknown region, wherein the unknown region is nearer the 3′-end of the sense strand than is the known region, wherein the known region comprises a first portion and a second portion, and wherein the first portion is nearer the unknown region than is the second portion; (b) ligating a loop-forming oligonucleotide to the 3′-end of the sense strand, wherein the loop-forming oligonucleotide is complementary to the first portion; (c) annealing the loop-forming oligonucleotide with the first portion to generate a panhandle structure; (d) subjecting the panhandle structure to extension, whereby an additional region complementary to the second portion is generated at the free end of the loop-forming oligonucleotide; and (e) subjecting the panhandle structure to PCR in the presence of a first primer homologous with the second portion, whereby the unknown region is amplified.

In one aspect, the cancer-associated DNA sequence comprises a sequence of a gene shown in Table I.

In another preferred embodiment, the known region comprises a portion of an exon of a breakpoint region selected from exons from the genes listed in Table 1.

In yet another aspect, the first primer has a nucleotide sequence selected from the group consisting of EWS 6f CTCAGCCTGCTTATCCAGCC; EWS 7r GCTATATTGACTTGGAGCTTGGC; EWS 3 GTCAACCTCAATCTAGCACAGGG; FLI3 CTGTCGGAGAGCAGCTCCAG; ERG 3 CTGTCCGACAGGAGCTCAG; FEV 2 GAAACTGCCACAGCTGGATC; ETV 1.1 TAAATTCCATGCCTCGACCAG; E1AF.1 AACTCCATTCCCCGGCC; Pax3.1 TCCAACCCCATGAACCCC; Pax7.1 CAACCACATGAACCCGGTC; FKHR1.2 GCCATTTGGAAAACTGTGATCC; EWS 12 AGCCAACAGAGCAGCAGCTAC; WT1.3 TGAGTCCTGGTGTGGGTCTTC; SYT.2 TACCCAGGGCAGCAAGGTT; SSXc.3 ATCGTTTTGTGGGCCAGATG; ETV6.1 CCCATCAACCTCTCTCATCGG; NTRK3.1 GGCTCCCTCACCCAGTTCTC; ALK.1 AGGTCACTGATGGAGGAGGTCTT; NPM.1 CTTGGGGGCTTTGAAATAACAC; TM30.1 CCGTGCTGAGTTTGCTGAGAG; TFG.1 AGAACCAGGACCTTCCACCAATA; ATIC.1AGGCATTCACTCATACGGCAC; EWS.15 CCCACTAGTTACCCACCCCAAA; TAF68.1 AGCAAAACATGGAATCATCAGGA; TEC.3 TACACGCAGGAAGGCTTGAGTT; ATF1.1 TGTAAGGCTCCATTTGGGGC; EWS S2 CTCCTACCAGCTATTCCTCTACACAGCCGACT; RMS S1 ATGCTCAATCCAGAGGGTGGCAAGAG; WT1 TCTCGTTCAGACCAGCTCAAAAGACACCA; SYNO S1 ATCATGCCCAAGAAGCCAGCAGAGG; FC1 S1 CTCCCCGCCTGAAGAGCACGC; ALK S1 CAAGCTCCGCACCTCGACCATCA; TEC S1 ACCTTGGCAGCACTGAGATCACGGC; BCR (intron 4, forward) GGGCCAAGGAGACCAGTGAGT; BCR (intron 4, reverse) AACAGCCAGCCTGAGGTAGGG; FGFR1 (exons 5-6 forward) ACATCGAGGTGAATGGGAGCAA; FGFR1(exon 12, reverse) TTGGAGGAGAGCTGCTCCTCT; BCR (exon 1, forward) CCCCGGAGTTTTGAGGATTG; and ABL (exon 3) TGGCGTGATGTAGTTGCTTGG.

In yet a further aspect, the panhandle structure is subjected to PCR in the presence of the first primer and further in the presence of a second primer, wherein the second primer is nested with respect to the first primer.

In another aspect, the template polynucleotide further comprises an antisense strand, wherein the 5′-end of the antisense strand overhangs the 3′-end of the sense strand, and wherein a portion of the loop-forming oligonucleotide is complementary to the overhanging region of the antisense strand.

In yet another aspect, the template polynucleotide is provided by obtaining genomic DNA from a patient; contacting the genomic DNA with a restriction endonuclease, whereby a genomic DNA fragment is generated, the genomic DNA fragment comprising the known region and the unknown region, whereby the genomic DNA is the template polynucleotide. The invention also includes a variant method of amplifying an unknown region which flanks a known region of a leukemia-associated DNA sequence. This method comprises (a) providing a template polynucleotide comprising an antisense strand which comprises a region complementary to the known region and a region complementary to the unknown region, wherein the region complementary to the unknown region is nearer the 5′-end of the antisense strand than is the region complementary to the known region, wherein the known region comprises a first portion and a second portion, and wherein the first portion is nearer the unknown region than is the second portion; (b) ligating a first oligonucleotide to the 5′-end of the antisense strand, wherein the first oligonucleotide is homologous with the first portion; (c) annealing a pre-template polynucleotide with the antisense strand, the pre-template polynucleotide being homologous with the second portion; (d) subjecting the pre-template polynucleotide to extension, whereby a sense strand is generated, the sense strand comprising the known region, the unknown region, and a loop-forming oligonucleotide at the 3′-end thereof, the loop-forming oligonucleotide being complementary to the first portion; (e) annealing the loop-forming oligonucleotide with the first portion to generate a panhandle structure; (f) subjecting the panhandle structure to extension, whereby an additional region complementary to the second portion is generated at the free end of the loop-forming oligonucleotide; and (g) subjecting the panhandle structure to PCR in the presence of a first primer homologous with the second portion, whereby the unknown region is amplified.

In one aspect of this aspect of the invention, prior to ligating the first oligonucleotide to the antisense strand, a bridging oligonucleotide is annealed with a portion of the antisense strand adjacent the 5′-end thereof and the first oligonucleotide is annealed with the bridging oligonucleotide.

Also included in the invention is a method of identifying and further characterizing a translocation partner of a cancer-associated DNA sequence, the translocation partner comprising an unknown region, and the cancer-associated DNA sequence comprising a known region. This method comprises (a) providing a template polynucleotide comprising a sense strand which comprises the known region and the unknown region, wherein the unknown region is nearer the 3′-end of the sense strand than is the known region, wherein the known region comprises a first portion and a second portion, and wherein the first portion is nearer the unknown region than is the second portion; (b) ligating a loop-forming oligonucleotide to the 3′-end of the sense strand, wherein the loop-forming oligonucleotide is complementary to the first portion; (c) annealing the loop-forming oligonucleotide with the first portion to generate a panhandle structure; (d) subjecting the panhandle structure to extension, whereby an additional region complementary to the second portion is generated at the free end of the loop-forming oligonucleotide; (e) subjecting the panhandle structure to PCR in the presence of a first primer homologous with the second portion, whereby the unknown region is amplified; and (f) identifying a portion of a human DNA sequence homologous with the unknown region, whereby the human DNA sequence is identified as the translocation partner.

The invention further includes a variant method of identifying a translocation partner of a cancer-associated DNA sequence, the translocation partner comprising an unknown region, and the DNA sequence comprising a known region. This method comprises (a) providing a template polynucleotide comprising an antisense strand which comprises a region complementary to the known region and a region complementary to the unknown region, wherein the region complementary to the unknown region is nearer the 5′-end of the antisense strand than is the region complementary to the known region, wherein the known region comprises a first portion and a second portion, and wherein the first portion is nearer the unknown region than is the second portion; (b) ligating a first oligonucleotide to the 5′-end of the antisense strand, wherein the first oligonucleotide is homologous with the first portion; (c) annealing a pre-template polynucleotide with the antisense strand, the pre-template polynucleotide being homologous with the second portion; (d) subjecting the pre-template polynucleotide to extension, whereby a sense strand is generated, the sense strand comprising the known region, the unknown region, and a loop-forming oligonucleotide at the 3′-end thereof, the loop-forming oligonucleotide being complementary to the first portion; (e) annealing the loop-forming oligonucleotide with the first portion to generate a panhandle structure; (f) subjecting the panhandle structure to extension, whereby an additional region complementary to the second portion is generated at the free end of the loop-forming oligonucleotide; (g) subjecting the panhandle structure to PCR in the presence of a first primer homologous with the second portion, whereby the unknown region is amplified; and (h) identifying a portion of a human DNA sequence homologous with the unknown region, whereby the human DNA sequence is identified as the translocation partner. Also included in the invention is a kit for panhandle PCR amplification of an unknown region of DNA which flanks a known region of the sense strand of a leukemia-associated DNA sequence. The kit comprises an oligonucleotide selected from the group consisting of an oligonucleotide which is complementary to the known region of the sense strand and an oligonucleotide which is homologous with the known region of the sense strand; and a first primer homologous with the known region of the sense strand.

In one aspect, the kit further comprises an internal primer, wherein the internal primer is nested with respect to the first primer, and wherein the internal primer is selected from the group consisting of a primer homologous with the known region of the sense strand.

In another aspect, the kit further comprises at least one recombination PCR primer.

In yet another aspect, the kit further comprises a restriction endonuclease; at least one reagent for ligating the oligonucleotide to a DNA strand obtained from a human patient; at least one reagent for extending a polynucleotide; and at least one reagent for performing PCR.

### BRIEF DESCRIPTION OF THE DRAWINGS

FIG. 1 is a diagram of a ‘basic’ panhandle PCR method described herein.

FIG. 2 is a diagram of a ‘variant’ panhandle PCR method described herein.

FIG. 3 is a diagram showing the structure of the translocation breakpoint region of an infant patient described herein in Example 1.

FIG. 4 is the nucleotide sequence of a translocation breakpoint region described herein in Example 1 (SEQ ID NO: 23).

FIG. 5 is a preliminary nucleotide sequence of a portion of the gene sequence obtained from the unknown region of the amplified polynucleotide product derived from the infant patient described in Example 1 (SEQ ID NO: 1).

FIG. 6 is a preliminary antisense sequence corresponding to the nucleotide sequence in FIG. 5 (SEQ ID NO: 2).

FIG. 7 is a preliminary conglomerate nucleotide sequence of the translocation breakpoint region described herein in Example 1 (SEQ ID NO: 3).

FIG. 8 is a diagram showing the structure of the MLL translocation breakpoint region of the DNA of the patient described in Example 2.

FIG. 9 is a diagram showing the structure of the MLL translocation breakpoint region of the DNA of the patient described in Example 3.

FIG. 10 is a diagram showing the structure of the MLL gene in the DNA of a normal human in the upper portion of the Figure and the structure of the MLL gene in the DNA of the second patient described in Example 3 in the lower portion. Numbers below the structures indicate exon numbers, and the shaded portion represents a portion of the structure of the MLL gene which was duplicated in the DNA of the second patient. BamHI sites are indicated by “B”.

FIG. 11 is a nucleotide sequence of a breakpoint junction of a partial duplication described in Examples 2 and 3 (SEQ ID NO: 15).

FIG. 12 is a nucleotide sequence of a translocation breakpoint junction described in Examples 2 and 3 (SEQ ID NO: 16).

FIG. 13 is a nucleotide sequence of a breakpoint junction of a partial duplication described in Examples 2 and 3 (SEQ ID NO: 17).

FIG. 14 is a nucleotide sequence of a translocation breakpoint junction described in Examples 2 and 3 (SEQ ID NO: 18).

FIG. 15 is a diagram of a translocation breakpoint junction region described herein in Example 7.

FIG. 16 is a schematic diagram depicting reverse panhandle PCR amplification of a genomic breakpoint junction of derivative chromosomes of MLL translocation. In Step 1, genomic DNA is digested with BamHI, producing a restriction fragment with a 5′ overhang, and treated with calf intestinal alkaline phosphatase to prevent religation in Step 2. The BamHI fragment containing the genomic breakpoint junction from the other derivative chromosome has an unknown partner sequence at the 5′ end and MLL sequence at the 3′ end.

In Step 2, a sense phosphorylated oligonucleotide with known MLL sequences from intron 10/exon 11 is ligated to the 3′ ends of the BamHI digested DNA. In Step 3, a stem-loop structure is generated and the antisense strand becomes the template. Heat denaturation renders the DNA single stranded. Intrastrand annealing of the ligated oligonucleotide to the complementary sequence in MLL initiates formation of the handle, which is completed by polymerase extension of the recessed 3′ end. Primer 1 is antisense with respect to MLL exon 11. In Step 4, primer 1 extension during PCR generates the double stranded template from the single-stranded stem-loop structure. MLL sequence and its complement at both ends of the template then enable exponential amplification of the breakpoint junction using primer 1. Steps 5 and 6 enhance yield and specificity through nested, two-sided, single-primer PCR with primers 2 and 3, respectively, which are antisense with respect to exon 11 and exon 11/intron 10 sequences.

FIG. 17 depicts determination of MLL bcr rearrangements in ALL of patient 45 by (A) Southern blot analysis of BamHI-digested DNA with B859 fragment of ALL-1 cDNA (Gu et al., 1992, Cell 71: 701-708) (center) and analysis of panhandle PCR (left panel) and reverse panhandle PCR (right panel) products. The 8.3 kb fragment on the Southern blot depicts the unrearranged MLL allele (center panel, dash); arrows show rearrangements (center panel). (B) Summary of der(11) genomic breakpoint junction sequence in recombination-PCR generated subclones from panhandle PCR. One subclone was sequenced in entirety; the breakpoint junction sequence was verified in three additional subclones. The 5′ 6639 bp include nested MLL forward primer 4 and MLL bcr sequence. 96 bp of 3′ sequence are AF-4 partner DNA. 73 bp of 3′ sequence extend from ligated MLL oligonucleotide (P-Oligo) through nested MLL reverse primer 3 (top). Arrow shows MLL and AF-4 breakpoint positions (bottom). Underlines show short homologies (bottom). Repetitive sequences are shown (middle). (C) Summary of der(4) genomic breakpoint junction sequence in recombination-PCR generated subclone from reverse panhandle PCR. In reverse panhandle PCR, nested primer 3 from MLL intron 10/exon 11 anneals to both ends of the template. 35 bp of 5′ sequence extend from nested MLL primer 3 through ligated oligonucleotide (P-Oligo). 29-30 bp of 5′ sequence are AF-4. The 3′ 2167-2168 bp are MLL bcr sequence through MLL primer 3 (top). Arrowheads show AF-4 and MLL breakpoint positions; ‘A’ nucleotides in both genes precluded precise assignments (bottom). Short homologies are underlined (bottom). Repetitive sequences are shown (middle). One subclone was sequenced in entirety; three PCRs with gene-specific primers confirmed der(4) breakpoint junction.

FIG. 18 depicts determination of MLL bcr rearrangements in ALL of patient t-120 identified by (A) Southern blot analysis of BamHI-digested DNA with B859 cDNA probe (Gu et al., 1992, Cell 71: 701-708) (center) and analysis of panhandle PCR (left panel) and reverse panhandle PCR (right panel) products. The 8.3 kb fragment on the Southern blot (center panel, dash) and the 8.3 kb product in the panhandle PCR (left) are from the unrearranged MLL allele; arrows show rearrangements (center panel). (B) Summary of der(11) genomic breakpoint junction sequence in recombination-PCR generated subclones from panhandle PCR. One subclone was sequenced in entirety; the breakpoint junction sequence was verified in another subclone. The 5′ 6431-6432 bp include nested MLL forward primer 4 and MLL bcr sequence. 790-791 bp of 3′ sequence are AF-4. 73 bp of 3′ sequence extend from ligated MLL oligonucleotide (P-Oligo) through nested MLL reverse primer 3 (top). Arrowheads show AF-4 and MLL breakpoint positions: ‘A’ nucleotides in both genes precluded precise assignments (bottom). Underlines indicate short sequence homologies (bottom). Repetitive sequence elements are shown (middle). (C) Summary of der(4) genomic breakpoint junction sequence in recombination-PCR generated subclones from reverse panhandle PCR. One subclone was sequenced in entirety; the breakpoint junction sequence was verified in another three subclones. 35 bp of 5′ sequence extend from nested MLL primer 3 through ligated oligonucleotide (P-Oligo). 304-306 bp of 5′ sequence are AF-4. The 3′ 1738-1740 bp include MLL bcr sequence through nested MLL primer 3. Arrowheads show AF-4 and MLL breakpoint positions; ‘CA’ nucleotides in both genes precluded precise assignments (bottom). Short sequence homologies are underlined (bottom). Repetitive sequences are shown (middle).

FIG. 19 depicts determination of MLL bcr rearrangements in ALL of patient 38 identified by (A) Southern blot analysis of BamHI-digested DNA with B859 cDNA probe (Felix et al., 1997, Blood 90: 4679-4686; Felix et al., 1998, J Pediatr Hematol/Oncol. 20: 299-308) (arrows, left panel) and analysis of reverse panhandle PCR products (right) consistent with a 2.0 kb rearrangment. The 8.3 kb fragment (dash, left panel) was from the unrearranged MLL allele and the 7.0 kb fragment was from MLL-AF-4 rearrangement (Felix et al., 1997, Blood 90: 4679-4686). (B) Sequence of genomic breakpoint junction of other derivative chromosomes in recombination-PCR generated subclones derived by reverse panhandle PCR. 35 bp of 5′ sequence are from MLL primer 3 through ligated oligonucleotide (P-Oligo). 1028-1030 bp of 5′ sequence are CDK6. The 3′ 1176-1178 bp include MLL bcr sequence from intron 9 through nested MLL primer 3. Arrowheads show CDK6 and MLL breakpoint positions; ‘AG’ nucleotide sequence in both genes precluded precise assignments (bottom). Short sequence homologies are underlined (bottom). Repetitive sequences are shown (middle). (C) Detection of CDK6-MLL fusion transcript. RT-PCR reactions with primers from CDK6 exons 1-2 and MLL exon 13, and randomly primed cDNA template produced a 548 bp product (top). Reactions using β-actin primers and RNA-negative reagent control (dH₂O) are shown (top). Sequencing revealed in-frame fusion of CDK6 exon 2 at position 486 of the 1249 bp CDK6 cDNA (GenBank accession no. NM_{—}001259) to MLL exon 10 (bottom). (D) cdk6 and MLL proteins and predicted cdk6-MLL fusion protein.

FIG. 20 shows representative G-banded karyotype of relapse marrow derived from patient 38. The karyotype was described as 47,XX,t(4;11)(q21;q23),del(7)(q21q31),+8.

FIG. 21(A) shows the MLL bcr rearrangement in AML of patient 62. BamHI-digested DNA was hybridized with B859 fragment of ALL-1 cDNA (Gu et al., 1992). The 8.3 kb fragment indicates an unrearranged MLL allele; arrow shows rearrangement. (B) cDNA panhandle PCR analysis of total RNA from diagnostic marrow of patient 62. Smear in third lane of gel shows products of various sizes from amplification of 5′-MLL-NNNNNN-3′-primed first strand cDNAs with MLL-specific primers (left). The products were subclone by recombination PCR. Thirteen subclones contained an in-frame fusion of MLL exon 7 to SEPTIN6 exon 2. Subclones with SEPTIN6 intron 10 sequences are from incompletely processed transcripts (top right). Subclone with MLL sequence only contains intronic sequence, indicating an incompletely processed transcript (bottom right). (C) Panhandle variant PCR analysis of genomic DNA from diagnostic marrow of patient 62. Three panhandle variant PCRs gave products consistent with MLL bcr rearrangement size on Southern blot (compare with FIG. 21A); gel (left) shows example. Products of one reaction were subcloned by recombination PCR; one subclone was sequenced in entirety. 3103-bp sequence is summarized (right). 31-base sequence of primer 3 from MLL exon 5 used in final round of panhandle variant PCR and complement are at 5′ and 3′ ends. 2514 additional bases of 5′ sequence are from MLL bcr. Corkscrew arrow indicates MLL breakpoint at position 2595 in intron 7. 527 bases of 3′ sequence are from SEPTIN6 intron 1. Alu repeats are shown (middle right). Underlines indicate short homologies (bottom right). (D) Genomic sequence entries in GenBank comprising human SEPTIN6. Each GenBank entry appears in reverse complement (rc) from orientation of transcription.

FIG. 22 shows SKY analysis of exemplary metaphase cell from marrow of patient 62 at AML diagnosis. The chromosomes are arranged in karyotype fashion. Inverted DAPI-image (left) and the respective SKY-classification (right) are shown for each chromosome. SKY analysis of ten metaphases was interpreted as 47,X,der(X)t(X;11)(q22;q23)t(3;11)(p21;q12),der(3) t(3;11)(p21;q23)t(X;11)(q22;q25),+6,der(11)del(11)(q12?qter).

FIG. 23 depicts metaphase FISH analysis of AML of patient 62. FISH analysis of a metaphase cell with painting probes for chromosome 3 (green), chromosome 11 (red), a centromere probe for the X chromosome (blue), and a probe for MLL (yellow) (left) confirmed the complex translocation detected by SKY. Simultaneous hybridization with a probe for MLL (yellow) showed MLL signals on the normal chromosome 11, the der(X) and the der(3). The image at right shows the der(3) chromosome from a metaphase chromosome with enhanced resolution. The signals for MLL (yellow) are located at the interface between material from chromosome 3 (green) and chromosome 11 (red). These results indicate that the 5′-MLL-SEPTIN6-3′ junction identified by panhandle variant PCR was on the der(X).

FIG. 24(A) shows an autoradiograph of cleavage products generated during a DNA topoisomerase II cleavage assay of MLL intron 7/exon 8 coordinates 2490 to 3077 containing the normal homologue of the MLL genomic breakpoint in AML of patient 62. Cleavage products were isolated after a 10 minute incubation of 25 ng (30,000 cpm) singly 5′ end-labeled DNA with 147 nM human DNA topoisomerase IIα, 1 mM ATP and, where indicated, 20 μM etoposide (VP16). Heat indicates reactions incubated for 10 minutes at 65° C. before trapping of covalent complexes. The indicated nucleotide (MLL position 2595), which was the translocation breakpoint, was the 5′ side or −1 position of a cleavage site (bold arrow); the cleaved phosphodiester bond is 3′ to this position. The DNA topoisomerase II inhibitor etoposide enhanced cleavage at this site 1.2-fold. Detection of cleavage after heating to 65° C. indicates stability of the cleavage complex formed at this position. (B) Summary of DNA topoisomerase II in vitro cleavage sites proximal to MLL genomic breakpoint in AML of patient 62. Dots indicate bases at 5′ side (−1 position) of cleavage sites identified. Numbers are relative nucleotide positions in normal genomic sequence. Arrow indicates correspondence of normal homologue of the translocation breakpoint to DNA topoisomerase II cleavage site.

FIG. 25(A) shows a Southern blot depicting the MLL bcr rearrangement in AML of patient 23. BamHI-digested DNA from marrow at AML diagnosis was hybridized with B859 fragment of ALL-1 cDNA (Gu et al., 1992). The 8.3 kb fragment indicates an unrearranged MLL allele; arrows show two rearrangements. (B) cDNA panhandle PCR analysis of total RNA from diagnostic marrow of patient 23. Smear in third lane of gel shows products of various sizes from amplification of 5′-MLL-NNNNNN-3′-primed first strand cDNAs with MLL-specific primers (left). The products were subcloned by recombination PCR. Nine subclones contained an in-frame fusion of MLL exon 8 to SEPTIN6 exon 2. Subclones with SEPTIN6 intron 3 in sequence are from incompletely processed transcripts (top right). Other subclones contained only MLL (bottom right).

### DETAILED DESCRIPTION OF THE INVENTION

The invention relates to kits and methods for panhandle PCR amplification of a region of DNA having an unknown nucleotide sequence, wherein the region flanks a region of a cancer-associated gene having a known nucleotide sequence in the DNA of a human patient. Two different panhandle PCR methods have been discovered. Amplification of an unknown region flanking a known region of a cancer-associated gene permits identification of a translocation partner of the gene or identification of a duplicated sequence within the gene. Identification of the translocation partner or the duplicated sequence permits a medical practitioner to predict the course of a cancer associated with the presence of the translocation partner or the duplicated sequence, and further permits the practitioner to determine the aggressiveness of anti-cancer therapy that will be required. The invention further relates to kits useful for performing the methods of the invention.

### Definitions

As used herein, the following terms have the meanings described in the present application.

The “bcr” region of MLL means the breakpoint cluster region of the MLL gene, an approximately 8.3-kilobase region of the gene which extends from a BamHI cleavage site of the sense strand of MLL exon 5 to another BamHI cleavage site of the sense strand of MLL exon 11. The sequence of the bcr of MLL is known (GenBank Accession # HSU04737). Where nucleotide residues are numbered within the bcr, they are numbered from the 5′-end of the sense strand of the bcr of MLL. Where breakpoints are identified within the bcr of MLL, the location of the breakpoint refers to the nucleotide residue located immediately 5′ of the site of breakage (i.e. the 3′-most residue of wild type MLL sequence following the translocation event).

A first region of a polynucleotide “flanks” a second region of the polynucleotide if the two regions are adjacent to one another, or if the two regions are separated by no more than about 1000 nucleotide residues, and preferably by no more than about 100 nucleotide residues.

A first region of a polynucleotide is “adjacent” to a second region of the polynucleotide if the two regions are attached to or positioned next to one another, having no intervening nucleotides. By way of example, the pentanucleotide region 5′-AAAAA-3′is adjacent to the trinucleotide region 5′-TTT-3′ when the two are connected thus: 5′-AAAAATTT-3′ or 5′-TTTAAAAA-3′, but not when the two are connected thus: 5′-AAAAACTTT-3′.

“Complementary” refers to the broad concept of subunit sequence complementarity between regions of two polynucleotides or between two regions of the same polynucleotide. It is known that an adenine residue of a first polynucleotide region is capable of forming specific hydrogen bonds (“base pairing”) with a residue of a second polynucleotide region which is antiparallel to the first region if the residue is thymine or uracil. Similarly, it is known that a cytosine residue of a first polynucleotide region is capable of base pairing with a residue of a second polynucleotide region which is antiparallel to the first region if the residue is guanine. A first region of a polynucleotide is complementary to a second region of the same or a different polynucleotide if, when the two regions are arranged in an antiparallel fashion, at least three nucleotide residues of the first region is capable of base pairing with three residues of the second region. Preferably, the first region comprises a first portion and the second region comprises a second portion, whereby, when the first and second portions are arranged in an antiparallel fashion, at least about 30%, and preferably at least about 75%, at least about 90%, or at least about 95% of the nucleotide residues of the first portion are capable of base pairing with nucleotide residues in the second portion. Such portions are said to exhibit 30%, 75%, 90%, and 95% complementarity, respectively. More preferably, all nucleotide residues of the first portion are capable of base pairing with nucleotide residues in the second portion (i.e. the first and second portions exhibit 100% complementarity).

A first polynucleotide region and a second polynucleotide region are “arranged in an antiparallel fashion” if, when the first region is fixed in space and extends in a direction from its 5′-end to its 3′-end, at least a portion of the second region lies parallel to the first region and extends in the same direction from its 3′-end to its 5′-end.

“Homologous” as used herein, refers to nucleotide sequence identity between two regions of the same polynucleotide or between regions of two different polynucleotides. When a nucleotide residue position in both regions is occupied by the same nucleotide residue, then the regions are homologous at that position. A first region is homologous to a second region if at least three nucleotide residue positions of each region are occupied by identical nucleotide residues. Homology between two regions is expressed in terms of the proportion of nucleotide residue positions of the two regions that are occupied by the same nucleotide residue. By way of example, a region having the nucleotide sequence 5′-ATTGCC-3′ and a region having the nucleotide sequence 5′-TATGGC-3′ are 50% homologous. Preferably, the first region comprises a first portion and the second region comprises a second portion, whereby, at least about 50%, and preferably at least about 75%, at least about 90%, or at least about 95% of the nucleotide residue positions of each of the portions are occupied by the same nucleotide residue. Such portions are said to exhibit 50%, 75%, 90%, and 95% homology, respectively More preferably, all nucleotide residue positions of each of the portions are occupied by the same nucleotide residue (i.e. the first and second portions exhibit 100% homologous).

A “cancer-associated DNA sequence” means a DNA sequence of a human patient wherein translocation of genomic DNA into the DNA sequence or rearrangement of the DNA sequence is associated with onset, continuation, or relapse of cancer in the patient. In certain embodiments, leukemia-associated DNA sequences are described which include, but are not limited to, genes, such as MLL, which are associated with onset, continuation, or relapse of acute leukemia. It is understood that changes in a leukemia-associated DNA sequence, such as a chromosomal translocation for example, may occur in a preleukemia phase before leukemia is clinically detected. It is also understood that the identification of chromosomal rearrangements associated with additional types of cancers aids the clinician in the design of appropriate therapeutic intervention modalities.

A “region” and a “portion” of a polynucleotide are used interchangeably to mean a plurality of sequential nucleotide residues of the polynucleotide.

A “polynucleotide” means a single strand or parallel and anti-parallel strands of a nucleic acid. Thus, a polynucleotide may be either a single-stranded or a double-stranded nucleic acid.

An “oligonucleotide” means a nucleic acid comprising at least two nucleotide residues.

A first polynucleotide is “ligated” to a second polynucleotide if an end of the first polynucleotide is covalently bonded to an end of the second polynucleotide. By way of example, the covalent bond may be a phosphodiester bond.

“Extending” a polynucleotide means the addition of nucleotide residues to an end of the polynucleotide, wherein the added nucleotide residues are complementary to nucleotide residues of a region of either the same or a different polynucleotide with which the polynucleotide is annealed. Extension of a polynucleotide typically occurs by template-directed polymerization or by template-directed ligation.

A first polynucleotide is “annealed” with a second polynucleotide when the two polynucleotides are arranged in an anti-parallel fashion and when at least three nucleotide residues of the first polynucleotide are base paired with a nucleotide residue of the second polynucleotide.

A “panhandle structure” is a polynucleotide comprising a first region and a second region, wherein when the first region and the second region are separated by at least several nucleotide residues and are annealed to each other in an anti-parallel fashion. The first and second regions may be separated by several hundred or even by several thousand nucleotide residues.

A “primer” is an oligonucleotide which can be extended when annealed with a complementary region of a nucleic acid strand.

“Amplification of a region of a polynucleotide” means production of a plurality of nucleic acid strands comprising the region.

A “product” of an amplification reaction such as PCR means an polynucleotide generated by extension of a primer used in the amplification reaction.

A first polynucleotide comprises an “overhanging region” if it has a double-stranded portion wherein either the 3′-end or the 5′-end of a strand of the polynucleotide extends beyond the 5′-end or the 3′-end, respectively, of the same or a different strand of the polynucleotide. By way of example, the 5′-end of an antisense strand overhangs the 3′-end of a sense strand with which it is annealed if the 5′-end of the antisense strand extends beyond the 3′-end of the sense strand.

A “genomic DNA” of a human patient is a DNA strand which has a nucleotide sequence homologous with or complementary to a portion of a chromosome of the patient. Included in this definition for the purposes of simplicity are both a fragment of a chromosome and a cDNA derived by reverse transcription of a human RNA.

A “translocation partner” of a human gene is a region of genomic DNA which does not normally flank the gene, but which flanks the gene following a translocation event. A “translocation event” means fusion of a first region of a human chromosome with a second region of a human chromosome, wherein the first region and the second region are not normally fused. By way of example, breakage of a first and a second human chromosome and fusion of a part of the first chromosome with a part of the second chromosome is a translocation event. For the sake of simplicity, tandem duplications are herein included within the definition of translocation event, it being understood that tandem duplications and translocations occur by similar mechanisms of DNA recombination.

A polynucleotide is “derived from” a gene if the polynucleotide has a nucleotide sequence which is either homologous with or complementary to a portion of the nucleotide sequence of the gene.

A first polynucleotide anneals with a second polynucleotide “with high stringency” if the two oligonucleotides anneal under conditions whereby only oligonucleotides which are at least about 75%, and preferably at least about 90% or at least about 95%, complementary anneal with one another. The stringency of conditions used to anneal two oligonucleotides is a function of, among other factors, temperature, ionic strength of the annealing medium, the incubation period, the length of the oligonucleotides, the G-C content of the oligonucleotides, and the expected degree of non-homology between the two oligonucleotides, if known. Methods of adjusting the stringency of annealing conditions are known (see, e.g. Sambrook et al., 1989, Molecular Cloning: A Laboratory Manual, Cold Spring Harbor Laboratory, New York).

A third primer is “nested” with respect to a first primer and a second primer if amplification of a region of a first polynucleotide using the first primer and the second primer yields a second polynucleotide, wherein the third primer is complementary to an internal portion of the second polynucleotide, wherein the internal portion of the second polynucleotide to which it is complementary does not include a nucleotide residue at the corresponding end of the second polynucleotide.

A second primer is “nested” with respect to a first primer if amplification of a region of a first polynucleotide using the first primer yields a second polynucleotide, wherein the second primer is complementary to an internal portion of the second polynucleotide, and wherein the internal portion of the second polynucleotide to which the second primer is complementary does not include a nucleotide residue at the corresponding end of the second polynucleotide.

A portion of a polynucleotide is “near” the end of a region of the polynucleotide if at least one nucleotide residue of the portion is separated from the end of the region by no more than about one hundred nucleotide residues, and preferably by no more than about twenty-five nucleotide residues.

A restriction site is a portion of a polynucleotide which is recognized by a restriction endonuclease.

A portion of a polynucleotide is “recognized” by a restriction endonuclease if the endonuclease is capable of cleaving a strand of the polynucleotide at a fixed position with respect to the portion of the polynucleotide.

A strand of a polynucleotide is the “sense” strand with respect to unknown flanking DNA if the nucleotide sequence of a first portion of the strand is known, if the nucleotide sequence of a second portion of the strand is unknown, and if the first portion is located 5′ with respect to the second portion.

### Description

The invention includes a ‘basic’ panhandle PCR method and a ‘variant’ panhandle PCR method. Either of the basic panhandle PCR method of the invention or the variant panhandle PCR method of the invention can be used to amplify an unknown region which flanks a known region of a cancer-associated gene or to identify a translocation partner of such a gene.

Basic Panhandle PCR Method

The basic panhandle PCR method of the invention can be used to amplify an unknown region which flanks a known region of a cancer-associated DNA sequence as follows.

A template polynucleotide is provided, the template polynucleotide comprising a sense strand which comprises the known region of a cancer-associated DNA sequence and an unknown region which flanks the DNA sequence. The unknown region is nearer the 3′-end of the sense strand of the template polynucleotide than is the known region of the DNA sequence. Two portions of the known region of the DNA sequence are designated a first portion and a second portion, the first portion being nearer the unknown region than is the second portion. The template polynucleotide preferably comprises a known region of at least about twenty nucleotides. In embodiments where the cancer is leukemia, the cancer-associated DNA sequence is preferably MLL. The known region may be, for example, a portion of the breakpoint cluster region of MLL, a portion of MLL which flanks the breakpoint cluster region of MLL, a portion of an exon of MLL such as a portion of exon 5 or a portion of exon 11, or a portion of an intron of MLL.

The template polynucleotide may be provided in the form of single-stranded or double-stranded DNA. When the template polynucleotide is double-stranded DNA, the 5′-end of the antisense strand may overhang the 3′-end of the sense strand. The method used to obtain the template polynucleotide is not critical. Many methods are known for generating or isolating DNA suitable for use as template DNA. By way of example, the template polynucleotide may be provided by obtaining genomic DNA from a patient, contacting the genomic DNA with a restriction endonuclease, whereby a genomic DNA fragment is generated, the genomic DNA fragment comprising the known region. This fragment may be used as the template polynucleotide.

A loop-forming oligonucleotide is ligated to the 3′-end of the sense strand of the template polynucleotide, the loop-forming oligonucleotide being complementary to the first portion of the known region of the DNA sequence. After ligating the loop-forming oligonucleotide to the template polynucleotide, the unknown region is flanked on one side by the loop-forming oligonucleotide and on the other side by the known region of the gene. The loop-forming oligonucleotide is then annealed with the first portion of the known region to generate a panhandle structure. When the template polynucleotide is provided in the form of double-stranded DNA, ligation of the loop-forming oligonucleotide to the sense strand of the template polynucleotide may be more easily achieved if the overhanging portion of the antisense strand of the template polynucleotide is complementary to one or more nucleotide residues at the 5′-end of the loop-forming oligonucleotide. Furthermore, it is necessary to denature a double-stranded template polynucleotide prior to annealing the loop-forming oligonucleotide with the first portion of the known region.

In the panhandle structure, the known region of the cancer-associated DNA sequence and the loop-forming oligonucleotide form a “handle” region” of duplex DNA, and the unknown region is located in a single-strand “pan” region of the structure which is bounded on each end by one of the two DNA strands of the “handle” region. If the panhandle structure is subjected to extension, then a third region, complementary to the second portion is attached to the free end of the loop-forming oligonucleotide, such that the double-stranded “handle” portion of the panhandle structure further comprises the second portion of the known region of the DNA sequence and its complement. Thus, a DNA strand having known nucleotide sequences at each end and the unknown region therebetween is generated.

The DNA strand thus generated may be amplified by conventional PCR techniques, using one or more primers, such that at least one primer is homologous with a portion of the known region. The conventional PCR techniques may be, for example, long distance PCR techniques. The loop-forming oligonucleotide and the third region are complementary to the first portion and the second portion, respectively, of the known region of the sense strand. A single primer which is homologous with the first or the second portion of the known region can be annealed with a first DNA strand generated by extension of the panhandle structure or a second DNA strand generated by amplification of the first strand and the strand complementary to the first strand. Primers which can be used to amplify the DNA strand include, but are not limited to, a primer homologous with the second portion of the known region, a primer homologous with the first portion of the known region, and a primer homologous with a known portion of the “pan” region of the sense strand. Amplification of the DNA strand results in amplification of the unknown region.

Variant Panhandle PCR Method

The variant panhandle PCR method of the invention is an embodiment of panhandle PCR, and can, like the basic panhandle PCR method of the invention, be used to amplify an unknown region which flanks a known region of a cancer-associated DNA sequence as follows.

A template polynucleotide is provided, the template polynucleotide comprising an antisense strand which comprises a region complementary to the known region of a cancer-associated DNA sequence and a region complementary to an unknown region which flanks the DNA sequence. The region complementary to the unknown region is nearer the 5′-end of the antisense strand of the template polynucleotide than is the region complementary to the known region of the DNA sequence. Two portions of the known region of the DNA sequence are designated a first portion and a second portion, the first portion being nearer the unknown region than is the second portion. The template polynucleotide preferably comprises at least about twenty nucleotides complementary to the known region of the sense strand. The cancer-associated DNA sequence is preferably MLL. The known region may be, for example, a portion of the breakpoint cluster region of MLL, a portion of MLL which flanks the breakpoint cluster region of MLL, a portion of an exon of MLL such as a portion of exon 5 or a portion of exon 11, or a portion of an intron of MLL.

The template polynucleotide may be provided in the form of single-stranded or double-stranded DNA. When the template polynucleotide is double-stranded DNA, the 5′-end of the antisense strand may overhang the 3′-end of the sense strand or the 3′-end of the sense strand may overhang the 5′-end of the antisense strand. The method used to obtain the template polynucleotide is not critical. Many methods are known for generating or isolating DNA suitable for use as template DNA. By way of example, the template polynucleotide may be provided by obtaining genomic DNA from a patient, contacting the genomic DNA with a restriction endonuclease, whereby a genomic DNA fragment is generated, the genomic DNA fragment comprising the known region. This fragment may be used as the template polynucleotide.

A first oligonucleotide is ligated to the 5′-end of the antisense strand of the template polynucleotide, the first oligonucleotide being homologous with the first portion of the known region of the sense strand of the DNA sequence. After ligating the first oligonucleotide to the template polynucleotide, the unknown region of the antisense strand is flanked on one side by the first oligonucleotide and on the other side by a polynucleotide complementary to the known region of the gene. A pre-template polynucleotide is annealed with the antisense strand, the pre-template polynucleotide being homologous with at least part of the second portion of the known region of the DNA sequence. The pre-template polynucleotide may, for example, be a primer homologous with part of the second portion, or a sense strand of the template polynucleotide. The pre-template polynucleotide is subjected to extension, whereby a sense strand is generated, the sense strand comprising the known region, the unknown region, and a loop-forming oligonucleotide at the 3′-end thereof. The loop-forming oligonucleotide is the complement of the first oligonucleotide and is complementary to the first portion of the known region.

The loop-forming oligonucleotide is then annealed with the first portion of the known region of the sense strand to cause the sense strand to assume a panhandle structure. Ligation of the first oligonucleotide to the antisense strand may be easier if a bridging oligonucleotide is used, wherein the bridging oligonucleotide is complementary to a portion of the antisense strand at the 5′-end thereof, and wherein the bridging oligonucleotide is complementary to the first oligonucleotide. By annealing the antisense strand, the bridging oligonucleotide, and the first oligonucleotide, the 3′-end of the first oligonucleotide may be positioned adjacent the 5′-end of the antisense strand.

In the panhandle structure, the known region of the cancer-associated DNA sequence and the loop-forming oligonucleotide form a “handle” region” of duplex DNA, and the unknown region is located in a single-strand “pan” region of the structure which is bounded on each end by one of the two DNA strands of the “handle” region. If the panhandle structure is subjected to extension, then a third region, complementary to the second portion is attached to the free end of the loop-forming oligonucleotide, such that the double-stranded “handle” portion of the panhandle structure further comprises the second portion of the known region of the DNA sequence and its complement. Thus, a DNA strand having known nucleotide sequences at each end and the unknown region therebetween is generated.

The DNA strand thus generated may be amplified by conventional PCR techniques, using one or more primers, such that at least one primer is homologous with a portion of the known region. The conventional PCR technique may, for example, be a long-distance PCR technique. The loop-forming oligonucleotide and the third region are complementary to the first portion and the second portion, respectively, of the known region. A single primer which is homologous with the second or the first portion of the known region can be annealed with a first DNA strand generated by extension of the panhandle structure or a second DNA strand generated by amplification of the first strand and the strand complementary to the first strand. Primers which can be used to amplify the DNA strand include, but are not limited to, a primer homologous with the first portion of the known region, a primer homologous with the second portion of the known region, and a primer homologous with a known portion of the “pan” region of the sense strand. Amplification of the DNA strand results in amplification of the unknown region.

Alternate Embodiments of the Panhandle PCR Methods of the Invention

Certain embodiments of the panhandle PCR methods of the invention are now described. It is understood that the methods of the invention are not limited to the particular embodiments illustrated herein, but should be construed to include equivalent methods and variations thereof which can be designed by those skilled in the art upon a reading of the present disclosure.

Cloning of MLL genomic breakpoint regions by PCR methods has been difficult because, although each breakpoint region on the derivative 11 (“der(11)”) chromosome comprises a known 5′ sequence from MLL, PCR primers could not be designed which were consistently specific for all of the many 3′ breakpoint region sequences derived from unknown partner DNA sequences, including sequences derived from coding regions of genes, sequences derived from non-coding regions of genes, and sequences derived from intergenic DNA sequences. It has been estimated that no fewer than thirty different partner genes are involved in MLL translocation (Pui et al., 1995, Leukemia 9:762-769). Although fourteen partner genes of MLL have been cloned, including those described herein, partner gene sequence information is, in many cases, limited to cDNA sequences (Bernard et al., 1994, Oncogene 9:1039-1045; Nakamura et al., 1993, Proc. Natl. Acad. Sci. USA 90:4631-4635; Rubnitz et al., 1994, Blood 84:1747-1752; Prasad et al., 1993, Cancer Res. 53:5624-5628; Thirman et al., 1994, Proc. Natl. Acad. Sci. USA 91:12110-12114; Tse et al., 1995, Blood 85:650-656; Chaplin et al., 1995, Blood 86:2073-2076; Chaplin et al., 1995, Blood 85:1435-1441; Parry et al., 1994, Genes Chromosom. Cancer 11:79-84; Taki et al., 1997, Blood 89:3945-3950; Sobulo et al., 1997, Proc. Natl. Acad. Sci. USA 94:8732-8737; So et al., 1997, Proc. Natl. Acad. Sci. USA 99:2563-2568; Hillion et al., 1997, Blood 9:3714-3719; Borkhardt et al., 1997, Oncogene 14:195-202). The nucleotide sequences of the remaining partner genes have not yet been determined and, thus, are not available for design of primers for genomic breakpoint region cloning.

In approximately one-third of patients who exhibit molecular MLL gene rearrangement by Southern blot analysis, karyotype analysis cannot detect the translocation or provide information about potential translocation partners. Other translocation events involve partial tandem duplication of one or more regions of MLL (Schichman et al., 1994, Proc. Natl. Acad. Sci. USA 91:6236-6239; Schichman et al., 1994, Cancer Res. 54:4277-4280), and are not detectable on karyotype analysis.

For these reasons, a set of conventional PCR primers cannot reasonably be designed such that the primers can be used to amplify all possible MLL genomic breakpoint regions. The panhandle PCR methods of the invention overcome the limitations of conventional PCR methods for amplification of cancer-associated gene breakpoint regions. The methods described herein have been used to clone breakpoint regions comprising the MLL bcr in numerous patients. Nonetheless, it is clear that the methods of the invention can be used analogously to clone breakpoint region(s) of any cancer-associated gene and other genes involved in translocations, whether somatic or constitutional in nature, and whether involved in cancer or other disease states (e.g. Look et al., 1997, In: Principles and Practices of Pediatric Oncology, 3rd ed., Pizzo et al., Eds, Lippincott-Raven Publishers, Philadelphia, Pa., Chapter 3). In MLL, the bcr is located in an 8.3 kilobase region interposed between exons 5 and 11 and bounded by BamHI sites at either end. The length of the bcr of MLL is suitable for amplification by the panhandle PCR methods of the invention.

Gale et al. demonstrated that MLL gene rearrangements involving MLL and AF4 may be detectable at birth by conventional PCR, several months or even years before the onset of leukemia (Gale et al., 1997, Proc. Natl. Acad. Sci. USA 94:13950-13954). However, MLL has many translocation partners, and no PCR primer set could amplify all possible translocations. Furthermore other methods such as Southern blot analysis, fluorescent in situ hybridization, and cytogenetic methods are not as sensitive as PCR methods for detecting translocation events.

Latency to onset of clinical disease in both infants and in patients with treatment-related leukemias with MLL gene translocations provides an opportunity for leukemia prevention by pre-leukemia screening and detection before cells with the translocation establish clonal dominance. The panhandle PCR methods of the invention have the sensitivity necessary for diagnostic use to detect MLL gene rearrangements before the onset of leukemia. The diagnostic capability of the panhandle PCR methods of the invention represents a significant advance relative to prior art gene rearrangement detection methods.

A single diagnostic test using a panhandle PCR method can screen for a panoply of translocation events. Isolation of one of numerous breakpoints and partner sequences can yield sequence information that is informative with regard to both diagnosis and prognosis. For example, for MLL, with its many translocation partners, such a diagnostic test does not exist.

Preliminary experiments involving serial dilutions indicate that the panhandle PCR methods of the invention can be used to detect an MLL gene translocation in an amount of DNA equivalent to the amount of DNA in as few as about thirty cells. These results were obtained without optimization of the detection system used. Thus, it is believed that the panhandle PCR methods of the invention may be useful for detecting translocation events in fewer than thirty cells. Furthermore, the usefulness of the methods of identifying the partner gene involved in a cancer-associated translocation event may become even more apparent as the methods described herein and other methods are used to gather data regarding clinical outcomes associated with the identities of various partner genes. As this information base develops, the methods of the invention can be used to predict clinical outcomes in individual patients, and to assist practitioners to select an appropriate course of treatment. The exemplary panhandle PCR methods of the invention have the advantage of amplifying all MLL gene rearrangements without the need for primers for the many partner genes of MLL, and thus for pre-clinical detection and characterization of leukemia once disease is evident, and subsequent monitoring of the disease.

The panhandle PCR methods of the invention have been devised to simplify PCR-based cloning of genomic DNA having unknown sequences flanking known sequences, which is the case with many cancer-assocated genomic breakpoint regions.

One Embodiment of the Panhandle PCR Methods of the Invention

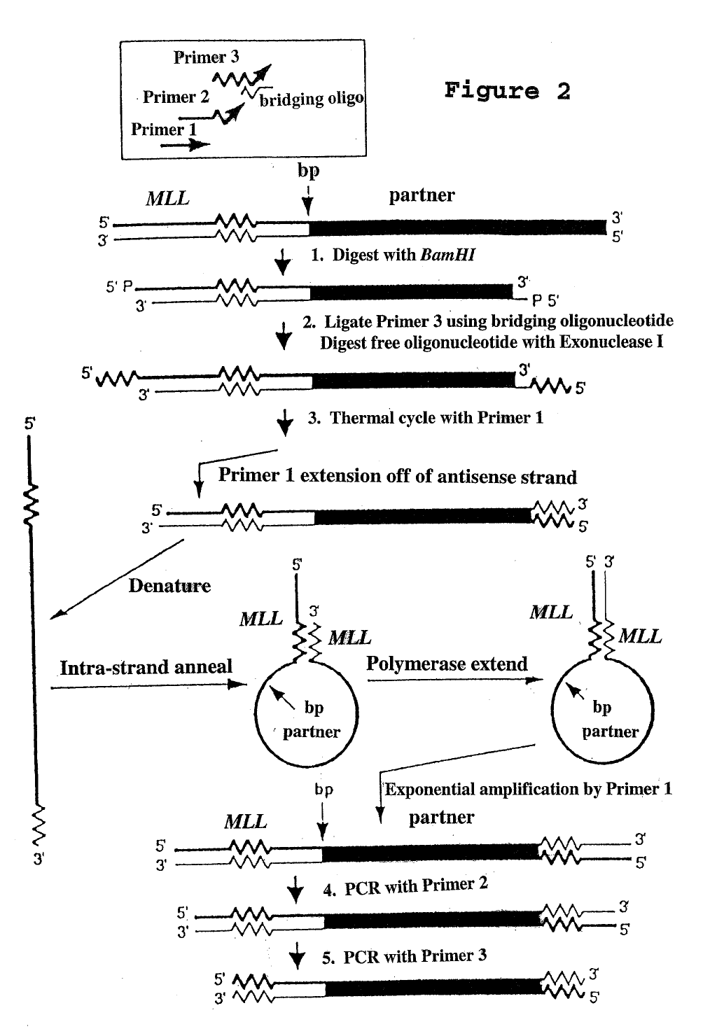

In one embodiment represented in FIG. 2, the variant panhandle PCR method of the invention is performed as follows.

Genomic DNA is obtained from a patient afflicted with leukemia and is digested to completion using the restriction endonuclease BamHI. This treatment generates a plurality of genomic DNA fragments, each having an overhanging region, whereby the 5′-end of each strand overhangs the 3′-end of the strand with which it is annealed.

A single-stranded first oligonucleotide that is homologous to a known sense MLL genomic sequence (designated “Primer 3” in this embodiment) is ligated to the 5′ ends of the BamHI-digested genomic DNA fragments. A bridging oligonucleotide which is complementary to the four-nucleotide-residue overhanging region at one end and complementary to the first oligonucleotide at its other end facilitates the ligation. Primer 3 may be, for example, a 31-nucleotide first oligonucleotide homologous to nucleotides 51 through 81 of the bcr of MLL, in MLL exon 5. The purpose of the bridging oligonucleotide is to position the 3′-end of the first oligonucleotide adjacent each 5′-end of the BamHI-digested genomic DNA fragment. BamHI-digested genomic DNA fragments are added directly to the ligation reaction mixtures, i.e., without purifying fragments from the digestion reaction mixture. After ligation is completed, the bridging oligonucleotide and non-ligated first oligonucleotide may be degraded by addition of exonuclease I to the ligation mixture, which results in digestion of these oligonucleotides.

As represented in step 3 in FIG. 2, a primer (designated “Primer 1” in this embodiment) is used to generate a sense strand by extension of Primer 1 in the presence of the antisense strand of the template polynucleotide. Primer 1 is homologous with a portion of MLL, such as for example, the portion of MLL consisting of nucleotide residues 34 to 55 of the bcr of MLL, in MLL exon 5. Thus, a sense strand (the upper strand in FIG. 2 following step 3) is generated, comprising the known region of the cancer-associated DNA sequence, the unknown region, and a loop-forming oligonucleotide at the 3′-end thereof. The loop-forming oligonucleotide is complementary to the first portion of the known region.

Heat denaturation can be used to dissociate the antisense strand of the template polynucleotide from the sense strand. Thereafter, intrastrand annealing of the loop-forming oligonucleotide with the first portion generates a panhandle structure. Extension of the recessed 3′-end of the panhandle structure completes generation of the panhandle structure. The intrastrand “pan” portion of the panhandle structure comprises the breakpoint region and the unknown partner DNA, while the handle comprises a known region of the template polynucleotide homologous with the sense strand of MLL and a region complementary to the sense strand of MLL.

PCR amplification of the panhandle structure in the presence of Primer 1, which anneals both at the 3′-end of the sense strand and at the 3′-end of the antisense strand of the template polynucleotide, exponentially amplifies the template polynucleotide, including the breakpoint region and the unknown partner DNA.

As represented by steps 4 and 5 in FIG. 2, further PCR amplification using one or more primers, each of which is nested with respect to Primer 1, can be performed to increase the yield and the specificity of the method. For example, two sequential nested, single-primer PCR amplifications may be performed, the first amplification being performed in the presence of an internal primer designated “Primer 2” in this embodiment, and the second amplification being performed in the presence of Primer 3. Primer 2 may, for example, be homologous with positions 38-61 of the bcr of MLL, in MLL exon 5. Primer 3 may, for example, be homologous with nucleotide residues 51 to 81 of the bcr of MLL.

Subcloning of the amplified polynucleotide product generated by an embodiment of either the basic or the variant panhandle PCR method of the invention may be desirable if the yield of the amplified polynucleotide product is not considered sufficient. When subcloning is desired, a simple and efficient method, herein designated “recombination PCR” may be used. Recombination PCR relates to the fact that E. coli mediates DNA recombination and that DNA ends comprising short regions of homology can undergo intra- and intermolecular recombination in vivo in E. coli, including, but not limited to, in RecA-deficient strains such as those routinely used for Subcloning (Jones et al., 1991, BioTechniques 10:62-66). To perform subcloning by recombination PCR, PCR is performed using a HindIII-digested pUC19 plasmid template and primers having 5′-ends complementary to the primer used to generate the basic or the variant panhandle PCR product (Jones et al., 1991, BioTechniques 10:62-66). PCR products from both the panhandle PCR reaction and the pUC19 amplification are combined, they undergo in vivo recombination when transformed into E. coli with the desired recombinant plasmid. To identify recombinant plasmids containing products of basic or variant panhandle PCR, genomic subclones are screened by PCR rather than by preparing and digesting miniprep DNAs, as in conventional methods. Preliminary results indicate that this approach is faster than conventional subcloning methods.

It is anticipated that the panhandle PCR methods of the invention will lead to discovery of new MLL translocation partner genes. The panhandle PCR methods of the invention can be used to identify partner genes in leukemias associated with cytogenetic translocations involving bands 10q11 or Xq22 where no partner genes have yet been cloned.

Panhandle PCR methods have been used to amplify polynucleotides from about 2 to about 4.4 kilobases in length (Jones et al., 1992, Nucl. Acids Res. 20:595-600; Jones, 1995, PCR Meth. Applicat. 4:S195-S201; Jones, et al., 1993, PCR Meth. Applicat. 2:197-203). The variant panhandle PCR method of the invention has been used to amplify polynucleotides comprising MLL gene translocations from about 3.9 to about 8.3 kilobases in length. The basic panhandle PCR method of the invention has been used to amplify products comprising MLL gene translocations from about 2.5 to about 8.3 kb in length. The maximum length of the polynucleotide that can be amplified using the panhandle PCR methods of the invention has not been determined. Products as long as 9.4 kilobases have been obtained using test genes and the variant method. If difficulty is encountered in amplifying longer regions, the time permitted for intrastrand annealing may be increased. Alternately, the use of primers which are homologous to a portion of the known region of the cancer-associated gene very near the unknown region may be useful.

The variant panhandle PCR method of the invention may have advantages relative to the basic panhandle PCR method of the invention. In the basic panhandle PCR method, template-directed primer extension of the loop-forming oligonucleotide completes formation of the handle. This has the disadvantage of frequently creating a long complementary sequences, which can impede PCR initiation. Long complementary sequences are not created during the polymerase extension step in the variant panhandle PCR method, because the initial polymerase extension generates only a short complementary sequence that extends only as far as Primer 1. It has also been demonstrated that single primers inhibit PCR amplification of short products and amplify long target sequences with greater specificity. The variant panhandle PCR method is designed to use single primers very effectively. Single primer amplifications will not impede the amplification of products >1 kb. However, single primers also may be used in the basic panhandle PCR method. Also, the variant panhandle PCR method does not require a phosphorylated polynucleotide. For these reasons, the variant panhandle PCR method of the invention may have advantages relative to the basic panhandle PCR method of the invention in certain circumstances. Both are advantageous with respect to conventional cloning methods.

Another Embodiment of the Panhandle PCR Methods of the Invention

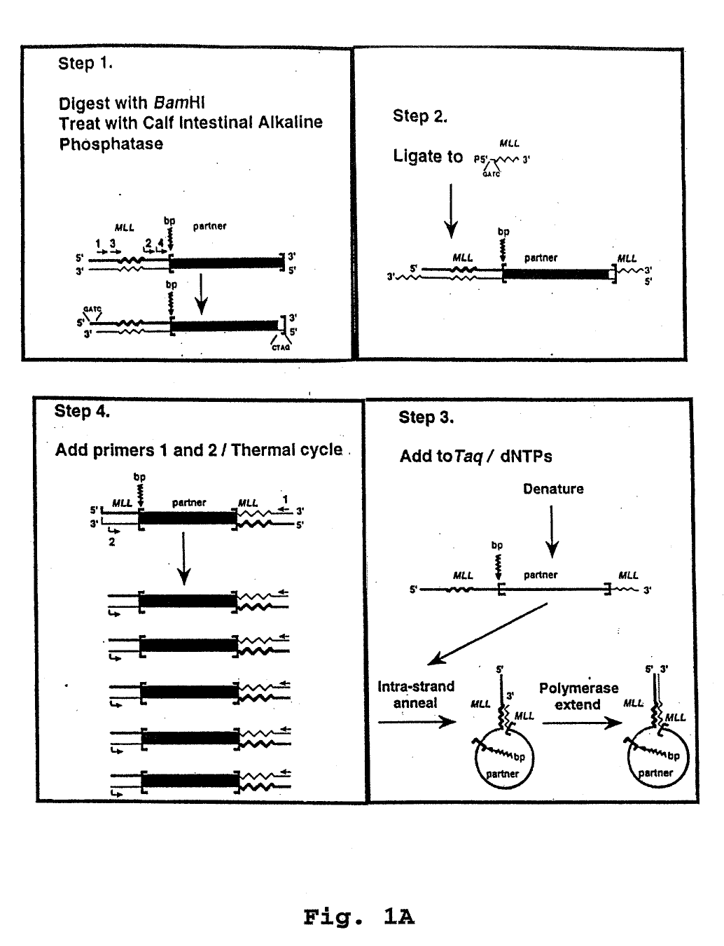

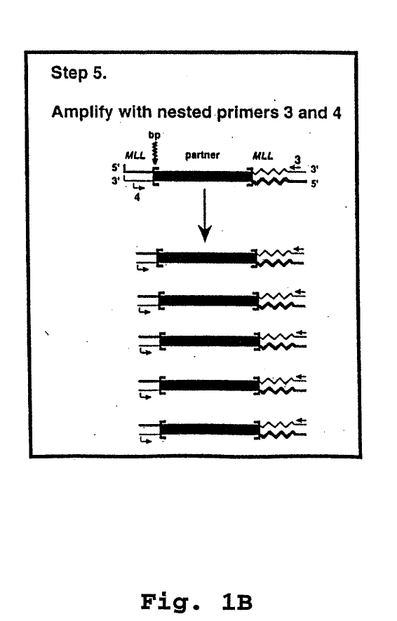

In another embodiment represented in FIG. 1, the basic panhandle PCR method of the invention is performed as follows.

High molecular weight genomic DNA is isolated from a patient afflicted with leukemia by ultracentrifugation on 4 molar GITC/5.7 molar CsCl gradients as described (Felix et al., 1990, J. Clin. Oncol. 8:431-442). Before performing panhandle PCR, genomic DNA from the patient is examined by Southern blot analysis for rearrangement of the 8.3 kilobase BamHI fragment that comprises the bcr of MLL, as described (Felix et al., 1995, Blood 85:3250-3256). Size(s) of any rearrangement(s) detected by Southern blot analysis indicates the possible anticipated approximate size of panhandle PCR products.

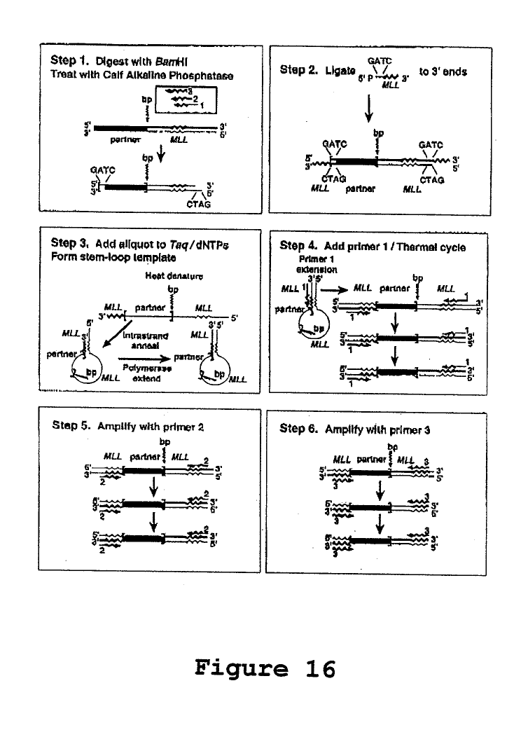

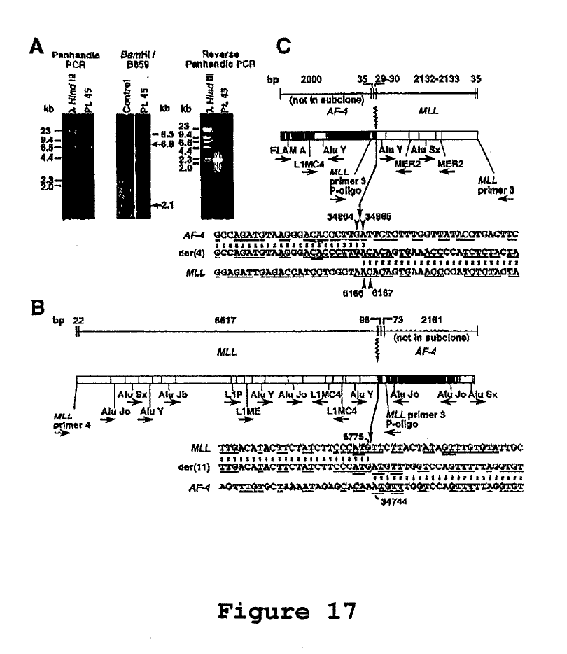

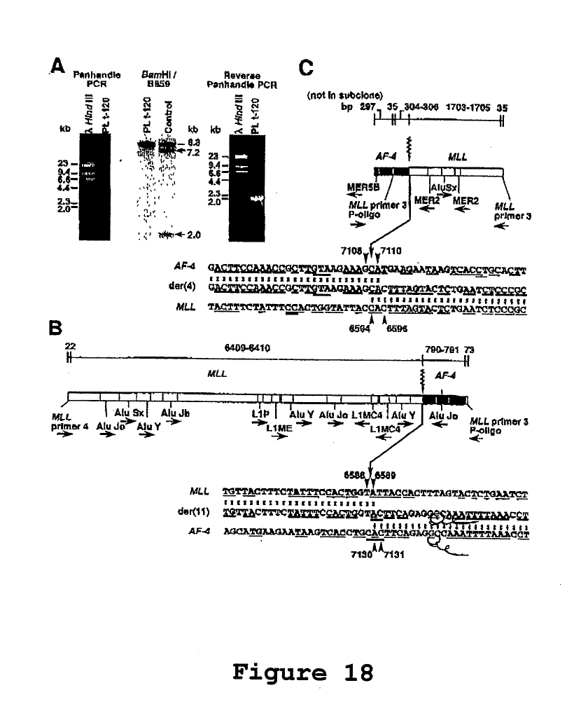

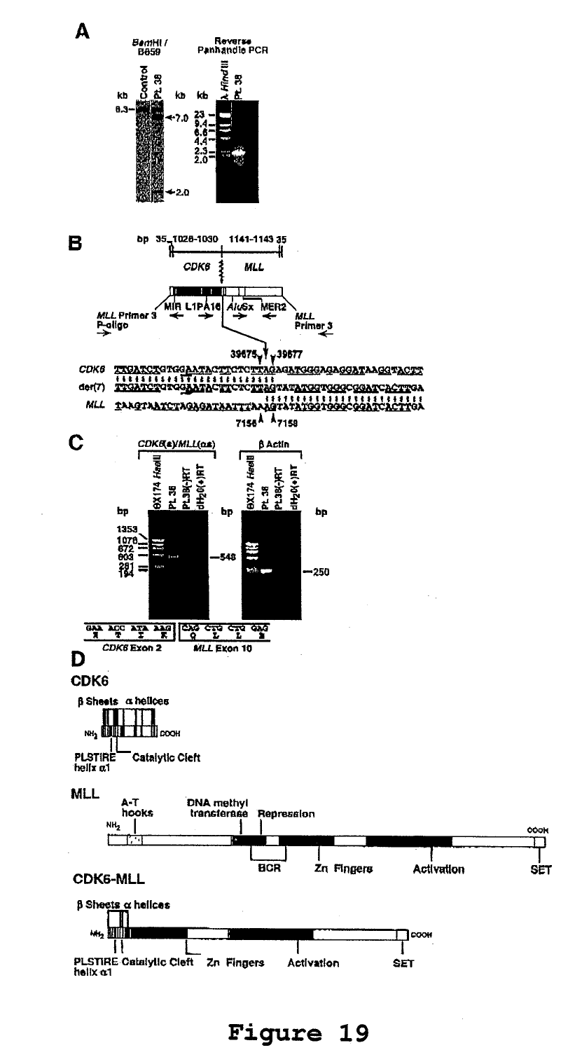

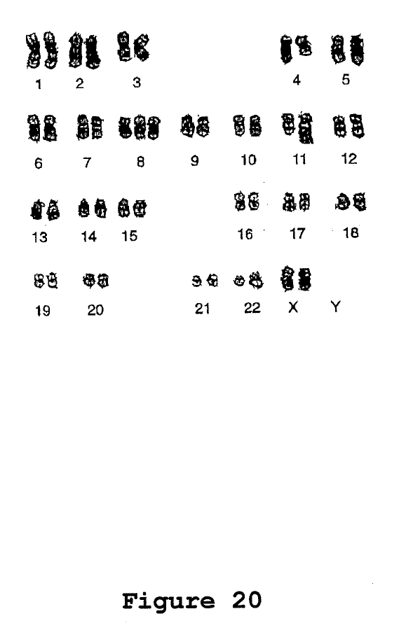

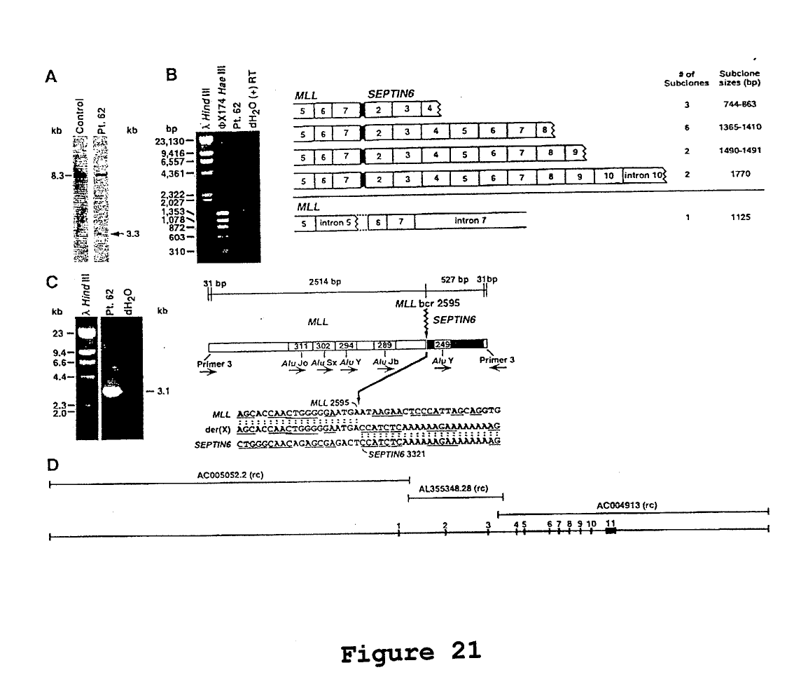

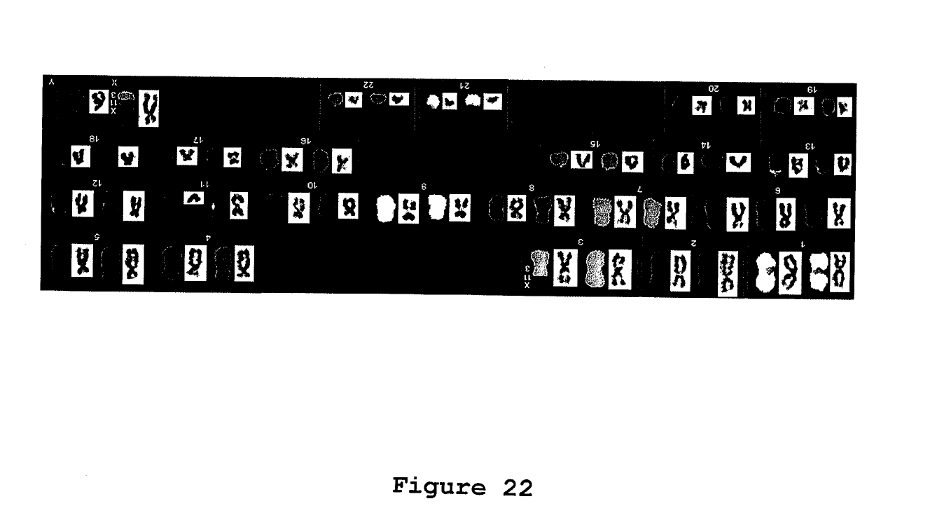

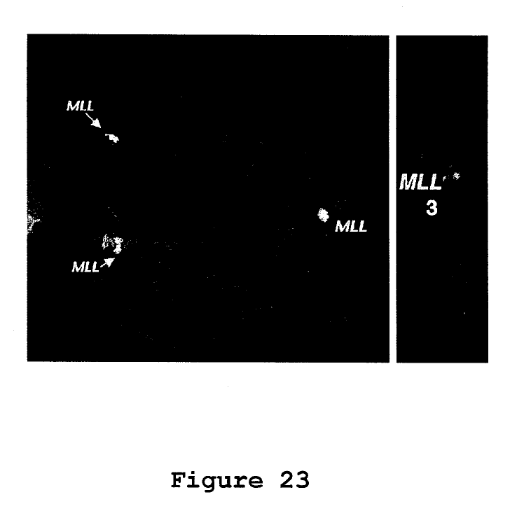

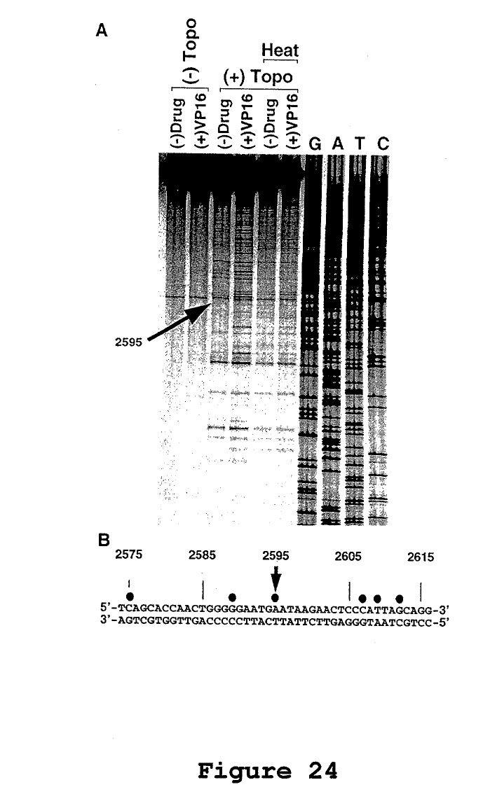

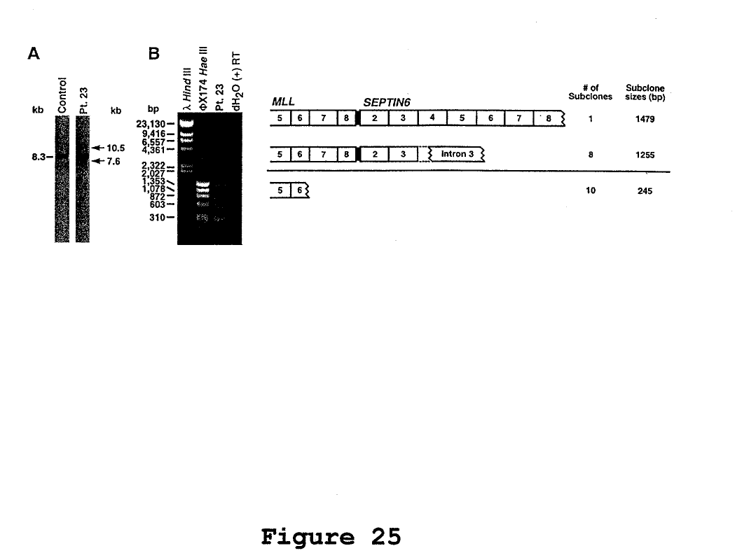

The method represented in FIGS. 1 and 16 amplifies the breakpoint region of the der(11) chromosome, and is described in this embodiment in five steps. These five steps are first described generally, after which a specific protocol is described.

The first step represented in FIGS. 1 and 16 concern the generation of the template polynucleotide. A genomic DNA fragment is treated with the restriction endonuclease BamHI to generate a genomic DNA which has overhanging 5′-ends and which comprises a known region of the MLL gene and an unknown region of a translocation partner gene flanking the known region. For leukemias associated with MLL gene translocations, BamHI is the most appropriate restriction endonuclease for use in the panhandle PCR methods of the invention, because virtually all MLL genomic breakpoint regions are located on the same 8.3 kb BamHI restricted genomic DNA fragment. The genomic DNA fragment is treated with calf intestinal alkaline phosphatase to prevent religation in Step 2.

The purpose of Steps 2 and 3 is to form the “handle” of the panhandle structure using the template polynucleotide. Formation of the handle involves ligating DNA complementary to a known sense region of MLL to the 3′-end of the unknown region of the sense strand and forming an intrastrand loop comprising the breakpoint region of MLL and unknown translocation partner DNA. Step 2, as represented in FIG. 1, involves ligation of a single stranded 5′-phosphorylated loop-forming oligonucleotide to the 3′-ends of the genomic DNA fragment. The four-nucleotide 5′-end of the loop-forming oligonucleotide is complementary to the 5′-overhanging region of the BamHI-digested genomic DNA fragment. The 3′-end of the loop-forming oligonucleotide is complementary to a first portion of the known region of the sense strand of MLL comprising exon 5, which is located in the bcr of MLL. The sense strand (the top strand in Step 2 of FIG. 1) is the template polynucleotide represented in Step 3.

Formation of the handle is completed in Step 3 by intrastrand annealing of the loop-forming oligonucleotide to the first portion of the known region, and by subjecting the resulting panhandle structure to extension. The panhandle structure is subjected to extension by adding the polynucleotide to a reaction mixture comprising DNA polymerase, dNTPs, and PCR reaction buffer. The reaction mixture is preheated to 80° C. before the addition of the panhandle structure to the reaction mixture in order to prevent non-specific annealing and polymerization. After addition of the panhandle structure, the reaction mixture is heated to 94° C. for 1 minute to generate single-stranded polynucleotide. Intrastrand annealing of the loop-forming oligonucleotide to the first portion of the known region and template-directed polymerase extension of the recessed 3′-end of the panhandle structure are effected by subjecting the mixture to a 2 minute ramp to 72° C. and incubation of the reaction mixture at 72° C. for 30 seconds, whereby the handle of the panhandle structure is extended.

In steps 4 and 5, as represented in FIGS. 1 and 16, primers homologous with the sense strand of portions of exon 5 of MLL are used to amplify the breakpoint region and the unknown translocation partner DNA. The positions and orientations of the primers with respect to the ligated polynucleotide are shown in step 1 of FIGS. 1 and 16. Step 4 comprises subjecting the panhandle structure generated in step 3 to PCR in the presence of primers 1 and 2. Primer 1 is homologous to a portion of the sense strand of MLL exon 5 located 5′ with respect to the first portion. Primer 2 is homologous to a portion of the sense strand of MLL exon 5 located between the 3′-end of the first portion and the translocation breakpoint. A nested PCR reaction is performed in step 5, in the presence of internal primers 3 and 4 to yield an amplified polynucleotide product which comprises the unknown region.

A specific protocol corresponding to the embodiment of the basic panhandle PCR method represented in FIGS. 1 and 16 is now described. Step numbers refer both to FIGS. 1 and 16 and to the immediately preceding discussion.

Step 1. BamHI digestion and calf intestinal alkaline phosphatase (CIAP) treatment

1. About 5 micrograms of genomic DNA obtained from a patient afflicted with leukemia is digested to completion at 37° C. for two hours in a digestion mixture comprising an appropriate buffer containing bovine serum albumin and 40 units (8 units per microgram) of BamHI (New England Biolabs, Beverly, Mass.). The reaction volume is 100 microliters. Genomic DNA fragments having 5′-overhanging regions are thereby generated.

2. The genomic DNA fragments are dephosphorylated by adding 0.05 unit of calf intestinal alkaline phosphatase (Boehringer Mannheim Biochemicals, Indianapolis, Ind.) to the digestion mixture. A 100 microliter CIAP stock solution comprising 0.01 unit per microliter of CIAP is prepared by diluting the 1 unit per microliter CIAP preparation supplied by the manufacturer 100-fold in TE buffer, which comprises 10 millimolar Tris-HCl and 1 millimolar EDTA. 5 microliters of the CIAP stock solution is added to the digestion mixture, and the mixture is incubated at 37° C. for 30 minutes.

3. CIAP-treated genomic DNA fragments are purified by glass bead extraction using a GENECLEAN III kit (BIO 101, Inc., La Jolla, Calif.) according to the manufacturer's instructions for 5 micrograms of genomic DNA in order to eliminate the protein. Purified DNA fragments are eluted in a final volume of 50 microliters of TE buffer. 25 microliters of the eluted fragments are stored at −20° C. for later use as an unligated control.

Step 2. Ligation of single-stranded 5′ phosphorylated loop-forming oligonucleotide to the 3′ ends of the genomic DNA fragments

The sequence of the 5′ phosphorylated loop-forming oligonucleotide useful for amplification of the translocation breakpoint region of the der(11) chromosome is 5′-GATCGAAGCT GGAGTGGTGG CCTGTTTGGA TTCAGG-3′ (SEQ ID NO: 4). The 32-nucleotide 3′-end of this 5′ phosphorylated loop-forming oligonucleotide is complementary to nucleotides 92-123 of the bcr of MLL, in MLL exon 5. The four-nucleotide-residue 5′-end of this 5′ phosphorylated loop-forming oligonucleotide is complementary to the 5′-overhanging region of the genomic DNA fragments, and is designed such that it does not reconstitute the BamHI site upon ligation of the loop-forming oligonucleotide to the genomic DNA fragment.

1. The 5′-phosphorylated loop-forming oligonucleotide is suspended in distilled water at a final concentration of 0.25 micrograms per microliter.

2. Reagents are added to a container to generate a ligation mixture having a final volume of 50 microliters. The ligation mixture comprises 16.9 microliters of distilled water, 25 microliters (2.5 micrograms) of phosphatase-treated genomic DNA fragments suspended in TE buffer, 2.1 microliters (516 nanograms) of the 5′-phosphorylated loop-forming oligonucleotide, 5 microliters of 10×ligase buffer (Boehringer Mannheim Biochemicals, Indianapolis, Ind.), and 1 microliter of a solution comprising 1 Weiss Unit per microliter of T4 DNA ligase (Boehringer Mannheim Biochemicals, Indianapolis, Ind.). The ligation mixture is incubated overnight at 4° C. or, alternately, at 17° C., to generate oligonucleotide-ligated DNA fragments. The 516 nanograms of loop-forming oligonucleotide represents an approximately 50-fold molar excess with respect to the genomic DNA fragments.

3. The oligonucleotide-ligated DNA fragments are purified using a GENECLEAN III kit (BIO 101, Inc., La Jolla, Calif.) according to the manufacturer's directions. The oligonucleotide-ligated DNA fragments are eluted in a final volume of 25 microliters of TE buffer.

Step 3. Addition of DNA to Taq/dNTP mixture, Denaturation Intrastrand annealing, Panhandle formation, and Polymerase extension

1. 25 microliters of a 2×PCR reagent is prepared by adding to a container 2.5 units (0.75 microliter) of a Taq/Pwo DNA polymerase mixture (Expand Long Template PCR System, Boehringer Mannheim Biochemicals, Indianapolis, Ind.), 0.7 microliters of a 1:1:1:1 nucleoside mixture comprising 25 micromolar each dATP, dCTP, dGTP, and dTTP, 5 microliters of 10 PCR reaction buffer (Boehringer Mannheim Biochemicals, Indianapolis, Ind.), and 18.55 microliters of distilled water. The 2×PCR reagent may be prepared as a bulk cocktail, pre-aliquoted, and stored at −20° C. for future use.

2. The oligonucleotide-ligated DNA fragments are subjected to intrastrand annealing and extension by preparing a reaction mixture comprising 18 microliters of distilled water, 25 microliters of 2×PCR reagent in a 500 microliter thin-wall tube (Perkin-Elmer). One drop (circa 50 microliters) of mineral oil is layered atop the reaction mixture. To prevent non-specific annealing and polymerization, the tube is pre-heated to 80° C. in a thermal cycler.

3. A 200 nanogram aliquot (2 microliters) of the suspension of oligonucleotide-ligated DNA fragments is added to the pre-heated reaction mixture. After addition of the DNA suspension, the reaction mixture contains 2.5 units of Taq/Pwo DNA polymerase mix (Expand Long Template PCR System, Boehringer Mannheim Biochemicals, Indianapolis, Ind.), 385 micromolar each dNTP (Expand Long Template PCR System, Boehringer Mannheim Biochemicals, Indianapolis, Ind.), and PCR reaction buffer at 1.1 final concentration in a volume of 45 microliters. The reaction mixture is heated at 94° C. for 1 minute to dissociate the oligonucleotide-ligated DNA fragments. A negative control reaction mixture comprises all of the reaction mixture reagents, except that 200 nanograms (2 microliters) of the unligated control DNA is used in place of the oligonucleotide-ligated DNA fragments. A reagent control reaction mixture comprises all of the reaction mixture reagents, but does not comprise DNA.

4. Intrastrand annealing of the loop-forming oligonucleotide to the complementary sequence of the first portion of the known region to form a panhandle structure and polymerase extension of the recessed 3′-end of the panhandle structure are effected by following the 94° C. heat denaturation step with a two minute ramp of the reaction mixture temperature to 72° C. and incubation of the reaction mixture at 72° C. for 30 seconds.

5. Maintain the reaction mixture at 80° C. before addition of the PCR primers in Step 4 to prevent priming at low stringency and generation of nonspecific products.

Step 4. Addition of MLL primers 1 and 2 and thermal cycling

1. The nucleotide sequence of MLL primer 1 is 5′-TCCTCCACGA AAGCCCGTCG AG-3′ (SEQ ID NO: 5), and the nucleotide sequence of MLL primer 2 is 5′-TCAAGCAGGT CTCCCAGCCA GCAC-3′ (SEQ ID NO: 6). With the reaction mixture maintained at 80° C., add 12.5 picomoles of each primer in a volume of 2.5 microliters to the reaction mixture to yield a first PCR mixture. These additions result in concentrations in the 50 microliter first PCR mixture of 350 micromolar for each dNTP and 1× for PCR reaction buffer. In a variation of this embodiment, primer 2 has a nucleotide added to its 5′-end that was not homologous with the known region of the sense strand of MLL. This is a precaution to prevent short-circuiting of PCR in the first PCR mixture if using Taq DNA polymerase alone. Short-circuiting could occur by annealing of the 3′-end of one strand of a short nonspecific PCR product to the template polynucleotide. The necessity of this precaution has not been tested. Results obtained using a similar method involving long-range PCR reagents including a DNA polymerase having 3′ exonuclease activity suggests that this precaution is unnecessary.

2. If Southern blot analysis information is available, then that information can be used to determine the duration of the elongation segment in the PCR reaction (using as a rule of thumb that 1 minute should be allowed per kilobase). To amplify products 8.3 kilobases and 7 kilobases in length, the following conditions have been used. The initial denaturation was performed at 94° C. for 1 minute. Ten cycles were performed by maintaining the first PCR mixture at 94° C. for 10 seconds and at 68° C. for 7 minutes. Twenty cycles were performed by maintaining the first PCR mixture at 94° C. for 10 seconds and at 68° C. for 7 minutes, wherein the period during which the mixture was maintained at 68° C. was incremented 20 seconds per cycle. A final elongation was performed at 68° C. for 7 min. It is understood that shorter products can be amplified using shorter, as well as longer, elongation times.

Step 5. Perform nested PCR using internal primers 3 and 4

1. The nucleotide sequence of MLL internal primer 3 is 5′-AGCTGGATCC GGAAAAGAGT GAAGAAGGGA ATGTCTCGG-3′ (SEQ ID NO: 7), and the nucleotide sequence of MLL internal primer 4 is 5′-AGCTGGATCC GTGGTCATCC CGCCTCAGCCAC-3′(SEQ ID NO: 8). Underlined sequences are BamHI restriction endonuclease sites. A second PCR mixture is prepared by combining 25 microliters of 2×PCR reagent, 19 microliters of distilled water, 2.5 microliters (12.5 picomoles) of each of MLL internal primers 3 and 4, and a 1 microliter aliquot of the first PCR mixture. One drop (circa 50 microliters) of mineral oil is layered atop the second PCR mixture. The second PCR mixture is subjected to the same PCR conditions as was the first PCR mixture.

3 microliters of the second PCR mixture is visualized on an ethidium-stained minigel. Detection of a product having the same approximate size as the BamHI fragment detected by genomic Southern blot analysis is an indication that the amplified products obtained following Step 5 comprise the known sequence from MLL flanked by the unknown partner DNA.

Subcloning and Sequencing of the Products of Panhandle PCR

Each of MLL internal primers 3 and 4 comprises a BamHI restriction sites which is useful for subcloning. The amplified products obtained following Step 5 are isolated using an agarose gel and subcloned to permit sequencing of the translocation breakpoint region, the unknown region, or both.

To validate the results, validating primers may be designed from sequences of the subcloned products of panhandle PCR. These validating primers may encompass the translocation breakpoint region. If such validating primers are used to amplify genomic DNA obtained from the patient afflicted with leukemia, direct genomic sequencing may be performed, and the results obtained using panhandle PCR may thereby be confirmed. RT-PCR may also be used to validate results obtained by panhandle PCR methods.

This embodiment of the panhandle PCR methods has been used to clone three MLL genomic breakpoint regions, amplifying polynucleotides from about 2.5 kilobases to about 8.3 kilobases in length. Application of this embodiment of the methods in three cases of infant ALL and in two treatment-related leukemias identified the respective MLL genomic breakpoint regions and previously uncharacterized intronic sequences in the partner genes. In two of the five cases, the karyotype did not suggest the chromosomal location of the translocation partner.

Panhandle PCR is a technical advance over prior methods of investigating the molecular pathogenesis of leukemias associated with MLL gene translocations. This embodiment of the panhandle PCR methods of the invention is practical in cases where the amount of genomic DNA is limited. Panhandle PCR is a definitive PCR approach for identifying additional new partner genes of MLL and for amplifying the translocation breakpoint regions of other genes wherein the partner gene is undetermined.

Subcloning of the Products of Panhandle Variant PCR by Recombination PCR

Recombination PCR uses E. coli itself to mediate DNA recombination (Jones et al., 1991, BioTechniques 10:62-66). The observation that DNA ends containing short regions of homology can undergo intra- and intermolecular recombination in vivo in E. coli, including RecA(−) strains routinely used for subcloning, led to the development of recombination PCR (Jones et al., 1991, BioTechniques 10:62-66). A sample protocol which can be used with either of the panhandle PCR methods of the invention is now described.

First, 0.5 micrograms of pUC 19 (Gibco BRL) is linearized by digestion with 10 units HindIII (Gibco BRL). A 2 nanogram aliquot of the restriction enzyme-digested plasmid template is amplified in a 50 microliter PCR reaction containing 1.25 units Amplitaq DNA polymerase (Perkin Elmer, Norwalk, Conn.), 12.5 picomoles of each primer, 200 micromolar of each dNTP, and 1×PCR reaction buffer (Perkin Elmer) to generate a linearized plasmid having ends complementary to the ends of the product of panhandle PCR product to be inserted. The sequences of the primers used to amplify the HindIII-digested pUC 19 may be, for example, 5′-TCCCTTCTTC ACTCTTTTCC TCGATGGCGT AATCATGGTC ATAGC-3′ (SEQ ID NO: 19) and 5′-TCCCTTCTTC ACTCTTTTCC TCGACATGCC TGCAGGTCGA CTCTAGAG-3′ (SEQ ID NO: 20). After initial denaturation at 94° C. for 1 minute, twenty five cycles are performed, wherein the reaction mixture is maintained at 94° C. for 30 seconds, at 50° C. for 30 seconds, and at 72° C. for 2 minutes, 42 seconds, followed by a final elongation at 72° C. for 7 minutes.

The products from PCR amplification of the plasmid and from panhandle variant PCR may purified using a Geneclean III kit (Bio 101, Inc., La Jolla, Calif.) according to the manufacturer's instructions and resuspended in 10 microliters of elution buffer provided in the kit (Bio 101, Inc.). 2.5 microliters of the purified PCR products from amplification of the plasmid and 2.5 μl of the purified panhandle PCR products are combined and added to 50 microliters of MAX Efficiency DH5a Competent Cells (Life Technologies, Gaithersburg, Md.) to undergo in vivo recombination. The transformation procedure is as described in the manufacturer's instructions (Life Technologies), except that the entire 1 milliliter reaction is plated. Individual transformants are grown overnight in 4 milliliters of Luria broth containing 100 micrograms per milliliter ampicillin.

PCR may be used to identify recombinant plasmids containing products of panhandle PCR. 2 microliter aliquots of the saturated 4 milliliter cultures are amplified in PCR reactions containing 0.5 microliters (1.75 units) of Taq/Pwo DNA polymerase mix, 350 micromolar of each dNTP, 1×Expand Buffer 1 (Expand Long Template PCR System, Boehringer Mannheim) and 12.5 picomoles of a primer, such as the first oligonucleotide of the variant panhandle PCR method. The PCR conditions are the same as those for panhandle variant PCR, as described herein. Analysis of the products by agarose gel electrophoresis identifies those transformants containing the recombinant plasmid DNA of interest. Desired transformants are grown in 25 milliliter cultures for plasmid preparation and automated sequencing.

Method of Identifying a Translocation Partner

The invention also includes a method of identifying a translocation partner of a cancer-associated gene. It is understood that the translocation partner may be a gene other than the cancer-associated gene or a portion of the cancer-associated gene which is duplicated. This identification method of the invention is performed as follows.

An unknown region flanking a cancer-associated gene is amplified using one of the panhandle PCR methods of the invention. After the unknown region is amplified, a portion of a human gene homologous with the unknown region is identified, whereby that human gene is identified as the translocation partner. Numerous methods are known whereby a portion of a human gene homologous with an amplified portion of a template polynucleotide may be identified. The choice of method used to identify such a homologous human gene is not critical. By way of example, the nucleotide sequence of the unknown region may be determined and then compared with the nucleotide sequence of a cloned or characterized human gene. The human gene may be one which has a sequence listed in a database of human gene sequences. Further by way of example, the nucleotide sequence of the unknown region may be compared with the nucleotide sequence of a human gene by contacting a test polynucleotide with a control polynucleotide derived from the human gene. The test polynucleotide may be selected from the group consisting of a polynucleotide homologous with the unknown region and a polynucleotide complementary to the unknown region. If the test polynucleotide is capable of annealing with the control polynucleotide with high stringency, then the human gene is either homologous with or complementary to the unknown region of the template polynucleotide. Either way, the translocation partner is identified as at least a portion of the human gene.

A Kit for Panhandle PCR Analysis of Cancer-associated DNA Sequences

The invention further includes a kit useful for performing the panhandle PCR methods of the invention. The kit of the invention for performing the basic panhandle PCR method comprises an oligonucleotide and a first primer. The oligonucleotide is complementary to a known region of the sense strand of a cancer-associated DNA sequence. The first primer is homologous with the known region. The oligonucleotide and first primer are used in the basic panhandle PCR method as described herein, the oligonucleotide being the loop-forming oligonucleotide of the basic panhandle PCR method.

The kit of the invention for performing the variant panhandle PCR method also comprises an oligonucleotide and a first primer. The oligonucleotide is homologous with a known region of the sense strand of a cancer-associated DNA sequence. The primer is homologous with the known region. The oligonucleotide and first primer are used in the variant panhandle PCR method as described herein, the oligonucleotide being the first oligonucleotide of the basic panhandle PCR method, and the first primer being the pre-template polynucleotide of that method, the first primer of that method, or both.

In either kit, the cancer-associated DNA sequence may, for example, be MLL. The known region may, for example, be selected from the group consisting of a portion of the breakpoint cluster region of MLL, a portion of MLL which flanks the breakpoint cluster region of MLL, a portion of an intron of MLL, and a portion of an exon of MLL such as exon or exon 11.

The kit of the invention may further comprise an internal primer, wherein the internal primer is nested with respect to the first primer, and wherein the internal primer is selected from the group consisting of a primer homologous with the known region of the sense strand. For example, the internal primer may be a primer homologous with a portion of the known region of the sense strand near the end of the known region that is nearer an unknown region which flanks the known region. The kit may, of course, comprise a plurality of internal primers.

The kit of the invention may also comprise a second primer, wherein when the first primer is homologous with the known region of the sense strand, said second primer is homologous with a portion of the known region which is located within the “pan” portion of the panhandle structure generate using the kit of the invention.

The kit of the invention may comprise an internal primer which is nested with respect to each of the first primer and the second primer, and wherein the internal primer is homologous with the known region of the sense strand.

In addition to the various primers described herein, the kit of the invention may further comprise one or more of a restriction endonuclease, at least one reagent for ligating an oligonucleotide to a DNA strand obtained from a human patient, at least one reagent for extending a polynucleotide, or at least one reagent for performing PCR. The kit of the invention may also comprise one or more recombination PCR primers, as described herein to amplify the linearized plasmid if recombination PCR-based subcloning is desired. Examples of recombination PCR primers which may be included in the kit include the following two primers:

(SEQ ID NO:21)

5′-ACATTCCCTT CTTCACTCTT TTCCTGGCGT AATCATGGTC
ATAGC-3′ and

(SEQ ID NO:22)

5′-GTGGCTGAGG CGGGATGACC ACCATGCCTG CAGGTCGACT
C-3′

Reagents useful for ligating an oligonucleotide to a DNA strand are well known and include, for example, T4 DNA ligase and buffers in which T4 DNA ligase is known to be enzymatically active. Reagents useful for template-directed polynucleotide extension are also well known and include, for example, Taq DNA polymerase, Pwo DNA polymerase, nucleoside triphosphates, and appropriate buffers. Reagents useful for performing PCR are likewise well known and include, for example, Taq DNA polymerase, Pwo DNA polymerase or another proof-reading enzyme, nucleoside triphosphates, and appropriate buffers.

### EXAMPLES

The invention is now described with reference to the following Examples. These Examples are provided for the purpose of illustration only and the invention should in no way be construed as being limited to these Examples, but rather should be construed to encompass any and all variations which become evident as a result of the teaching provided herein.

### Example 1

Panhandle PCR: A Technical Advance in MLL Genomic Breakpoint Region Cloning

Known panhandle PCR methods (e.g. Jones et al., 1992, Nucl. Acids Res. 20:595-600; Jones, 1995, PCR Meth. Applicat. 4:S195-S201) were adapted for use in cloning a MLL genomic breakpoint region on a der(11) chromosome obtained from a three month old female infant patient afflicted with ALL. Karyotype analysis of the patient was technically unsuccessful. As described in this Example, a basic panhandle PCR method was adapted to facilitate cloning of the 11q23 genomic translocation breakpoint.

A 5′-phosphorylated loop-forming oligonucleotide, which is complementary to a portion of MLL, was ligated to translocation partner DNA located 3′ with respect to the breakpoint. It was thereby possible to generate a template polynucleotide capable of forming a panhandle structure wherein the intrastrand loop of the panhandle structure comprised the translocation breakpoint region, including the unknown partner DNA. The duplex “handle” portion of the panhandle structure comprised a portion of the template polynucleotide homologous with a known region of MLL and a region complementary to that portion. Primers derived from MLL were used to amplify the breakpoint region, including the unknown partner DNA.

The materials and methods used in the experiments presented in this Example are now described.

The Infant Patient

The three month old infant patient presented with a white blood cell count (WBC) of 399×10⁹ cells per liter and with a large extramedullary tumor burden typical of infant ALL. Consistent with early B lineage ALL, the patient's bone marrow was replaced by lymphoblasts of French-American-British L1 morphology that expressed tdt, CD34, HLA DR and CD19, but not CD10, CD20, or myeloid antigens.

Before performing panhandle PCR, Southern blot analysis of genomic DNA from the infant patient afflicted with ALL was performed to detect rearrangement of the 8.3 kb BamHI fragment that comprises the bcr of MLL. When such a rearrangement was detected, the size of the rearranged fragment was used as an approximation of the expected size of the polynucleotide to be amplified by panhandle PCR.

A Basic Panhandle PCR Method was Performed as Follows.

Step 1. Genomic DNA was digested using restriction endonuclease BamHI, yielding genomic DNA fragments each having a 5′-overhanging region at each end. One of these fragments comprises a known region of MLL, the bcr of MLL, and, if the fragment was obtained from a patient in whom a translocation event associated with MLL had occurred, translocation partner DNA. The fragments were treated with calf intestinal alkaline phosphatase to prevent religation in Step 2.

Step 2. The purpose of Steps 2 and 3 was to form the handle portion of a panhandle structure. Step 2 involved ligation of a single-stranded 5′-phosphorylated loop-forming oligonucleotide to each of the 3′-ends of the genomic DNA fragments generated in Step 1. The loop-forming oligonucleotide had a four-nucleotide 5′-overhanging region which was complementary to the 5′-overhanging regions of the fragments. The 3′-end of the loop-forming oligonucleotide was complementary to a first portion of MLL, the first portion comprising a portion of exon 5, which is within the bcr of MLL. The sense strand of the fragment was used to generate the template polynucleotide in Step 3.

Step 3. Formation of the handle portion of the panhandle structure was completed in Step 3 by intrastrand annealing of the loop-forming oligonucleotide with the first portion of the template polynucleotide and subsequent polymerase extension of the recessed 3′-end of the duplex region of the template polynucleotide. At this point, the intrastrand loop portion of the panhandle structure comprised the translocation breakpoint region and unknown partner DNA, and the handle portion of the panhandle structure comprised a known region of the template polynucleotide having known (MLL) sense sequence and sequence complementary to this known region.

Steps 4 and 5. Because the template polynucleotide comprised regions of known sequence at each end, it was possible to use primers derived from MLL to amplify a portion of the template polynucleotide. The primers which were used were in the sense orientation with respect to exon 5 of MLL. Nested PCR primers were used in Step 5 to enhance the yield of amplified template polynucleotide product. This method has been used to clone five MLL genomic breakpoint regions.

Further Details of This Procedure Were as Follows:

The basic panhandle PCR method described in this example was analogous to the method depicted in FIG. 1, which is referred to in this Example for the purpose of illustration only.

In step 1 in FIG. 1, 5 micrograms of genomic DNA was digested to completion using 40 units of BamHI (New England Biolabs, Beverly, Mass.) to generate genomic DNA fragments having 5′-overhanging regions. The genomic DNA comprised a known region of MLL and an unknown region flanking the known region, the unknown region comprising the translocation partner DNA. The genomic DNA fragments were treated with 0.05 units of calf intestinal alkaline phosphatase (Boehringer Mannheim Biochemicals) at 37° C. for 30 minutes to prevent religation in step 2. The genomic DNA fragments were purified using a GENECLEAN II® kit (BIO 101, Inc., La Jolla, Calif.).

In step 2 in FIG. 1, a single-stranded 5′-phosphorylated loop-forming oligonucleotide was ligated to the 3′-end of each genomic DNA fragment strand. The sequence of the loop-forming oligonucleotide was 5′-GATCGAAGCT GGAGTGGTGG CCTGTTTGGA TTCAGG-3′ (SEQ ID NO: 4). The four-nucleotide-residue 5′-end of the loop-forming oligonucleotide was complementary to the 5′-overhanging region of the genomic DNA fragments, and does not reconstitute the BamHI site upon ligation to an individual genomic DNA fragment. The thirty-two nucleotides of the 3′-end of the loop-forming oligonucleotide were complementary to nucleotide positions 92-123 of MLL exon 5, which is within the bcr of MLL. The 50 microliter ligation reaction mixture comprised 2.5 micrograms of genomic DNA fragments, a 50-fold molar excess of the loop-forming oligonucleotide, 1 Weiss Unit of T4 DNA ligase (Boehringer Mannheim), and 1×ligase buffer (Boehringer Mannheim). Ligations were performed overnight at 4° C. to generate template polynucleotide. The template polynucleotide was purified using a GENECLEAN II® kit (BIO 101, Inc., La Jolla, Calif.).

In step 3 in FIG. 1, the panhandle structure was generated. A 200 nanogram aliquot of template polynucleotide was added to an extension mixture comprising 2.5 U Taq/Pwo DNA polymerase mix, 385 μM each dNTP, and PCR reaction buffer at 1.1×final concentration in a total volume of 45 microliters (Expand Long Template PCR System, Boehringer Mannheim Biochemicals, Indianapolis, Ind.). The extension mixture was preheated to 80° C. before addition of the template polynucleotide to prevent non-specific annealing and polymerization. The extension mixture was then maintained at 94° C. for 1 minute to denature the template polynucleotide. The sense strand with respect to MLL was used as the template polynucleotide (represented by the top strand in FIG. 1). Intrastrand annealing of the loop-forming oligonucleotide to the complementary sequence in the known (MLL) portion of the template polynucleotide yielded the panhandle structure. Polymerase extension of the free 3′-end of the loop-forming oligonucleotide was accomplished by a 2 minute ramping of the extension mixture temperature to 72° C. to complete formation of the handle portion of the panhandle structure.

In step 4 in FIG. 1, primers homologous with known portions of MLL were added to the extension mixture and thermal cycling was performed. MLL primer 1 (5′-TCCTCCACGA AAGCCCGTCG AG-3′; SEQ ID NO: 5) and MLL primer 2 (5′-TCAAGCAGGT CTCCCAGCCA GCAC-3′; SEQ ID NO: 6) were added. 12.5 picomoles of each of these primers, each suspended in a volume of 2.5 microliters was added to the extension mixture to yield a first PCR mixture in which final concentrations in the 50 volume were 350 micromolar for each dNTP and 1× for PCR reaction buffer. Following a 1 minute initial denaturation period during which time the first PCR mixture was maintained at 94 for 1 minute Ten cycles were performed by maintaining the first PCR mixture at 94° C. for 10 seconds and at 68° C. for 7 minutes. Twenty cycles were performed by maintaining the first PCR mixture at 94° C. for 10 seconds and at 68° C. for 7 minutes, wherein the duration of this period was incremented 20 seconds per cycle. A final elongation was performed at 68° C. for 7 min.

In step 5 in FIG. 1, another PCR reaction was performed using nested MLL internal primers and a 1 microliter aliquot of the first PCR mixture as template to enhance the yield and specificity of the amplified polynucleotide product. Sequences of nested MLL internal primers 3 and 4 were 5′-AGCTGGATCC GGAAAAGAGT GAAGAAGGGA ATGTCTCGG-3′ (SEQ ID NO: 7) and 5′-AGCTGGATCC GTGGTCATCC CGCCTCAGCC AC-3′ (SEQ ID NO: 8), respectively. Conditions for nested PCR were the same as in Step 4.

The results of the experiments presented in this Example are now described.

Panhandle PCR Identified MLL Genomic Breakpoint Regions Comprising Unknown Partner DNA.

The basic panhandle PCR method was used to clone the translocation breakpoint region of the infant patient described herein, who was afflicted with ALL. Southern blot analysis of diagnostic marrow obtained from the three month old infant patient indicated that two MLL gene rearrangements had occurred the translocation breakpoint junction region, suggesting that chromosomal translocation had occurred. Southern blot analysis using BamHI-B859, XbaI-B859 and XbaI-SKV3 restriction enzyme-cDNA probe combinations indicated that the translocation breakpoint was located within the first 4464 nucleotide residues of the 8.3 kilobase bcr region of MLL. The translocation partner was unknown because no mitoses were available for karyotype analysis. Using the basic panhandle PCR method described herein, a predicted 8.3 kilobase amplification product was obtained from MLL genes in control DNA obtained from the infant patient's mother. Also using the basic panhandle PCR method described herein, a 7 kilobase amplification product was obtained from the der(11) chromosome of the leukemia of the infant patient. An 8.3 kilobase amplification product was also obtained from the normal MLL allele of the leukemia of the infant patient. The 7 kilobase amplification product was subcloned and the nucleotide sequences of three genomic subclones were determined.

The translocation breakpoint in the infant patient was identified at nucleotide position 3802 of the bcr of MLL, in MLL intron 8.

Subcloning and Sequencing of the Products of Panhandle PCR.

Polynucleotide products amplified using a panhandle PCR method described herein were separated on an agarose gel and subcloned into the BamHI site of pBluescript SK II® (Stratagene, Inc., La Jolla, Calif.) using standard methods. Automated nucleotide sequencing of three genomic subclones identified the MLL genomic breakpoint and the sequence of the unknown partner gene that was flanking MLL.

The predicted 8.3 kb panhandle PCR product from the normal MLL genes was obtained in control maternal DNA. Both a 7 kb product from the der(11) chromosome and an 8.3 kb product were obtained from the normal MLL allele in the leukemia (FIG. 2). The 7 kb product from the der(11) chromosome was subcloned and three individual genomic subclones were sequenced.

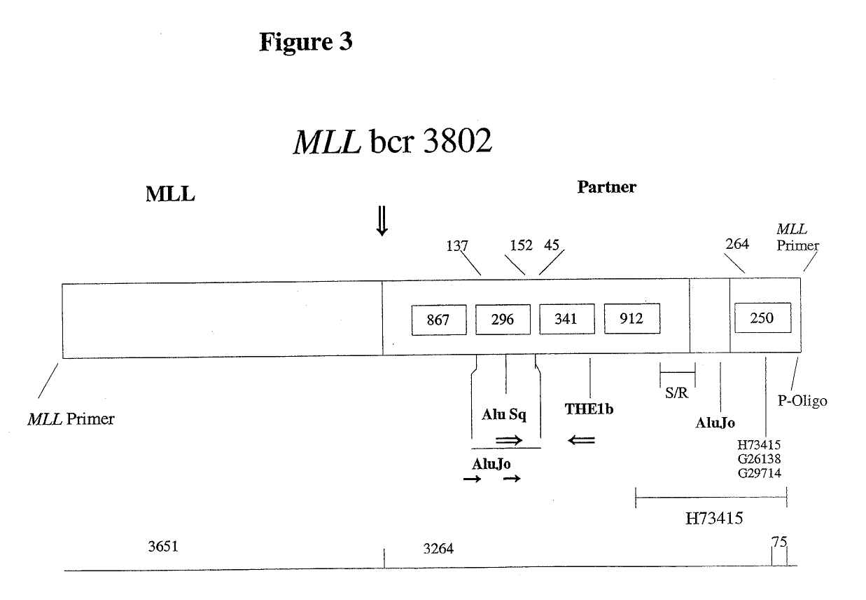

Automated sequencing of the 5′ bcr in subclone 34-1 from panhandle PCR identified the MLL genomic breakpoint at nucleotide 3802 in intron 8 and partial sequence of the partner DNA, as depicted in FIG. 3. Sequencing of two additional subclones from panhandle PCR verified the MLL genomic breakpoint at nucleotide 3802.

Repeat regions in the translocation partner gene were identified and masked using the Repeat Masker program available through the Washington University Human Genome Center (at the World Wide Web address, http://ftp.genome. washington.edu/cgi-bin/mrs/mrs_reg). Masked translocation partner gene sequence was submitted for BLAST analysis against the non-repetitive nucleotide database using the server at the Japanese Genome Center at Kyoto (at the World Wide Web address, http://www.genome.adjp/SIT/BLAST.html). The 3′-end of the unknown partner DNA was homologous to ESTs H73415, G26138, and G29714 in the database.

Direct Sequencing of the MLL Genomic Translocation Breakpoint Region.

Aliquots of genomic DNA obtained from cells of the infant patient were PCR amplified using validating primers derived from the polynucleotide product amplified by panhandle PCR. Primers which were used had nucleotide sequences 5′-GGGACTTTCT GTTGGTGGAA-3′ (SEQ ID NO: 9) and 5′-GAAACACCAG CAAACCAACC-3′ (SEQ ID NO: 10) or 5′-ATACATGTTG GGTGGCAGG-3′ (SEQ ID NO: 11) and 5′-GTCAAGGAAA GGTGGTATAT CTCA-3′ (SEQ ID NO: 12), and resulted in amplification of polynucleotides having lengths of 450 or 411 nucleotide residues, respectively. The PCR reaction mixtures each had a volume of 50 microliters and comprised 200 nanograms of genomic DNA, 0.5 unit Taq Gold® DNA polymerase (Perkin Elmer Cetus, Norwalk, Conn.), 250 micromolar of each dNTP, PCR reaction buffer at 1×final concentration (Perkin Elmer Cetus, Norwalk, Conn.), and 5 picomoles of each of two primers. After initial denaturation and Taq Gold® activation at 95° C. for 10 minutes, thirty-five cycles were performed by maintaining the PCR reaction mixture at 94° C. for 15 seconds, at 55° C. for 15 seconds, and at 72° C. for 1 minute. A final elongation was performed at 72° C. for 10 minutes. Amplified polynucleotide products were isolated from a 1.5% (w/v) agarose gel using a GENECLEAN II kit (BIO 101, Inc., La Jolla, Calif.). Approximately 100 nanograms of an amplified polynucleotide product was used for each direct nucleotide sequencing reaction. Sequencing was performed in both directions by automated methods. These results confirmed the MLL genomic breakpoint by an independent method.

Chromosomal Localization of the Translocation Partner Gene.

Panels of somatic cell hybrid DNAs and radiation hybrid DNAs were screened by PCR to identify the chromosomal location of the translocation partner gene. For the somatic hybrid screen, the 50 microliter PCR reaction mixtures comprised 500 nanograms of the DNA to be screened (Bios Laboratories, New Haven, Conn.), 1.25 units AmpliTaq® DNA polymerase, 200 micromolar of each dNTP, PCR reaction buffer at 1×final concentration (Perkin Elmer Cetus, Norwalk, Conn.) and 12.5 picomoles of each of two primers from the partner DNA. The region that the primers would amplify is designated S/R in FIG. 3. The primers which were used had nucleotide sequences 5′-CCTACACCCA GCCAAACTGT-3′ (SEQ ID NO: 13) and 5′-ATGGTACCAG AACAGGGCAG-3′ (SEQ ID NO: 14), and resulted in amplification of a polynucleotide having a length of 267 nucleotide residues. After initial denaturation at 94° C. for 9 minutes, thirty-five cycles were performed by maintaining the PCR reaction mixture at 94° C. for 1 minute, at 55° C. for 1 minute, and at 72° C. for 2 minutes. A final elongation was performed at 72° C. for 7 minutes. Human and hamster genomic DNA samples were used as controls. Twenty microliter aliquots of each PCR reaction mixture were electrophoresed in a 4% (w/v) Nusieve® agarose gel (FMC Corp., Rockland, Me.). Amplification reactions which yielded amplified products were compared with the known human chromosome complement of the somatic hybrid panel to determine the location of the translocation partner gene.

For the radiation hybrid screen, the primers used were the same as those used for the somatic cell hybrid screen. The 20 microliter PCR reaction mixtures each comprised 25 nanograms of the DNA to be screened from the Stanford G3 radiation hybrid panel (Research Genetics, Huntsville, Ala.), 0.5 unit Taq Gold® DNA polymerase (Perkin Elmer Cetus, Norwalk, Conn.), 250 micromolar of each dNTP, PCR reaction buffer at 1×final concentration (Perkin Elmer Cetus, Norwalk, Conn.), and 5 picomoles of each of two primers. After initial denaturation and Taq Gold activation at 95° C. for 10 minutes, a two-phase touchdown protocol for annealing and extension was used. In the first phase, sixteen cycles were performed by maintaining the PCR reaction mixture at 95° C. for 45 seconds, and at 70° C. for 1 minute (decreasing by 0.7° C./cycle) to reach a final combined annealing and extension temperature of 59° C. In the second phase, twenty-six cycles were performed by maintaining the PCR reaction mixture at 95° C. for 45 seconds, at 55° C. for 30 seconds, and at 72° C. for 1 minute. A final elongation was performed at 72° C. for 5 minutes. Aliquots of each PCR reaction mixture were electrophoresed in a 4% (w/v) Nusieve® agarose gel (FMC Corp., Rockland, Me.). PCR amplification reactions which yielding an amplified polynucleotide product and reactions which did not yield an amplified polynucleotide product were scored as 1 and 0, respectively. Results were submitted to the radiation hybrid server of the Stanford Human Genome Center (at World Wide Web address http://www-shgc.stanford.edu/rhserver2/rhserver_form.html) to determine the location of the partner DNA.

The location of the translocation partner gene was further verified by FISH analysis of a subclone derived from a panhandle PCR-amplified polynucleotide product. The probe was labeled with biotin-16-dUITP and FISH analysis was performed on metaphases from peripheral blood lymphocytes obtained from a normal human male using standard methods.

The Partner DNA Originated from Chromosome Band 4q21

To determine the chromosomal location of the partner DNA, we screened panels of somatic cell hybrid DNAs and radiation hybrid DNAs by PCR. Amplification of a PCR product from cell line 803 in the somatic hybrid panel (Bios Laboratories, New Haven, Conn.) indicated that the partner DNA was from human chromosome 4. PCR amplification of radiation hybrid lines in the Stanford G3 radiation hybrid panel demonstrated that the partner DNA was in the same bin as the framework marker D4S1542 at chromosome band 4q21. The PCR primers used to screen the panels of somatic hybrid DNAs and radiation hybrid DNAs were from a more 5′ region of the partner DNA than the 255 bp region of homology to existing sequences of ESTs H73415, G26138 and G29714, as depicted in FIG. 3. Thus, the chromosome band 4q21 location of the ESTs independently corroborated the location of the partner DNA.

For further verification of the location of the partner DNA, a subclone containing the genomic breakpoint junction was used as probe in FISH analysis. The probe consisted of 3651 bp of MLL sequence extending from the nested forward primer to the translocation breakpoint, 3224 bp of sequence from the partner gene, and an additional 75 bp of MLL sequence extending from the ligated phosphorylated oligonucleotide through the reverse nested primer used for PCR. Twenty metaphases from human peripheral blood lymphocytes of a normal male were examined. Signal was detected on at least one chromosome 11 in 9 of 20 cells. Signal was detected at proximal 4q in 5 of 20 cells. Due to the small size of the probe, signal was not detected in every cell. More importantly, however, there was no significant hybridization elsewhere in the genome. These data are consistent with a location of the partner DNA at chromosome band 4q21 and indicate that panhandle PCR amplified a genomic translocation breakpoint involving MLL and partner DNA from chromosome band 4q21.

RT-PCR Analysis.

RT-PCR analysis was performed to evaluate whether translocation fused MLL with AF-4. The Superscript® Preamplification System (Gibco BRL, Gaithersburg, Md.) and random hexamers were used for synthesis of cDNA from 4 micrograms of total RNA obtained from the same infant, according to the manufacturer's directions. The 100 microliter RT-PCR reaction mixtures comprised 2 microliters of a random hexamer-primed cDNA preparation, 2.5 units of AmpliTaq® DNA polymerase (Perkin Elmer Cetus, Norwalk, Conn.), 200 micromolar of each dNTP, PCR reaction buffer at 1×final concentration (Perkin Elmer Cetus, Norwalk, Conn.), and 100 picomoles of each primer. The primers were derived from MLL exon 6 and from the AF-4 gene, and have been described (primers MLLEx6S and LTG4AS2 respectively; Yamamoto et al., 1994, Blood 83:2912-2921). After initial denaturation at 95° C. for 2 minutes, thirty-five cycles were performed by maintaining the PCR reaction mixture at 95° C. for 1 minutes, at 62° C. for 2 minutes, and at 72° C. for 1 minutes. A final elongation was performed at 72° C. for 10 minutes. A second round of RT-PCR was performed using a 2 microliter aliquot of the first RT-PCR reaction mixture as the template. Primers and conditions were the same, except that the annealing temperature was 65° C. The cell line RS4:11, which is known to have an MLL genomic breakpoint within intron 7 and to yield a 627-nucleotide-residue polynucleotide product when amplified using these primers, was the positive control (Yamamoto et al., 1994, Blood 83:2912-2921).

Polynucleotide products amplified by RT-PCR were electrophoresed in VisiGel® Separation Matrix (Stratagene, Inc., La Jolla, Calif.), and aliquots of these products were electrophoresed in 1% (w/v) agarose and purified for nucleotide sequencing using a GENECLEAN III® kit (Bio 101, Inc., La Jolla, Calif.). Seventy nanograms of purified polynucleotide were used for direct automated sequencing using standard methods and the same primers as those used for RT-PCR.

RT-PCR Analysis Indicates MLL-AF-4 Chimeric mRNA

Since the partner DNA originated from chromosome band 4q21, RT-PCR analysis was performed on randomly primed cDNA from the leukemic cells of patient 38 to evaluate whether the translocation joined MLL to AF-4. Initial and second round RT-PCR reactions with sense and antisense primers from MLL and AF-4, respectively, showed the predicted 627 bp product in the positive control cell line RS4:11 (Yamamoto et al., 1994, Blood 83:2912-2921). In the leukemia of patient 38, initial and second round reactions gave a single 741 bp product. Direct sequencing of the products of four separate second round reactions showed an in-frame fusion of MLL exon 8 to the AF-4 gene at position 1459 of the AF-4 cDNA (Nakamura et al., 1993, Proc. Natl. Acad. Sci. USA 90:4631-4635). These data indicate that the unknown partner DNA that panhandle PCR had amplified was from a previously uncharacterized region of the AF-4 gene.

The Translocation Partner Gene is Homologous with EST H73415.

The nucleotide sequence of portions of subclones derived from panhandle PCR-amplified polynucleotide products were identical to known sequences of ESTs H73415, G26138 and G29714. The entire EST H73415 was obtained and sequenced (Genome Systems, St. Louis, Mo.) from the Soares human fetal liver and spleen cDNA library (dbEST Id:375797), in both directions. The EST was 1034 nucleotide residues in length. The EST was homologous with portions of subclones derived from panhandle PCR-amplified polynucleotide products. The homology was in 1033 of 1034 nucleotide residues and extended through an AluJₒ sequence into a region of unique non-repetitive sequence, where the EST subclone ended. Neither the sequence of the amplified portion of the translocation partner gene in the full length products of panhandle PCR, nor the region of homology with EST H73415, contained intron-exon boundaries or shared homology with full-length AF-4 cDNA. These results suggest that the portion of the translocation partner gene which comprised the unknown region of the panhandle PCR-amplified polynucleotide product was derived from a previously uncharacterized intronic region of AF-4.

Automated Sequencing of EST H73415.

EST H73415 (Genome Systems, St. Louis, Mo.), which was derived from the Soares human fetal liver spleen cDNA library (dbEST Id:375797), was obtained as a bacterial stab in the vector pT7T3D-Pac and isolated as individual colonies from a Luria broth agar plate containing 100 micrograms per milliliter ampicillin in the agar. The entire EST was sequenced in both directions using a T3 sequencing primer and sequencing primers used to characterize the translocation partner gene.

Summary of Findings by Panhandle PCR in this Example

The translocation partner DNA comprised unique non-repetitive sequences, Alu and MaLR (mammalian apparent LTR-retrotransposon) repetitive sequences, and a region having homology with known expressed sequence tags (ESTs) H73415, G26138, G29714 of the Human Genome Database. A diagram of the translocation breakpoint region of the MLL gene of the infant patient is shown in FIG. 3.

MaLR sequences have not previously been associated with leukemia-associated translocation breakpoints. The non-repetitive sequences were not homologous to any known partner gene of MLL. Screening of somatic cell hybrid and radiation hybrid lines by PCR and fluorescent in situ hybridization (FISH) analyses of normal metaphase chromosomes mapped the translocation partner DNA to chromosome band 4q21. Reverse transcriptase PCR (RT-PCR) identified an MLL-AF-4 chimeric mRNA, which indicated that a fusion of MLL with a previously uncharacterized intronic region of AF-4 had occurred. This Example of basic panhandle PCR amplification of a MLL genomic breakpoint region demonstrated that the method is useful for identifying an unknown translocation partner gene of MLL.

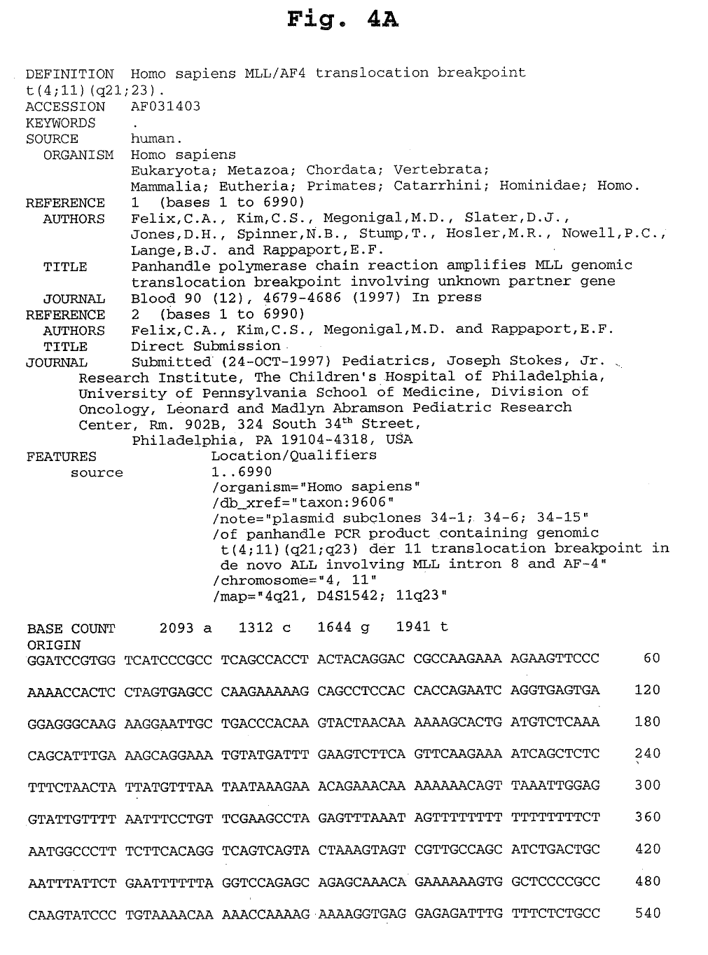

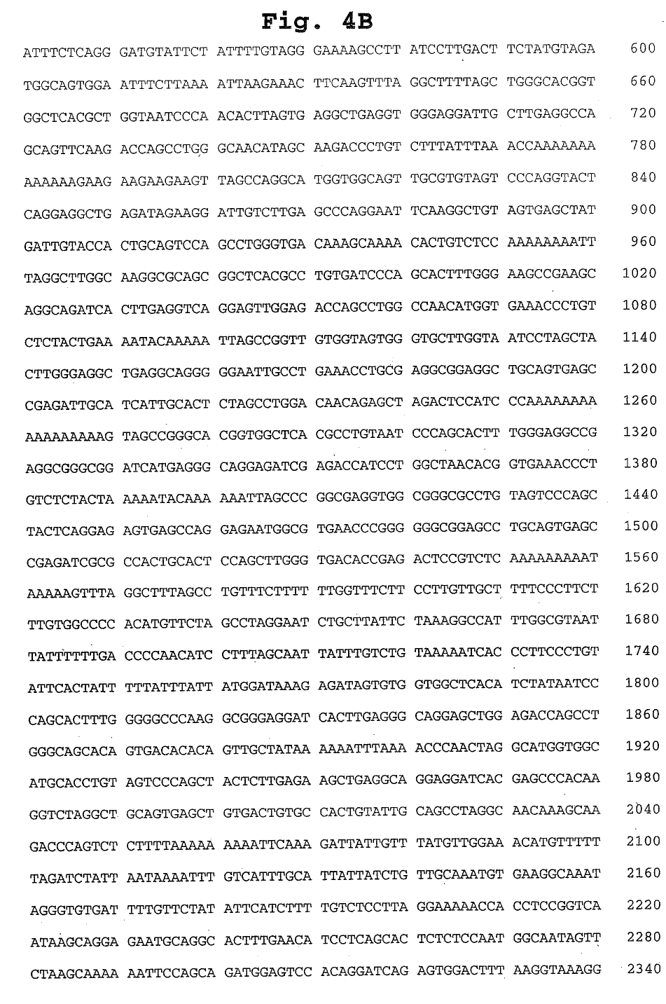

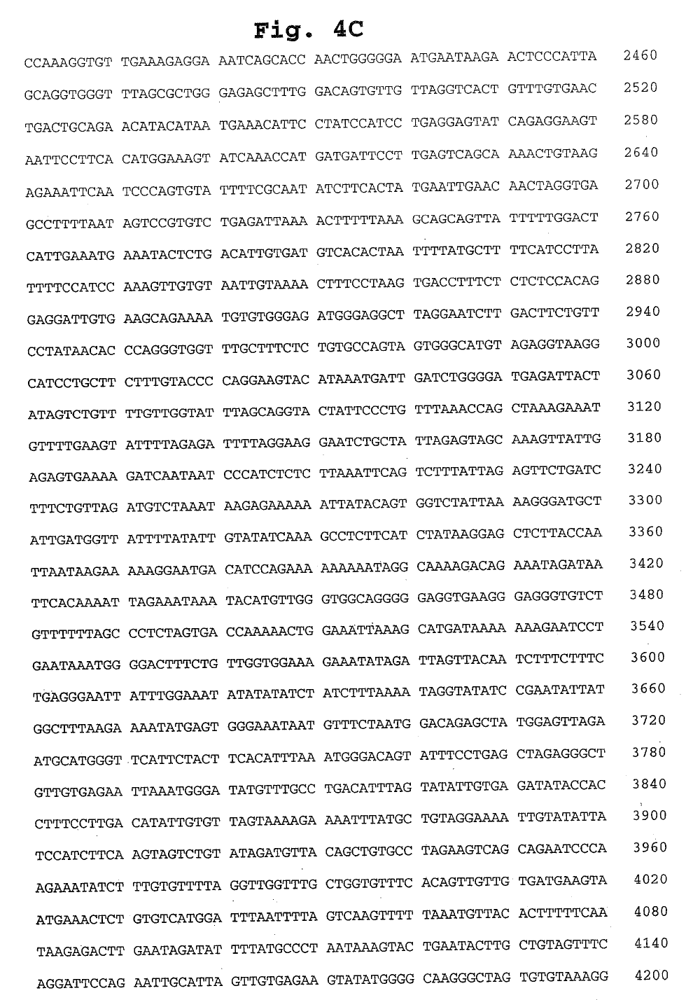

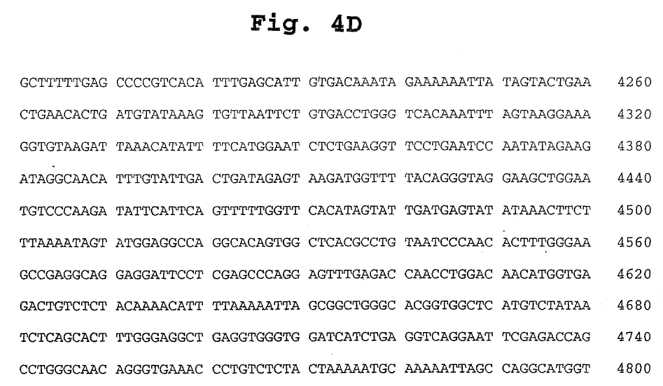

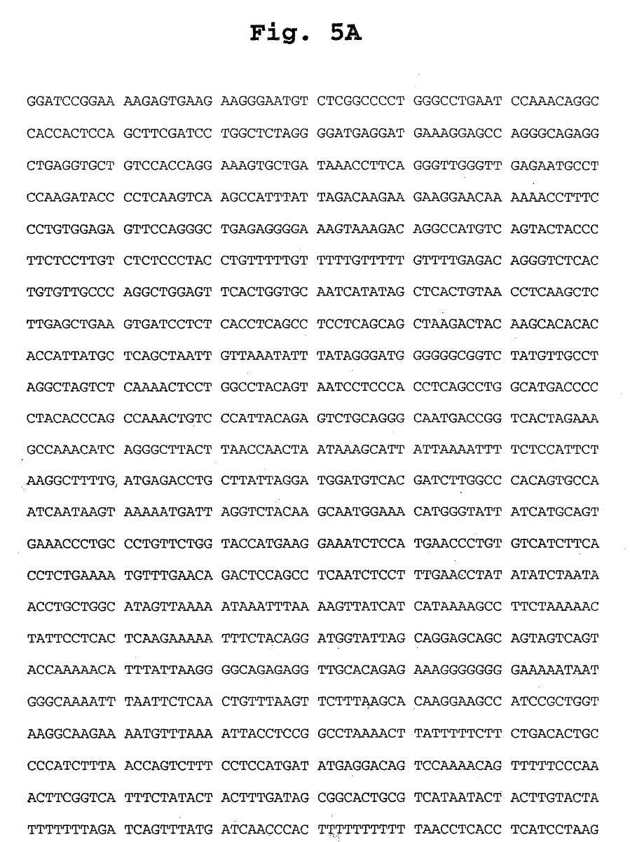

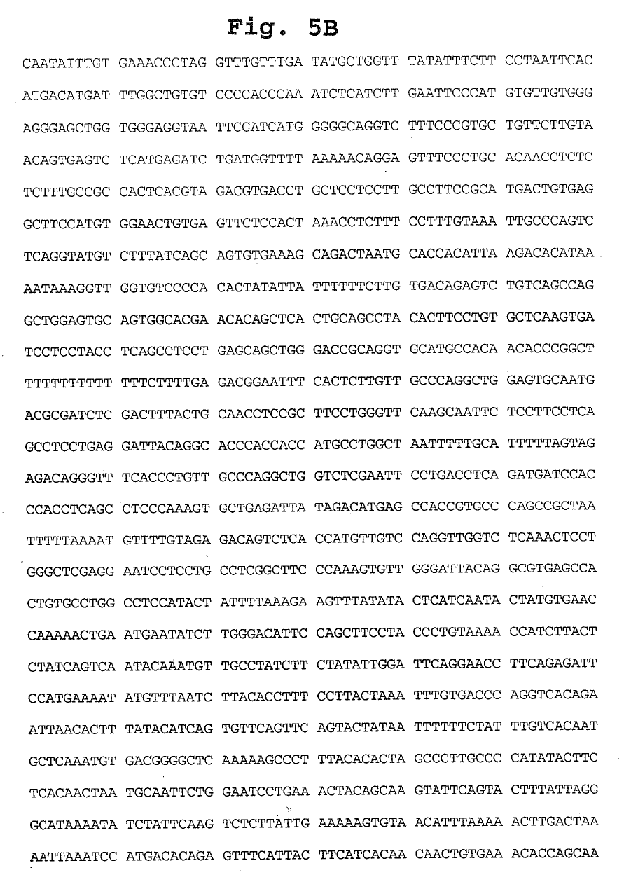

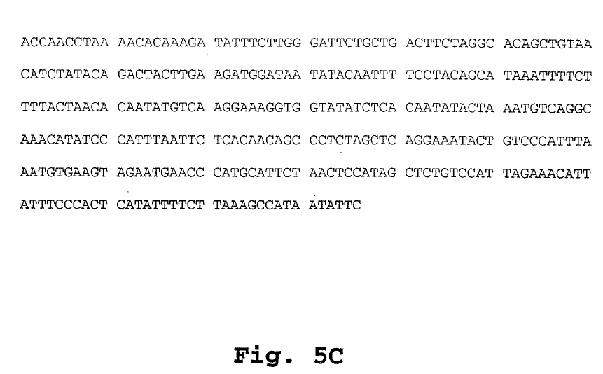

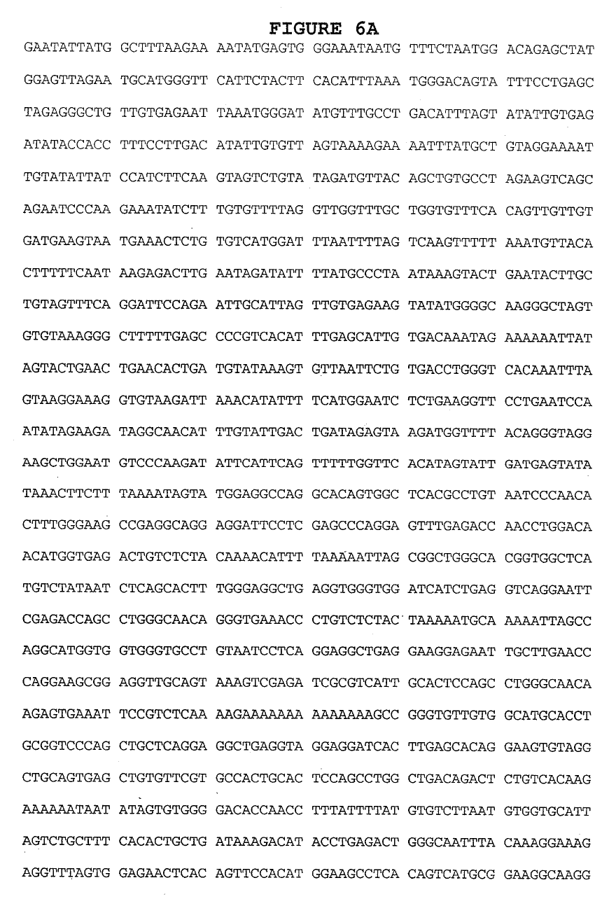

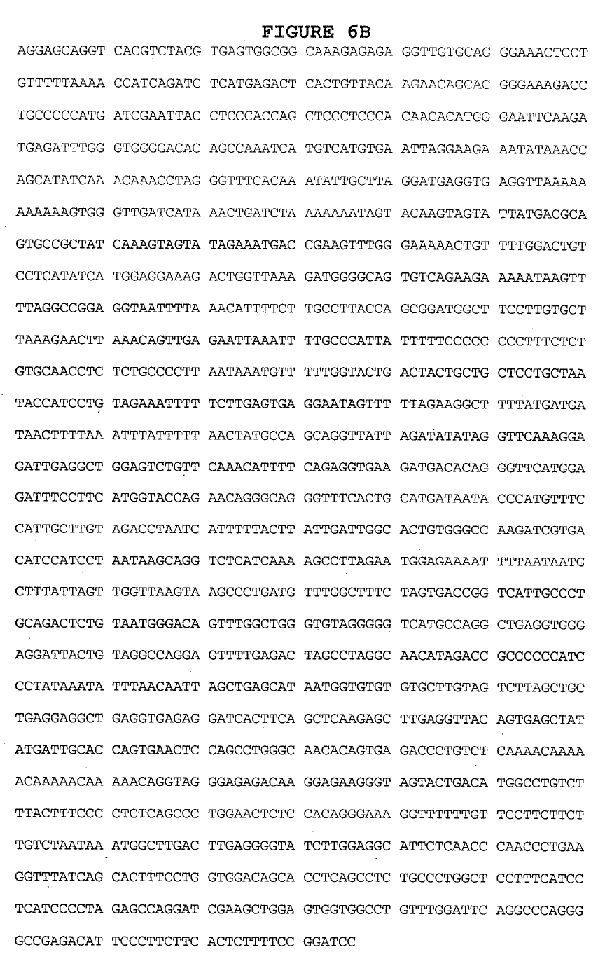

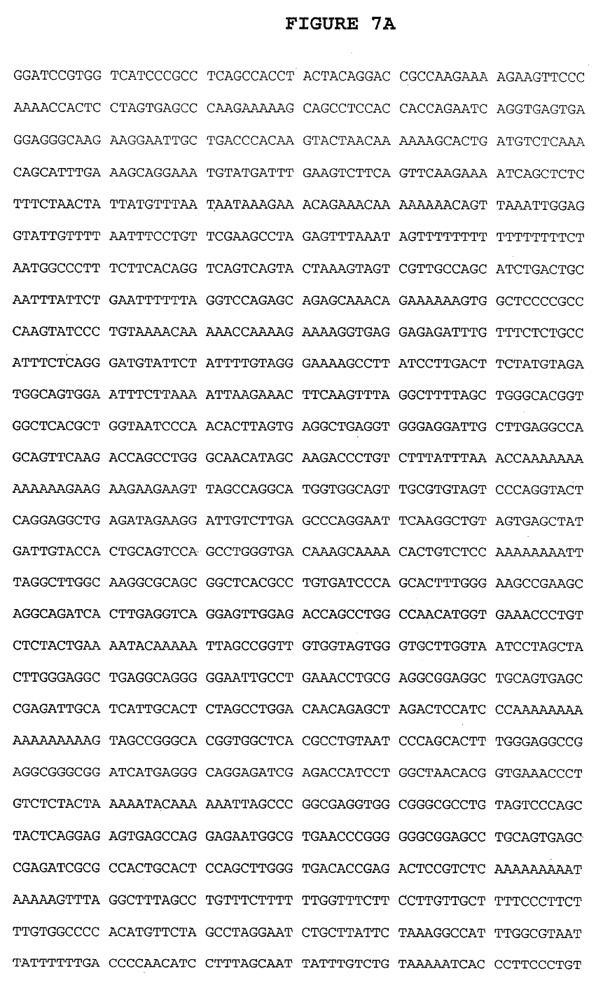

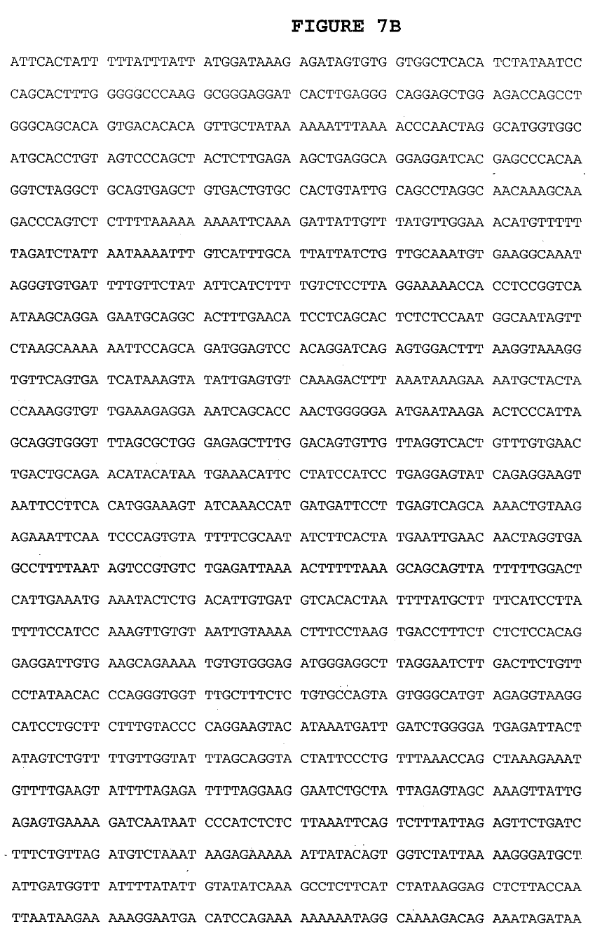

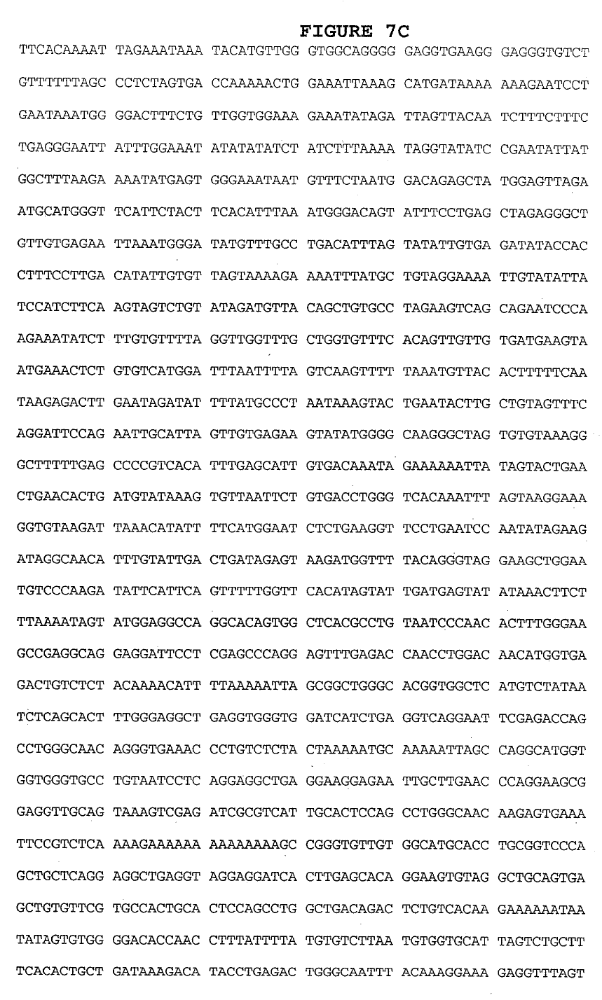

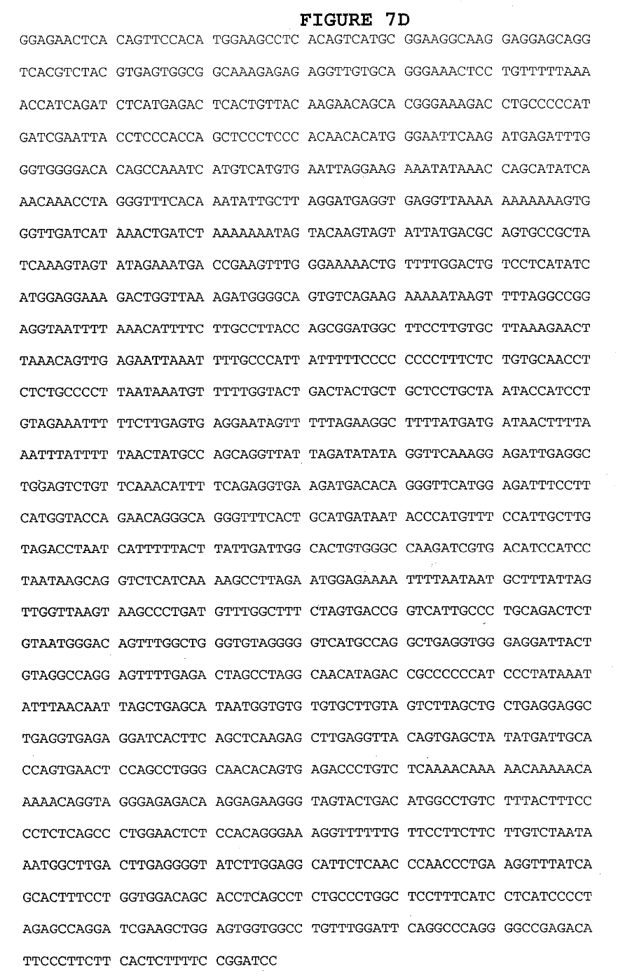

The nucleotide sequence of a portion of the gene sequence obtained from the unknown region of the amplified polynucleotide product derived from the infant patient described in this Example is listed in FIG. 5 (SEQ ID NO: 1). The antisense sequence corresponding to this portion of the gene sequence is listed in FIG. 6 (SEQ ID NO: 2). A conglomerate nucleotide sequence derived from numerous subclones of the amplified polynucleotide product derived from the infant patient described in this Example is listed in FIG. 7 (SEQ ID NO: 3). This conglomerate nucleotide sequence begins at the 5′-BamHI site, and extends through the MLL derived sequence to the 3′-BamHI site. A subsequently corrected sequence was deposited with GenBank (Accession Number AF031403), and is listed in FIG. 4 (SEQ ID NO: 23).

### Examples 2 and 3

Panhandle PCR Amplifies MLL Genomic Breakpoints in Treatment-related Leukemias

Panhandle PCR amplifies genomic DNA with known 5′ and unknown 3′ sequences. We used panhandle PCR to clone MLL genomic breakpoints in one case each of treatment-related acute lymphoblastic leukemia (t-ALL) (Example 2) and treatment-related acute myeloid leukemia (t-AML) (Example 3). By adding sequence to the unknown 3′ partner DNA that was complementary to a known MLL 5′ sequence and intrastrand annealing, we were able to generate the genomic template with an intrastrand loop for panhandle PCR. The methodology was exactly as describe above as in Example 1 for MLL genomic breakpoint cloning in the case of infant ALL, except that the amount of Taq/Pwo used was 1.75 units and 5 min rather than 7 min was used for annealing/elongation in the PCR reactions because the target sequences were smaller.

### Example 2

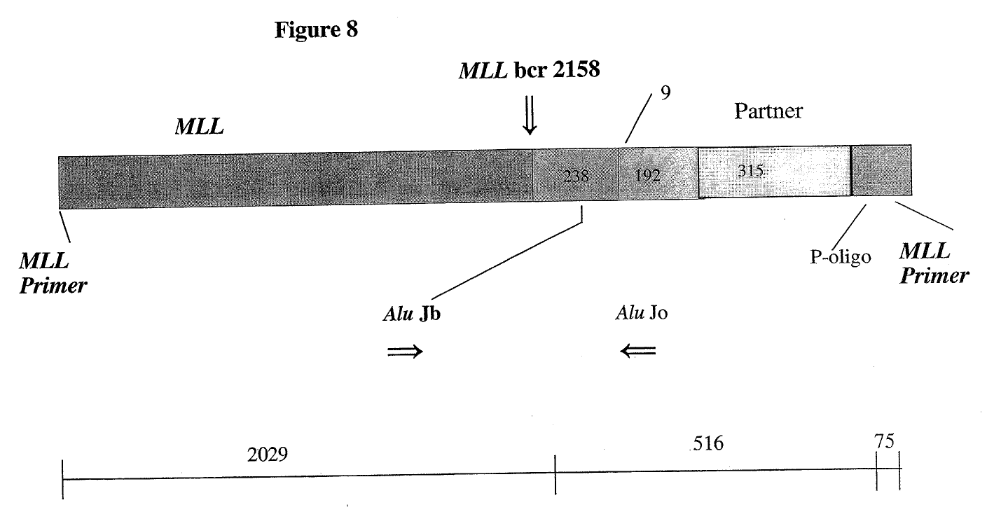

Southern blot analysis of the ALL of patient 33 with the t(4;11)(q21;q23) revealed 7 kb and 2.5 kb rearrangements. Panhandle PCR products 2620 bp in size indicated that the 2.5 kb restriction fragment on Southern blot analysis was from the der(11) chromosome. Automated sequencing of three subclones of these products identified the MLL genomic breakpoint at nucleotide 2158 in intron 6 in the 5′ bcr, as depicted in FIG. 8. For further confirmation of the breakpoint sequence, genomic DNA from the leukemic cells was amplified with a primer set encompassing the translocation breakpoint. Direct sequencing verified that nucleotide 2158 was the breakpoint in the MLL bcr.

Sequences of the breakpoint and the partner DNA 3′ of the breakpoint were the same in all three subclones. The breakpoint in MLL was within an Alu element of the J subfamily. Five hundred sixteen bp of sequence 3′ of the breakpoint represented partner DNA, followed by sequences of the ligated oligonucleotide and the reverse primer used for nested PCR. The partner DNA also contained an AluJ that began 9 bp downstream fromtranslocation breakpoint. The more 3′ sequence of the partner DNA was rich in short poly-A and poly-T repeats.

Consistent with the karyotype, screening of the Stanford G3 radiation hybrid panel with PCR primers from the partner DNA indicated that the nearest linked marker to the partner DNA was D4S1542 at chromosome band 4q21. These results validated the panhandle PCR method in a treatment-related leukemia where the cytogenetic location of the partner DNA was known.

Although the leukemia showed a t(4;11)(q21;q23) translocation, the non-repetitive partner DNA sequences did not share homology with known genomic sequences of AF-4. However, screening of radiation hybrid panel DNAs previously indicated that the nearest linked marker to the AF-4 gene was also D4S1542, suggesting that the partner DNA in the treatment-related ALL was derived from either AF-4 or from a genomic sequence in close proximity to AF-4.

We performed RTPCR analysis as described for the case of infant ALL above. RT-PCR analysis showed that the t(4;11) was an MLL-AF-4 fusion, indicating that the partner DNA in the products of panhandle PCR was another previously uncharacterized AF-4 intronic sequence.

In summary, the karyotype in the t-ALL showed t(4;11)(q21;q23). Panhandle PCR amplified the translocation breakpoint at position 2158 in intron 6 in the 5′ MLL genomic breakpoint cluster region (bcr). The sequence of the partner DNA was not homologous to cDNA or genomic sequences of the AF-4 gene at chromosome band 4q21, the most common partner gene of MLL in ALL. Nonetheless, RT-PCR analysis showed that the t(4;11) was an MLL-AF-4 fusion, indicating that the partner DNA in the products of panhandle PCR was another previously uncharacterized AF-4 intronic sequence.

### Example 3

Panhandle PCR Identifies MLL Partial Duplication in Treatment-related AML with 46. XY Karyotype

In the AML of patient 13 where the karyotype was 46, XY, Southern blot analysis revealed a single 3.5 kb rearrangement in BamHI digested DNA. Panhandle PCR products 3446 bp in size were obtained, consistent with the single rearrangement on Southern blot analysis. Sequencing of a subclone identified the breakpoint at position 1493 in intron 6 of the MLL bcr. PCR amplification of genomic DNA from the leukemic cells with a primer set encompassing the breakpoint and direct genomic sequencing confirmed that nucleotide 1493 was the rearrangement breakpoint in the MLL bcr.

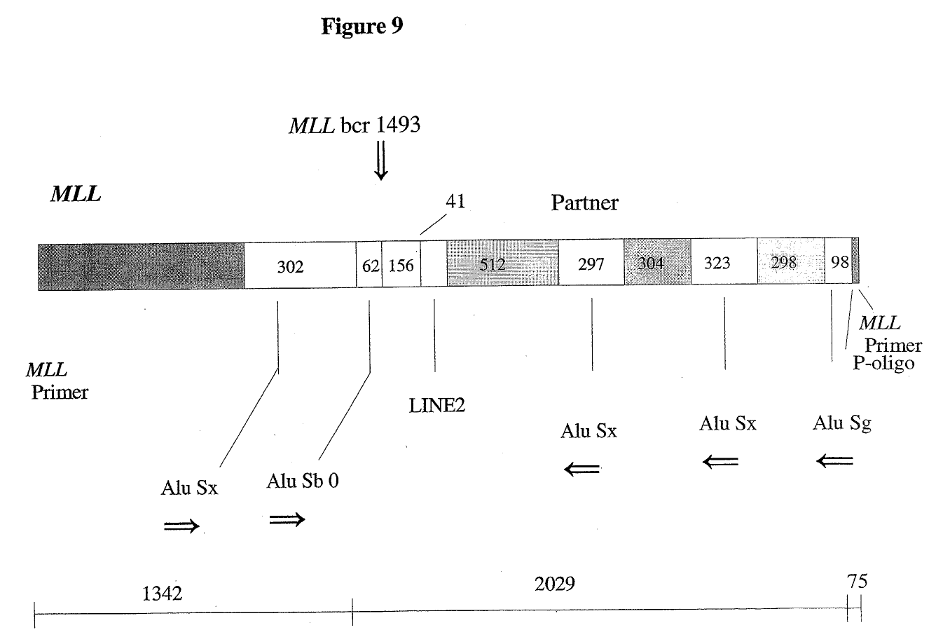

The breakpoint in MLL intron 6 was within an AluS repeat, as depicted in FIG. 9. Two thousand twenty-nine bp of sequence 3′ of the breakpoint were from partner DNA, followed by sequences of the ligated oligonucleotide and the reverse primer used for nested PCR. The partner DNA contained four unique sequence regions, one LINE2 and three additional AluS repeats. The breakpoint in the partner DNA was in a unique sequence region.

Single rearrangements on Southern blot analysis sometimes indicate partial duplication of several exons of the MLL gene (Schichman et al., 1994, Proc. Natl. Acad. Sci. USA 91:6236-6239; Nakao et al., 1996, Leukemia 10:1911-1918). However, the partner DNA did not share homology with known genomic sequences of MLL and the small size of the rearranged BamHI fragment was not consistent with other partial duplications analyzed with BamHI (Schichman et al., 1994, Proc. Natl. Acad. Sci. USA 91:6236-6239; Caligiuri et al., 1996, Cancer Res. 56:1418-1425; So et al., 1997, Cancer Res. 57:117-122; Yamamoto et al., 1997, Am. J. Hematol. 55:41-45). To determine the chromosomal location of the partner DNA, panels of somatic cell hybrid DNAs and radiation hybrid DNAs were screened by PCR. Amplification of a PCR product from cell line 1049 in the somatic cell hybrid panel (Bios Laboratories) indicated that the partner DNA was on human chromosome 11. PCR amplification of radiation hybrid lines in the Stanford G3 radiation hybrid panel showed that the partner sequence was in the same bin as the framework marker D11S2060 at chromosome band 11q23.3.

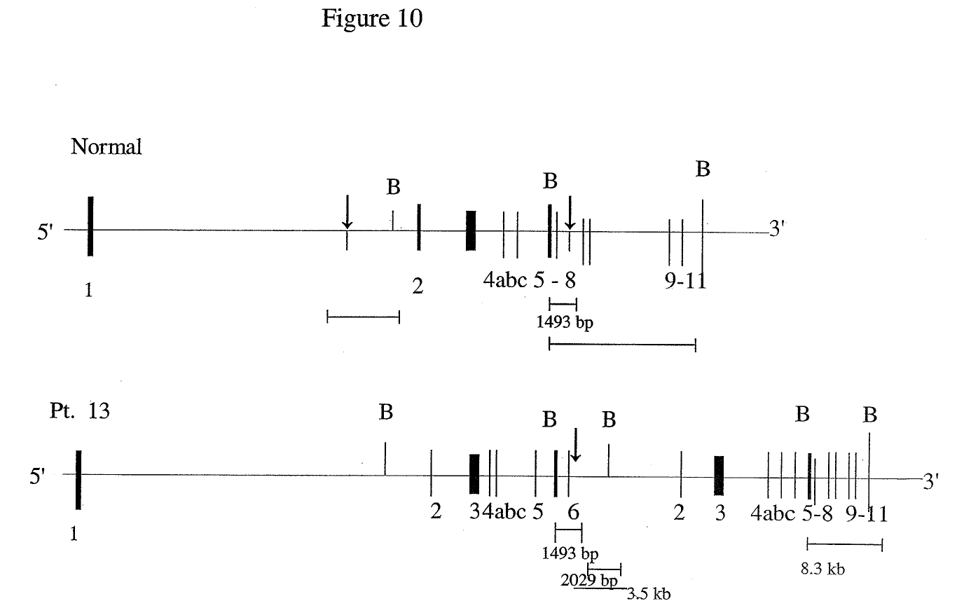

RT-PCR analysis was performed on total RNA from the leukemic cells to evaluate whether the fusion was an MLL partial duplication. Nested RT-PCR reactions with sense and antisense primers from MLL exon 6 and exon 3, respectively, gave a single 228 bp product. Direct sequencing of the products of three separate nested RT-PCR reactions revealed an in-frame fusion of exon 6 to exon 2, indicating that panhandle PCR identified an MLL partial duplication that joined intron 6 with intron 1, 2029 bp upstream of the intron 1 BamHI restriction site, as indicated in FIG. 10.

In summary, in Example 3 the karyotype in the t-AML was normal, but Southern blot analysis showed a single MLL gene rearrangement. Panhandle PCR amplified the breakpoint at position 1493 in MLL intron 6, also in the 5′ bcr. Screening of somatic cell hybrid and radiation hybrid DNAs by PCR and RT-PCR analysis of the leukemic cells indicated that panhandle PCR identified a fusion of MLL intron 6 with a previously uncharacterized sequence in MLL intron 1, consistent with a partial duplication.

In both the t-ALL of Example 2 and the t-AML of Example 3, the breakpoints in the MLL bcr were in Alu repeats and there were Alu repeats near the breakpoints in the partner DNAs, suggesting that the repetitive sequences were important for these rearrangements. Analysis of additional pediatric cases will determine whether breakpoint distribution deviates from the predilection for 3′ distribution in the bcr that has been found in adult cases. These results show that panhandle PCR is an effective method for cloning MLL genomic breakpoints in treatment-related leukemias. Panhandle PCR may be the prototypic PCR approach for identification of translocation breakpoints where the 3′ sequence of the partner gene is undetermined and materials are limited. In all 3 cases described so far, the sequence of the partner DNA was previously uncharacterized intronic sequence of a known partner gene of MLL, showing the power of this method to characterize new genomic sequences flanking translocation breakpoints.

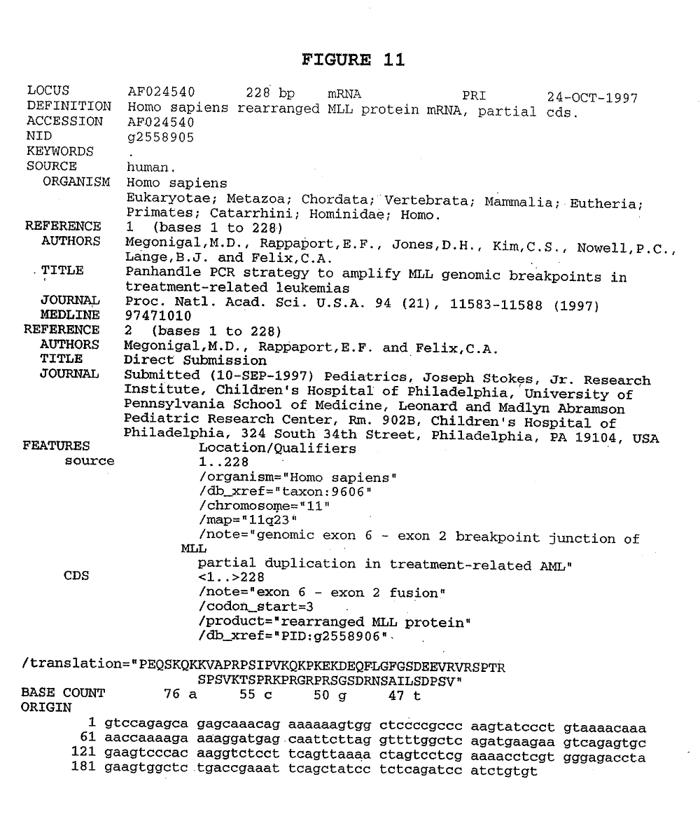

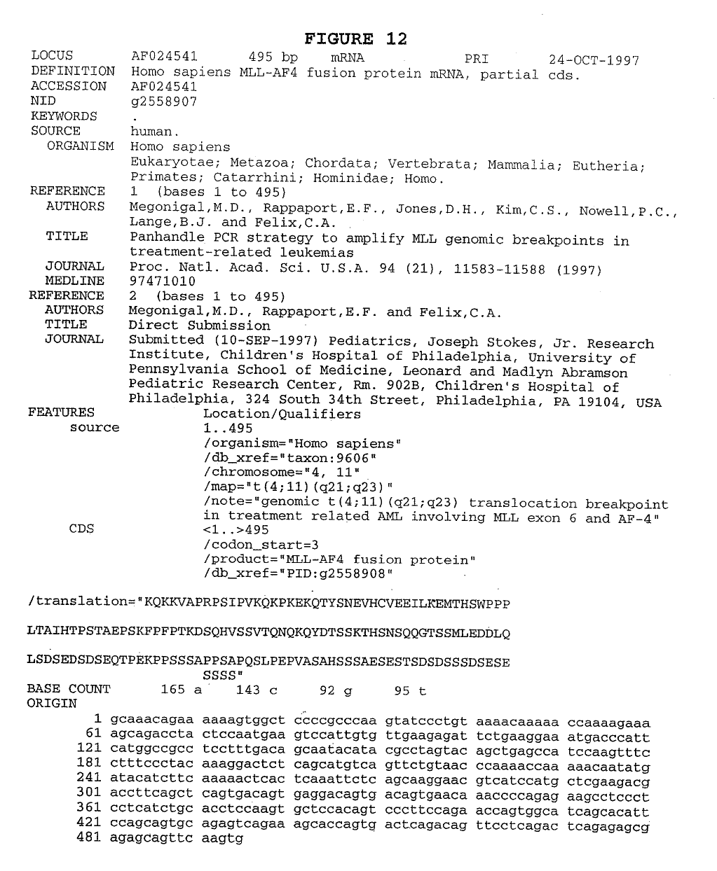

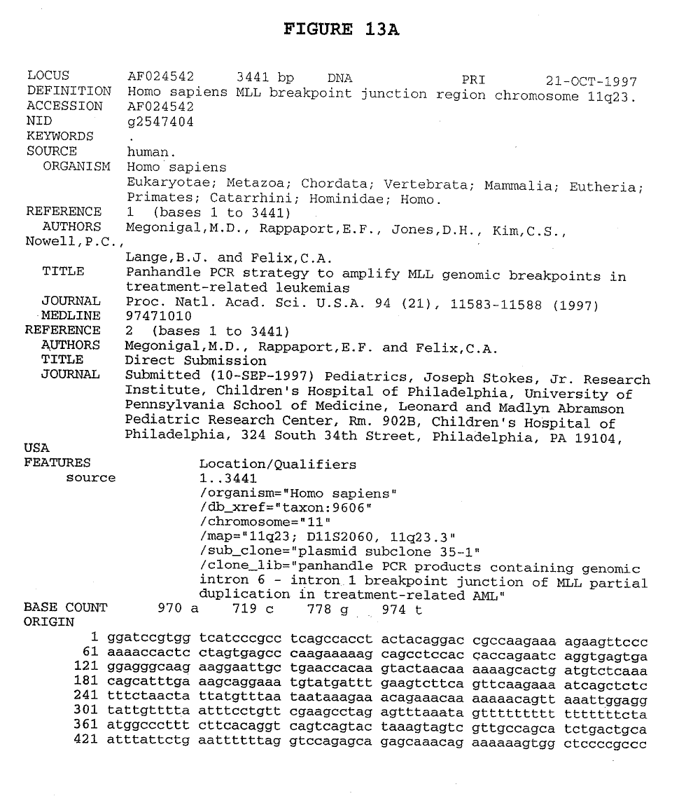

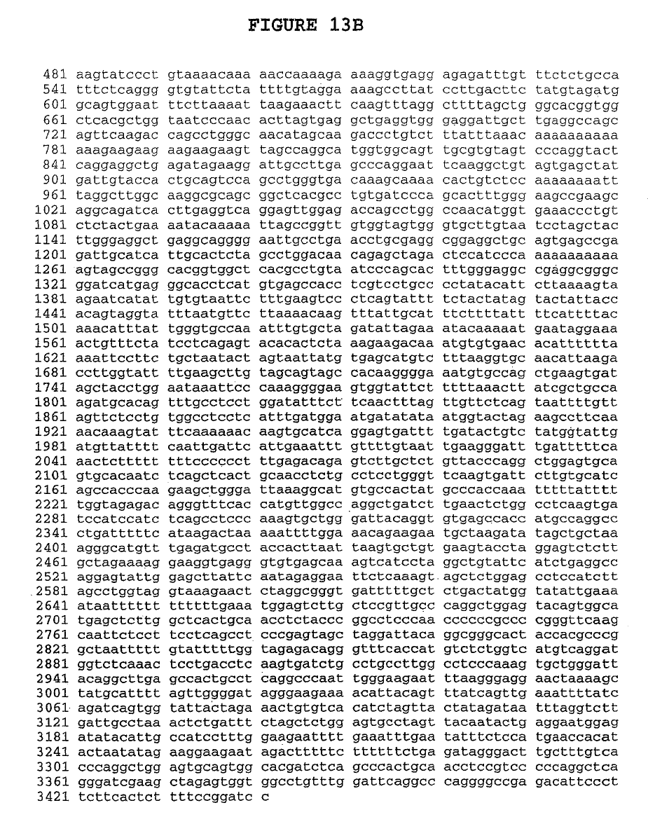

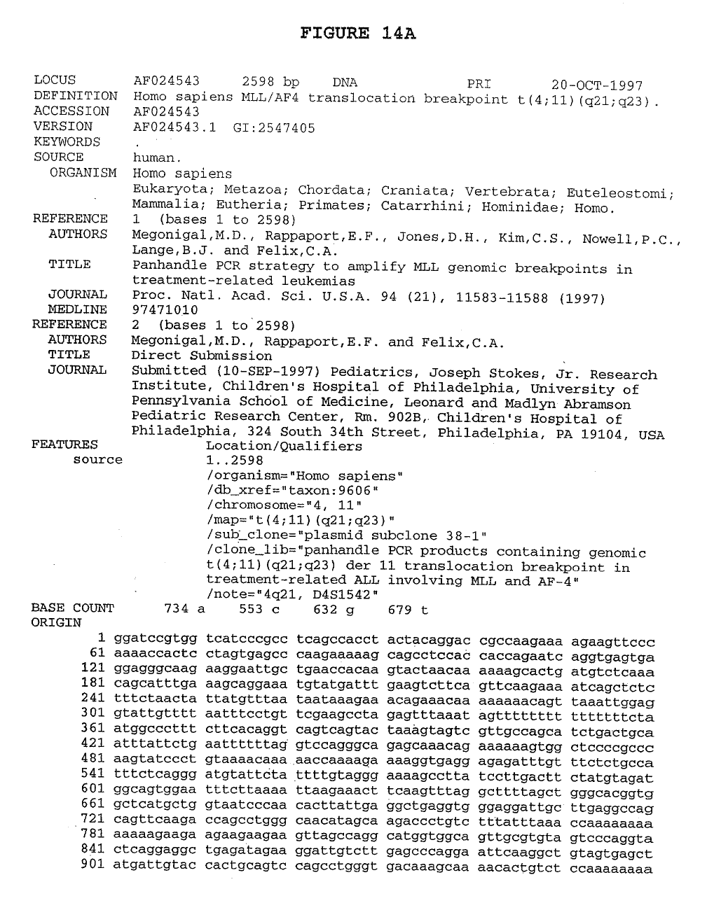

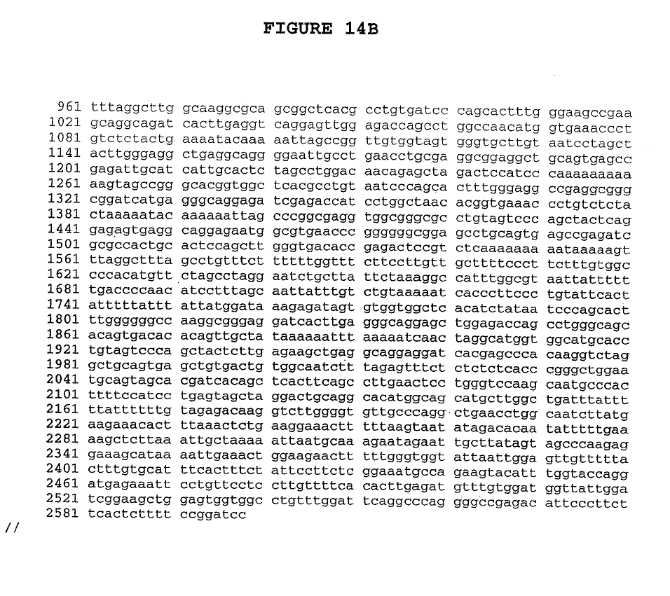

The GenBank accession numbers for sequences in examples 2 and 3 are AF024540-AF024543 and the sequence are shown in FIGS. 11-14 (SEQ ID NOs: 15, 16, 17, and 18).

### Example 4

Recombination PCR Simplifies Cloning of MLL Genomic Breakpoint Regions by Panhandle PCR

Conventional subcloning methods involve ligations of the ends of panhandle PCR products with ends of linearized plasmid, followed by transformation of E. coli. Subcloning of panhandle PCR products was simplified by using recombination PCR in place of conventional Subcloning methods. Recombination PCR has been described (Jones et al., 1991, BioTechniques 10:62-66).

To subclone panhandle PCR products by recombination PCR, a PCR reaction was performed using a HindIII-digested plasmid template (pUC19) to generate a linearized plasmid polynucleotide having ends complementary to the ends of the panhandle PCR-amplified polynucleotide products. PCR products from both PCR reactions are combined, undergo in vivo recombination after transformation of E. coli. Recombinant plasmid, which comprised a panhandle PCR-amplified polynucleotide product, were identified by PCR, rather than by preparing and digesting minipreps, as in conventional methods.

### Example 5

Recombination PCR was used in conjunction with panhandle PCR to characterize the breakpoint regions in two infant patients afflicted with ALL, each of whom exhibited t(4;11). In both of these patients, similar MLL rearrangements were identified, the rearrangements being 9.5 kilobases and 3.2 kilobases in size. Based on these observations, it appeared that the translocation breakpoint regions of the MLL genes of the two patients might be similar. In one of the two patients, panhandle PCR amplification generated a polynucleotide product 3.2 kilobases in length. When recombination PCR was used for retrieval and detection of the MLL genomic breakpoint region of this patient, ten of seventeen subclones generated in three separate panhandle PCR reactions were observed to comprise the desired 3.2 kilobase polynucleotide product. Nucleotide sequencing of the subclones identified the genomic breakpoint at position 1737 of the bcr of MLL, in MLL intron 6, at a position 21 nucleotides 3′ with respect to an AluSbO repeat sequence. The breakpoint in the translocation partner DNA of this patient was located 3 nucleotides 5′ with respect to a LINE2 repeat. Furthermore, GG, TTT, AG and TG nucleotide sequences were present on both sides of the breakpoint junction, suggesting that base pairing and homologous end-joining are involved in the translocation process.

Panhandle PCR amplification of DNA obtained from leukemic cells of the other patient generated a polynucleotide product 3.2 kilobases in length. When recombination PCR was used for retrieval and detection of the MLL genomic breakpoint region, seventeen of twenty-four subclones comprised the 3.2 kilobase insert. Nucleotide sequencing of the subclones identified the genomic breakpoint at position 914 of the bcr of MLL, in MLL intron 6 and within an AluJ repeat. An AluJ_{b} repeat was located 32 nucleotides 3′ with respect to the breakpoint in the partner gene. As in the other patient, GGG, CT, TT, and AA nucleotide sequences were present at both sides of the breakpoint junction. Although the breakpoint regions in the translocation partner DNAs of these two patients were different, overlapping nucleotide sequences in the subclones placed them in the same intronic region. The sizes of the rearrangements detected by genomic Southern blot analysis of these two patients and the locations of BamHI sites in AF-4 were consistent with the presence of genomic breakpoints in AF-4 intron 3 in both cases.

### Example 6

Panhandle Variant PCR Amplified an MLL Genomic Breakpoint Region in a Patient Afflicted with Treatment-related MDS Involving an Unknown Partner Gene

The variant panhandle PCR described herein was used to clone the MLL genomic breakpoint region of a patient afflicted with treatment-related myelodysplastic syndrome (MDS). The patient was diagnosed at 13 years 9 months of age with a monocytic preleukemia, 11 months after the start of treatment for primary neuroblastoma. The patient's neuroblastoma treatment involved administration of DNA topoisomerase II inhibitors, alkylating agents, and radiation. The patient's marrow karyotype indicated a del(11q23) during the period of preleukemia, which lasted for six months before onset of overt FAB M4 AML. Although the karyotype indicated del(11q23), detection of two MLL gene rearrangements by Southern blot analysis indicated that translocation had occurred.

The variant panhandle PCR method described in this Example was used to amplify the unknown translocation partner DNA sequence which was present at the breakpoint region of the der(11) chromosome. This method amplified a polynucleotide product having a length of 6.0 kilobases. Recombination PCR was performed to retrieve and detect the MLL genomic breakpoint region. Nucleotide sequencing of the subclones identified the genomic breakpoint at position 4664 of the bcr of MLL, in MLL intron 8. The nucleotide sequence of the translocation partner DNA was not homologous to any known partner gene of MLL, suggesting that the translocation partner DNA was either a previously uncharacterized intronic region of a known translocation partner gene of MLL or a novel translocation partner gene. Screening of somatic cell hybrid and radiation hybrid lines by PCR was performed to determine the chromosomal location of the translocation partner DNA. This suggested that the partner DNA was from chromosome 17. Thirty-four of the sixty-eight subclones from four variant panhandle PCR reactions comprised the desired insert, which was 6.0 kilobases in size. These results demonstrate the usefulness of the variant panhandle PCR method for cloning a translocation breakpoint region comprising a portion of an unknown partner gene. In addition, it shows that a long product was obtained by this technique.

### Example 7

t(11;22)(q23;q11.2) in Acute Myeloid Leukemia of Infant Twins Fuses MLL with hCDCrel, a Cell Division Cycle Gene in the Common Region of Deletion in DiGeorge and Velocardiofacial Syndromes

Case Histories

Patient 68 presented at 11½ months of age with fever, bruising, thrombocytopenia, WBC of 228×10⁹/liter and leukemia in the central nervous system. The bone marrow was replaced by blasts of French-American-British (FAB) M2 morphology that expressed CD33 and CD45. The G-banded karyotype was 46,XX,t(11;22)(q23;q11.2)[15], while fluorescence in situ hybridization (FISH) analysis with an MLL-specific probe (Oncor) showed hybridization with the normal chromosome 11 and split signals on the der(11) chromosome and chromosome 22. The patient was a monozygous twin. Seven weeks later, the twin of patient 68, designated patient 72, was also diagnosed with AML. Patient 72 presented with bruising and WBC of 20.6×10⁹/l. There were 67% abnormal blasts of FAB M1 morphology on marrow differential. The blasts expressed HLA-DR, CD13 and CD33. The G-banded karyotype of the diagnostic marrow was 46,XX[5]/46,XX,t(11;22)(q23;q11)[15]. On fluorescence in situ hybridization analysis, the MLL-specific probe hybridized with the normal and der(11) chromosomes and chromosome 22, suggesting that the t(11;22)(q23;q11.2) disrupted MLL.

Southern Blot Analysis Identifies Identical MLL Gene Rearrangements in Infant Twins

We examined peripheral blood mononuclear cells from patient 68 and leukemic marrow cells from patient 72 for MLL gene rearrangement at times of diagnosis. In both cases, the B859 probe showed the 8.3 kb germline band and identical, rearranged BamHI restriction fragments 3.8 kb and 6.3 kb in size, indicating chromosomal translocation. Pre-diagnosis peripheral blood mononuclear cells were obtained from patient 72 at time of diagnosis of leukemia in her twin. On two-week exposure, Southern blot analysis of the pre-diagnosis specimen detected both the germline band and faint 3.8 kb and 6.3 kb rearrangements, showing presence of cells with the translocation before clinical leukemia appeared. The intensity of the rearrangements relative to the germline band increased from pre-diagnosis to the time of diagnosis.

Panhandle PCR Variant Amplifies MLL Genomic Translocation Breakpoint

We first implemented panhandle variant PCR to clone the MLL genomic breakpoint on the der(11) chromosome in the leukemia of patient 68. Six independent panhandle PCR variant reactions yielded products ˜3.9 kb in size, indicating that the 3.8 kb rearrangement on Southern blot analysis was from the der(11) chromosome. There was sufficient material for direct genomic sequencing of the translocation breakpoint junction without subcloning the products of panhandle variant PCR. In addition, to confirm the translocation breakpoint and obtain additional information on the partner DNA, we performed recombination PCR using the products of one panhandle variant PCR reaction. Six of eight recombination PCR-generated subclones contained the desired 3.9 kb insert and we sequenced two subclones in their entirety.

The t(11;22)(q23;q11.2) Fuses MLL with hCDCrel, a Cell Division Cycle Gene in the Common Region of Deletion in DiGeorge and Velocardiofacial Syndromes

Direct automated sequencing of the products of panhandle variant PCR identified the genomic breakpoint at nucleotide 2672 in MLL intron 7 and provided partial sequence of the partner DNA. Sequencing of the subcloned products confirmed the translocation breakpoint and yielded additional sequence of the partner gene. A BLAST search against the nucleotide database indicated that the sequence of the partner DNA at chromosome band 22q11.2 was identical to an intronic region of the hCDCrel (human cell division cycle related) gene (Accession No. 000093), which is a member of a gene family involved in cell division cycle that includes the Drosophila peanut-like protein 1 gene. The hCDCrel gene maps to the central portion of a 1.3 Mb sequence contig on chromosome band 22q11.2 that is commonly deleted in DiGeorge and velocardiofacial syndromes.

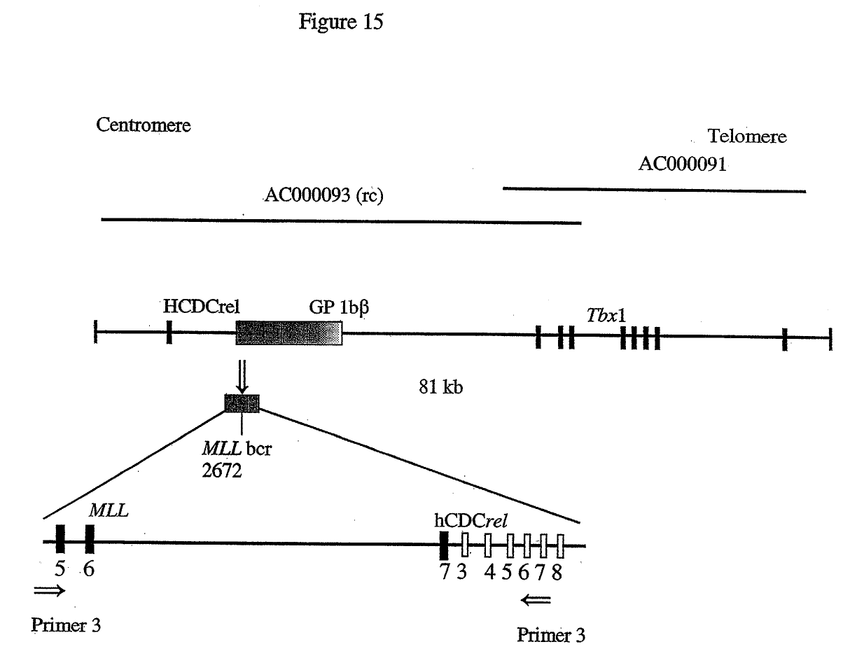

Comparison of the cDNA to the genomic sequence indicates that hCDCrel contains 11 exons that span approximately 9 kb. The genomic breakpoint in hCDCrel in the leukemia of patient 68 was in intron 2 at nucleotide 26510 relative to cosmid carlaa (Accession No. 000093), although the orientation of the GenBank entry (Accession No. 000093) is in opposite orientation to the open reading frame of the cDNA. Consistent with the size of the rearrangement on Southern blot analysis, and with the size of the panhandle variant PCR product, the next BamHI site in the hCDCrel gene 3′ of the translocation breakpoint is located in exon 8 at position 25275 of cosmid carlaa (Accession No. 000093). Thus, 2622 bp of sequence in the panhandle variant PCR product were from MLL and 1240 bp were from hCDCrel. FIG. 15 depicts the translocation breakpoint junction region of the der(11) chromosome in relation to the chromosome band 22q11.2 genomic region. The region of the genomic breakpoint in hCDCrel was rich in simple repeats and low complexity repeats. Both MLL and hCDCrel contained homologous CT, TTTGTG and GAA sequences within a few base pairs of their respective breakpoints.

Independent Confirmation of MLL Genomic Breakpoint in Leukemia of Patient 68

To detect the translocation breakpoint by a method that was independent of panhandle variant PCR, we amplified fresh aliquots of genomic DNA from the leukemic cells of patient 68 with primers encompassing the translocation breakpoint, which were designed from sequences of the products of panhandle variant PCR. Four independent PCR reactions gave the predicted 344 bp product. Direct sequencing was performed on the products of two reactions and verified the translocation breakpoint.

RT-PCR Analysis Shows MLL-hCDCrel Chimeric mRNA

Since the partner DNA originated from hCDCrel at chromosome band 22q11.2, we performed RT-PCR analysis on randomly primed cDNA from the leukemic cells of patient 68 to evaluate whether the translocation produced a fusion mRNA. The RT-PCR reaction performed with sense and antisense primers from MLL exon 6 and hCDCrel exon 3, respectively, gave the predicted 247 bp product. Direct sequencing of the products of RT-PCR showed an in-frame fusion of MLL exon 7 to hCDCrel exon 3 at position 142 of the 2032 bp full-length hCDCrel cDNA (Accession No.U74628).

Panhandle Variant PCR Amplifies Identical MLL Genomic Translocation Breakpoint in AML of Patient 72

We also used panhandle variant PCR to isolate the translocation breakpoint junction in the AMLof patient 72, the twin of patient 68. The products of one panhandle variant PCR reaction were subcloned by recombination PCR. The desired 3.9 kb insert was present in 6 of 7 subclones and two positive subclones were sequenced in entirety. The sequence showed the same MLL intron 7 breakpoint at nucleotide 2672 and the same hCDCrel partner DNA as in the leukemia of patient 68 . For independent confirmation, we amplified fresh aliquots of genomic DNA from the leukemic cells of patient 72 with primers encompassing the breakpoint junction. Four independent PCR reactions gave the predicted 344 bp product. We directly sequenced the products of two of the reactions, which verified the translocation breakpoint.

Summary and Significance of Findings in AML of Infant Twins

Using panhandle variant PCR technology, we determined that the t(11;22)(q23;q11.2) in concordant AMLs of monozygous infant twins was the result of fusion of MLL with hCDCrel and identified a new partner gene of MLL at chromosome band 22q11.2. The panhandle variant PCR results were validated independently by direct genomic sequencing of products of conventional PCR and by RT-PCR analysis. The genomic sequence of the partner DNA at the translocation breakpoint junction of the der(11) chromosome was identical to intron 2 of the hCDCrel gene at chromosome band 22q11.2 (Accession No. 000093). hCDCrel is a member of a gene family involved in cell division cycle that includes the Drosophila peanut-like protein 1 gene. The hCDCrel gene contains 11 exons that span approximately 9 kb and yields two transcripts of ˜2.5 and ˜3.5 kb (16). The smaller transcript terminates at an imperfect polyadenylation site, while the longer transcript is produced by the alternative use of the polyadenylation site of Glycoprotein (GP) Ibβ, the adjacent 3′ gene. The putative protein product of hCDCrel is a GTP-binding protein.

The hCDCrel gene is in the central portion of a 1.3 Mb sequence contig, which is part of the region on chromosome band 22q11.2 commonly deleted in both DiGeorge and velocardiofacial syndromes. DiGeorge syndrome is a constitutional disorder characterized by cardiac anomalies, thymic and parathyroid hypoplasia and dysmorphic craniofacial features, while the major features of velocardiofacial syndrome are palatal and cardiac defects, facial dysmorphia and learning disabilities. hCDCrel is the second partner gene of MLL located in a region of the genome involved in both leukemia and a constitutional disorder. In 1996, Borrow et al. determined that the t(8;16)(p11;p13) of AML represents a fusion of the MOZ and CBP (CREB-binding protein) genes. Shortly afterwards, Taki et al. and Sobulo et al. demonstrated that CBP is the partner gene of MLL in myelodysplastic syndrome with the t(11;16)(q23;p13.3). CBP encodes a histone acetyltransferase that functions as a transcriptional coactivator. The Rubinstein-Taybi syndrome, a constitutional disorder that includes mental retardation, dysmorphic facial features, and broad thumbs and toes, is characterized by chromosomal translocations, microdeletions and point mutations of the CBP gene. Thus, there is some precedent for involvement of the same region of the genome in both developmental abnormalities as well as in leukemia.

We detected short homologous sequences two to six bp in length at the breakpoint junctions in both MLL and hCDCrel. Similarly, we found short segments of homology between MLL and AF-4 or AF-9 at t(4;11) and t(9;11) breakpoint junctions. On the basis of these findings, we proposed that base pairing of homologous DNA ends of MLL and partner gene is one step in the translocation process. In addition, the cloning of a constitutional balanced t(2;22)(q14;q11.21) translocation associated with DiGeorge syndrome identified several small segments of nucleotides (˜6 bp) repeated on chromosomes 2 and 22, suggesting that the same phenomenon may occur in constitutional and somatic translocations.

The t(11;22)(q23;q11.2) that fused MLL with hCDCrel in the leukemias of infant twins is distinct from the constitutional t(11;22)(q23;q11) translocation, which is the most frequent, recurrent, non-Robertsonian translocation in humans. In the constitutional t(11;22), the phenotype is normal and the translocation is present in all cells. Furthermore, the breakpoints at chromosome band 11q23 in the constitutional recurrent translocations map proximal to cancer-associated translocation breakpoints involving MLL. Two lines of evidence argue that the t(11;22)(q23;q11.2) that we observed was not constitutional. In patient 72 where serial samples were available, the intensity of MLL gene rearrangements relative to the germline band on Southern blot analysis progressively increased from pre-diagnosis to the time of diagnosis. Furthermore, the karyotype of the diagnostic marrow of patient 72 revealed 5 of 20 cells in which the karyotype was normal.

Concordance of the unique, clonal, non-constitutional MLL gene rearrangements s suggests that the t(11;22) occurred in utero and that there was metastasis from one twin to the other via the placenta. The ages of the two twins at diagnosis of leukemia were similar, 11.5 months and 13 months. Within pairs of twins, the ages at onset of leukemia have generally been concordant, suggesting similar times of latency before disease is evident. The delineation of MLL gene rearrangements in twins as in utero events complements research efforts on prenatal exposures to environmental toxins as etiologic factors in leukemia in infants. One line of investigation involves maternal dietary DNA topoisomerase II inhibitors, since leukemias in infants resemble treatment-related leukemias linked to chemotherapy that targets DNA topoisomerase II. Moreover, the latency to onset of disease suggests a potential role for secondary alterations in addition to the translocations, but the influence of various translocation partners on sufficiency of MLL gene translocations for full leukemogenesis has not been addressed.

Using panhandle variant PCR, we amplified a 3.9 kb product, identified the t(11;22) translocation breakpoint and distinguished hCDCrel as a new partner gene of MLL in AML of infant twins. The method was devised to simplify the PCR-based cloning of genomic DNAs with unknown 3′ flanking sequences, precisely the situation with many MLL genomic breakpoints. Beyond the finding of a new partner gene of MLL, this work introduces a particular PCR technology that expedites translocation breakpoint cloning. We recently used the original panhandle PCR as another strategy to clone MLL genomic breakpoints. Although the names are similar because the genomic template in both cases has an intrastrand loop schematically shaped like a pan with a handle, panhandle variant PCR is distinct from the original panhandle PCR. Both strategies offer advantages over conventional genomic cloning and conventional long-range PCR, which requires specific primers for the many partner genes of MLL. Increased use of both methods will test whether one or the other is more advantageous in specific situations. Furthermore, for retrieval and detection of the MLL genomic breakpoint, we employed recombination PCR, which uses E. coli itself to mediate DNA recombination and obviates the ligation step in subcloning.

Including the hCDCrel gene identified in the present work, 13 partner genes of MLL have been cloned to date. In addition, MLL may fuse with self in partial tandem duplications. The joining of the MLL breakpoint cluster region and several different partner genes renders molecular cloning of MLL genomic breakpoints by PCR more difficult. Panhandle variant PCR offers a new strategy to surmount the challenge of cloning MLL genomic breakpoints where the partner genes are many and often undetermined. Identification of a genomic region at chromosome band 22q11.2 involved in AML and in the constitutional DiGeorge and velocardiofacial syndromes also is of interest.

### Example 8

Panhandle PCR Strategy to Amplify Genomic Breakpoints in Human Disorders

Maintenance of the structural integrity of chromosomes is a critical cellular function. Substantial changes in the normal arrangement of nucleic acid sequences (i.e., genes) on chromosomes frequently results in serious abnormalities. Rearrangements within or between chromosomes, for example, may produce embryos that are developmentally impaired and/or not viable. Chromosome translocations, wherein segments of two different chromosomes are exchanged, provide an example of one type of rearrangement that occurs in the human genome. Some translocations, particularly those that do not alter the total amount of genetic material, are physiologically benign. Many chromosome translocations, however, have been linked to human disorders. Indeed, the presence of particular translocations can provide a diagnostic indicator regarding the predisposition of an individual for a particular disease, which in turn provides information regarding the necessity of screening relatives of an affected individual. To date, however, the information and technology required to render such analyses are lacking and the task of identifying and characterizing many disease-linked translocations has yet to be performed.

Cancer is perhaps the most prevalent of the human disorders associated with chromosomal translocations. As described above, human acute leukemias, for example, have been linked to a variety of chromosomal translocations. The genomic breakpoint junction sequences of both derivative chromosomes have been examined in relatively few de novo and treatment-related leukemias that represent the spectrum of the many known and unknown partner genes of MLL. The large number of potential partner genes can impede genomic cloning. Panhandle PCR approaches were used successfully to clone the der(11) genomic breakpoint junctions as described in the previous examples (Megonigal et al., 2000, Proc Natl Acad Sci, USA 97: 2814-2819; Felix et al., 1997, Blood 90: 4679-4686; Felix et al., 1998, Leukemia 12: 976-981; Megonigal et al., 1997, Proc Natl Acad Sci USA 94: 11583-11588).

Many cancers have been linked to translocations between chromosomes that generate oncogenes or fusion genes at the chromosomal translocation junction. Such oncogenes are frequently comprised, in part, of genes encoding transcription or developmental regulators, which are master regulators of cell fate (e.g., LMO2 which is involved in acute leukemia). Other translocations linked to cancer etiology include those in which a translocation juxtaposes the regulatory elements of one gene proximal to the coding region of a second gene, thereby altering the normal expression pattern of the second gene. Jumping translocations (JTs) and segmental jumping translocations (SJTs), on the other hand, are unbalanced translocations involving a donor chromosome arm or chromosome segment that has fused to multiple recipient chromosomes. In leukemia, where JTs have been predominantly observed, the donor segment (usually 1q) preferentially fuses to the telomere regions of recipient chromosomes. JT breakpoints of both donor and recipient chromosomes have been found to coincide with numerous fragile sites and common integration sites for human DNA viruses. Common fragile sites are thought to be “hot spots” for translocations, as well as deletions, in certain cancers. (Fang et al., 2001, Genes Chromosomes Cancer 30:292-298). The JTs within each tumor cell line promote clonal expansion, perhaps due to the acquisition of extra copies of donated chromosome segments that frequently comprise oncogenes (i.e., Myc, Abl, Her2/neu), which confer a selective growth advantage. Clonal expansion may lead to genomic imbalances that are tumor-specific (Padilla-Nash et al., 2001, Genes Chromosomes Cancer 30:349-363)

Notably, particular types of chromosomal translocations are usually associated with specific cell types and correlate with particular cancers. The correlation between a particular chromosomal translocation and a specific type of cancer provides means to assess the predisposition of an individual for a cancer and provides a diagnostic tool with which to determine onset of disease and/or disease progression. Analysis of the type of chromosomal translocation is also of utility for accurate diagnosis of cancer subtypes that have similar clinical presentation, but diverge with respect to their prognosis and therefore require different therapeutic regimes to optimize patient survival. Different subtypes of alveolar rhabdomyosarcoma (ARMS), for example, possess translocations characteristic of the subtype. The more common translocation observed in ARMS fuses the PAX3 and FKHR genes and patients with PAX3-FKHR-positive ARMS exhibit reduced event-free survival rates as compared to patients with the ARMS subtype characterized by the less common translocation that fuses the PAX7 and FKHR genes. A recent study has determined that PAX3-FKHR-positive ARMS tumor cells exhibit a greater degree of cell cycle dysregulation than that observed in PAX7-FKHR-positive ARMS tumor cells (Collins et al., 2001, Med Pediatr Oncol 37:83-89). The differential cellular responses observed for the above ARMS subtypes provides useful information for designing optimal therapeutic regimes for treatment of patients having either PAX3-FKHR-positive or PAX7-FKHR-positive ARMS. Such studies underscore the need to examine chromosome translocations on a molecular level in order to render an accurate diagnosis of a patient and provide efficacious treatment.

The present invention provides methods for defining and delineating a specific chromosomal translocation on a molecular level and, therefore, provides a clinician with the information required to diagnose a patient accurately and treat such a patient following an optimized therapeutic regimen. Moreover, the methods of the present invention also provide a sensitive and accurate protocol to monitor a patient's response to a course of therapeutic treatment and detect the presence of minimal residual disease.

The methods of the present invention also provide means to evaluate the predisposition of a patient to a disorder or disease known to be associated with a chromosomal translocation. Thus, the present invention provides means to regularly screen a patient prior to onset of disease to determine if such a patient is a candidate for prophylactic treatment. Such prophylactic treatment may involve, for example, therapeutic intervention with drugs and/or lifestyle changes that would reduce the likelihood of disease onset.

The methods of the present invention are superior to those of other available techniques directed to the analysis of chromosomal translocations because they are exquisitely sensitive and enable the molecular characterization of translocations in which there are large duplications, deletions, inversions or complex rearrangements, or in which the der(11) sequence or the partner gene is unknown. Moreover, the methods of the present invention were used to identify a new partner gene of MLL in a chromosomal rearrangement that involved a cryptic, complex translocation.

The following materials and protocols enable the practice of the methods of Example 8.

Methods and Materials

Case histories. Patient 45 was diagnosed with ALL at age 3 weeks. She presented with massive hepatosplenomegaly and a WBC count of 86×10⁹/L, but no evidence of CNS involvement. The bone marrow karyotype in five metaphases was 46,XX,t(4;11). The immunophenotype was Tdt+, CD19+, CD10−, CD20−; no myeloid antigens were expressed. At age 5 months she had onset of a progressive seizure disorder with loss of milestones, but head MRI and CT scans were normal. By age 10 months, myeloblasts in the cerebrospinal fluid (CSF) suggested central nervous system (CNS) relapse with lineage shift. She suffered rapid neurologic deterioration and died within days.

Patient t-120 was diagnosed with stage IV neuroblastoma at age 2 years. The primary tumor of the posterior mediastinum was locally invasive and there was metastatic disease in the bone and marrow. Memorial Sloan Kettering N7 treatment included four cycles of cyclophosphamide, doxorubicin and vincristine (CAV), three cycles cisplatin and etoposide (PVP), surgical resection, local radiation, radiolabeled anti-G_{D2} monoclonal antibody (3F8), and autologous bone marrow rescue with cells harvested after chemotherapy cycle 5 (PVP), and purged ex vivo with 3F8. Eleven months after starting treatment and two weeks after transplant, the patient was asymptomatic but the WBC count was 46×10⁹/L and FAB L2 ALL was diagnosed. The karyotype in 17 metaphases was 46,XY,t(4;11)(q21;q23). The presentation of patient 38 with infant ALL diagnosis has been described (Felix et al., 1997, Blood 90: 4679-4686; Felix et al., 1998, J Pediatr Hemato/Oncol. 20: 299-308). See Example 1. The 3 month-old girl presented with massive hepatosplenomegaly and a WBC count of 399×10⁹/L. The marrow was replaced with FAB L1 Tdt+, CD19+, CD10−, CD20−, CD34+ lymphoblasts. Cytogenetic analysis of the diagnostic marrow was unsuccessful (Felix et al., 1997, Blood 90: 4679-4686). She received intensive CCG1883-like chemotherapy (Reaman et al., 1999, J Clin Oncol. 17: 445-455) but relapsed in the marrow at 4 years of age, 25 months from completion of this treatment. The marrow karyotype at relapse was 47,XX,t(4;11)(q21;q23),del(7) (q21q31),+8 in three metaphase cells examined. She died from Pseudomonas sepsis one month later during reinduction.

Detection of MLL Gene Rearrangements.

The 8.3-kb MLL bcr was examined by Southern blot analysis of BamHI digested DNA using the B859 cDNA fragment of ALL-1 exons 5-11 (Felix et al., 1998, J Pediatr Hematol/Oncol. 20: 299-308; Felix et al., 1995, Blood 85: 3250-3256).

Characterization of der(11) Genomic Breakpoint Junctions.

For the leukemia of patient 38, characterization of the MLL-AF-4 genomic breakpoint junction by panhandle PCR has been described (GenBank accession number AF031403) (Felix et al., 1997, Blood 90: 4679-4686). See Example 1. For the leukemias of patients 45 and t-120, the der(11) genomic breakpoint junctions were amplified by panhandle PCR after generation of the stem-loop templates from 2.5 μg of genomic DNA as described (Felix et al., 1997, Blood 90: 4679-4686; Felix et al., 1998, Leukemia 12: 976-981; Megonigal et al., 1997, Proc Natl Acad Sci USA 94: 11583-11588), except that primers 3 and 4 used for nested PCR did not contain BamHI sites for ligation. All primers were sense with respect to MLL exon 5. The sequences of primers 3 and 4 were 5′-GGA AAA GAG TGA AGA AGG GAA TGT CTC GG-3′ (SEQ ID NO: 52) and 5′-GTG GTC ATC CCG CCT CAG CCA C-3′ (SEQ ID NO: 24); these primers have been used before for cDNA panhandle PCR (Megonigal et al., 2000, Proc Natl Acad Sci, USA 97: 2814-2819; Megonigal et al., 2000, Proc Natl Acad Sci USA 97: 9597-9602). The panhandle PCR products were subcloned by recombination PCR (Megonigal et al., 2000, Proc Natl Acad Sci, USA 97: 2814-2819; Megonigal et al., 1998, Proc Natl Acad Sci USA 95: 6413-6418). pUC19 was linearized by HindIII digestion and MLL ends complementary to the ends of the panhandle PCR products to be inserted were added to the vector during PCR using primers 5′-ACA TTC CCT TCT TCA CTC TTT TCC TGG CGT AAT CAT GGT CAT AGC-3′ (SEQ ID NO: 25) and 5′-GTG GCT GAG GCG GGA TGA CCA CCA TGC CTG CAG GTC GAC TC-3′ (SEQ ID NO: 26) (Megonigal et al., 2000, Proc Natl Acad Sci, USA 97: 2814-2819). The PCR-amplified pUC19 and panhandle PCR products were purified using GENECLEAN III reagents (Bio 101, La Jolla, Calif.), mixed and added to 50 μl of MAX efficiency DH5α cells (Life Technologies, Gaithersburg, Md.) to recombine in vivo (Megonigal et al., 2000, Proc Natl Acad Sci, USA 97: 2814-2819; Megonigal et al., 1998, Proc Natl Acad Sci USA 95: 6413-6418). Subclones containing panhandle PCR products were identified based on size after PCR with primers 3 and 4 and analyzed by automated sequencing.

Sequences were confirmed by amplification of genomic DNAs with MLL- and AF-4-specific primers followed by direct sequencing. For the leukemia of patient 45, the gene-specific primers were 5′-TGG AAA GGA CAA ACC AGA CC-3′ (SEQ ID NO: 27) from MLL intron 8 (GenBank accession number U04737) and 5′-GTC CCT TAC ATC TGG CAG GA-3′ (SEQ ID NO: 28) from AF-4 intron 3 (GenBank accession number AJ238093). For the leukemia of patient t-120, the gene-specific primers were 5′-CCC ACC CCA CTC CTT TAT ATT-3′ (SEQ ID NO: 29) from MLL intron 8 and 5′-GGC TGC TGG TTT ACA GCT TC-3′ (SEQ ID NO: 30) from AF-4 intron 3.

Cloning of Genomic Breakpoint Junctions of Other Derivative Chromosomes of MLL Translocations by Reverse Panhandle PCR.

Reverse panhandle PCR was accomplished by ligation of a phosphorylated oligonucleotide containing known sense sequence from MLL intron 10/exon 11, which is 3′ in the bcr, to the 3′ ends of BamHI-digested DNA and formation of a stem-loop template from the anti-sense strand. The template, schematically shaped like a pan with a handle, contained unknown partner sequence, the breakpoint junction of the other derivative chromosome, and MLL sequence in the loop. MLL sequence and its complement at either end of the ‘handle’ enabled amplification of the breakpoint junction in three sequential single-primer, two-sided PCRs with primers all anti-sense with respect to MLL exon 11 or intron 10/exon 11 sequences (FIG. 16).

The specific protocol for assessing patients 45 and t-120 is summarized in FIG. 16. In Step 1, 2.5 μg of genomic DNA were digested to completion with 20 units of BamHI (New England Biolabs, Beverly, Mass.). The DNA was treated with 0.025 units of calf intestinal alkaline phosphatase (Boehringer Mannheim, Indianapolis, Ind.) at 37° C. for 30 min and purified using a GENECLEAN III kit (Bio 101). In Step 2, a single stranded 5′ phosphorylated oligonucleotide (5′-GAT CTC TAG ATC TGT ACC AAG TGT GTT CGC TGT AAG AGC-3′: SEQ ID NO: 31) was ligated to the 3′ ends by virtue of the 4-base 5′ end of the oligonucleotide which was complementary to the 5′overhang of the BamHI-digested DNA. The 35-nucleotide 3′ end of the oligonucleotide contained the sense sequence corresponding to positions 8299 to 8333 from MLL intron 10/exon 11 (GenBank accession number U04737). Each 50-μl ligation reaction mixture contained 2.5 μl of DNA, a 50-fold molar excess of the 5′-phosphorylated oligonucleotide, 1 Weiss unit of T4 DNA ligase and 1×ligase buffer (Boehringer Mannheim). Ligations were performed at 4° C. The DNA was purified using a GENECLEAN III kit (Bio 101). The stem-loop template was formed from the antisense strand in Step 3. After heating the other components to 80° C. for 5 minutes, 20 ng of the digested, ligated DNA were added to 1.75 units of Taq/Pwo DNA polymerase mix, 368 μM each dNTP and 1.05×PCR buffer in a 47.5 μl reaction mixture (Expand Long Template PCR System, Boehringer Mannheim). The DNA was rendered single-stranded by heating the reaction mixture at 94° C. for 1 min. The stem-loop structure of the antisense strand (i.e., template) was formed by a 2-min ramp to 72° C. and incubation at 72° C. for 30 seconds (s) to promote intrastrand annealing of the ligated oligonucleotide to the complementary sequence and polymerase extension of the recessed 3′ end (Megonigal et al., 2000, Proc Natl Acad Sci, USA 97: 2814-2819; Megonigal et al., 1998, Proc Natl Acad Sci USA 95: 6413-6418; Felix et al., 1997, Blood 90: 4679-4686; Megonigal et al., 1997, Proc Natl Acad Sci USA 94: 11583-11588; Megonigal et al., 2000, Proc Natl Acad Sci USA 97: 9597-9602; Jones D., 1995, PCR Methods & Applications 4: S195-S201; Pegram et al., 2000, Blood 96: 4360-4362). In Step 4, both primer 1 extension, which makes the template double-stranded, and exponential amplification with primer 1, which anneals to both ends of the template, occur during PCR. 2.5 μl of a 5 pmol/μl solution of primer 1 corresponding to MLL exon 11 antisense positions 8342 to 8315 (5′-GGA TCC ACA GCT CTT ACA GCG AAC ACA C-3′ SEQ ID NO: 44) were added, and each final 50 μl PCR contained 12.5 pmol of primer 1, 350 μM each dNTP and 1×PCR buffer. After initial denaturation at 94° C. for 1 min, 10 cycles at 94° C. for 10 s and 68° C. for 7 min, and 20 cycles at 94° C. for 7 min (increment 20 s/cycle) were utilized, followed by final elongation at 68° C. for 7 min. Steps 5 and 6 include sequential two-sided, single-primer nested PCRs with primers 2 and 3, respectively, which are antisense with respect to MLL exon 11 positions 8336 to 8305 (5′-ACA GCT CTT ACA GCG AAC ACA CTT GGT ACA GA-3′: SEQ ID NO: 32) and MLL intron 10/exon 11 positions 8333 to 8299 (5′-GCT CTT ACA GCG AAC ACA CTT GGT ACA GAT CTA GA-3′: SEQ ID NO: 33). The conditions for the nested PCR amplifications were the same as the initial PCR and 1-μl aliquots of the products of respective preceding PCRs were used as the templates.

Reverse panhandle PCR products were subcloned by recombination PCR (Megonigal et al., 2000, Proc Natl Acad Sci, USA 97: 2814-2819; Megonigal et al., 1998, Proc Natl Acad Sci USA 95: 6413-6418; Megonigal et al., 2000, Proc Natl Acad Sci USA 97: 9597-9602; Jones et al., 1990, Biotechniques 8: 178-183; Jones et al., 1990, Nature 344: 793-794; Jones et al., 1991, Biotechniques 10: 62-66; Jones et al., 1992, Biotechniques 12: 528-535). pUC19 was linearized by HindIII digestion. MLL ends complementary to the ends of the reverse panhandle PCR products generated with primer 3 in the final nested PCR were added to the linearized vector by amplification using primers 5′-ACC AAG TGT GTT CGC TGT AAG AGC TGG CGT AAT CAT GGT CAT AGC-3′ (SEQ ID NO: 34) and 5′ ACC AAG TGT GTT CGC TGT AAG AGC CAT GCC TGC AGG TCG ACT CTA GAG-3′ (SEQ ID NO: 35). The conditions for PCR amplification of the pUC19 and subcloning were as described before (Megonigal et al., 2000, Proc Natl Acad Sci, USA 97: 2814-2819).

The breakpoint junctions of the other derivative chromosomes of the translocations were independently validated by PCR amplification of respective genomic DNAs with gene-specific primers and direct sequencing of the products. For the leukemia of patient 45, the AF-4 intron 3 sense primer 5′-TGT TGG AAA CAA CGG ACA AA-3′ (SEQ ID NO: 36) was used with the MLL intron 8 antisense primer 5′-CAG AGG CCC AGC TGT AGT TC-3′ (SEQ ID NO: 37). For the leukemia of patient t-120, the AF-4 intron 3 sense primer 5′-ATT GTT CTG CCC CCA ACA TA-3′ (SEQ ID NO: 38) was used with the MLL intron 8 antisense primer 5′-TAT TGG ACA TTG CGG GAG AT-3′ (SEQ ID NO: 39). For the leukemia of patient 38, the sense primer 5′-AGA GGC AGG GCA GGA TTT AT-3′ (SEQ ID NO: 40) from CDK6 intron 2 (GenBank accession number AC004128) was used with the MLL intron 9 antisense primer 5′-CTG GAA GAC AGA AAT ACA AAT CAA GA-3′ (SEQ ID NO: 41).

In addition, PCR was performed on genomic DNA from the diagnostic marrow of patient 38 with the sense primer 5′-GAA ATG GGT GCA GTG TTC CA-3′ (SEQ ID NO: 42). from AF-4 intron 3, and the antisense primer 5′-TGG ATT ACG GGA TAG GGA CA-3′ (SEQ ID NO: 43). from CDK6 intron 2, to determine if a reciprocal AF-4-CDK6 rearrangement had occurred, and p53 exon 8 primers were used in a positive control reaction (Felix et al., 1998, Blood 91: 4451-4456).

Reverse Transcriptase-PCR Analysis of Fusion Transcripts.

RT-PCR analysis of the MLL-AF-4 transcript in the marrow of patient 38 at ALL diagnosis (GenBank accession number AF031404) has been described (Felix et al., 1997, Blood 90: 4679-4686). See Example 1. The same approach was used to characterize the der(11) transcript in the ALL of patient 45. First-strand cDNA was synthesized from 1 μg of total RNA with random hexamers using the Superscript Preamplification System (Life Technologies) and amplified with the same sense and antisense primers from MLL exon 6 and AF-4 exon 10 (Felix et al., 1997, Blood 90: 4679-4686). The der(11) transcript in the ALL of patient t-120 was identified by cDNA panhandle PCR using primers, reagents and conditions as described (Megonigal et al., 2000, Proc Natl Acad Sci, USA 97: 2814-2819; Megonigal et al., 2000, Proc Natl Acad Sci USA 97: 9597-9602), and confirmed by amplification of the same first-strand cDNA with the sense and antisense primers 5′-GCA GGC AGT TTG AAC ATC CT-3′ (SEQ ID NO: 45) and 5′-AGG CTT CTC TGG GGT TTG TT-3′ (SEQ ID NO: 46) from MLL exon 7 and AF-4 exons 8/9 (GenBank accession number L13773), respectively.

The der(4) transcripts of the leukemia cells of patients 45 and t-120 were identified by amplification of the first-strand cDNAs (described above) with the AF-4 exon 3 sense primer 5′-CTC CCC TCA AAA AGT GTT GC-3′ (SEQ ID NO: 47; GenBank No. L13773) and the MLL exon 9 antisense primer 5′-CAA TTT TCC AGC TGG TCC TC-3′ (SEQ ID NO: 48; Genbank No. L04284). In the leukemia of patient t-120, the same first-strand cDNA used in cDNA panhandle PCR was amplified with these primers to identify the der(4) transcript.

The CDK-6-MLL transcript was identified in the diagnostic marrow of patient 38 was further characterized with the sense primer 5′-CGT GGT CAG GTT GTT TGA TG-3′ (SEQ ID NO: 49) from CDK6 exons 1-2 (GenBank accession number NM_{—}001259) and the MLL exon 13 antisense primer 5′-GCC GCT CAG TAC AGT TCA CA-3′ (SEQ ID NO: 50) (GenBank accession number L04284). PCR was performed using the same random hexamer-primed cDNA and the AF-4 exon 3 sense primer 5′-CTC CCC TCA AAA AGT GTT GC-3′ (SEQ ID NO: 47) and the CDK-6 exon 4 antisense primer 5′-GAC TTC GGG TGC TCT GTA CC-3′ (SEQ ID NO: 51), to further investigate whether an AF-4-CDK6 rearrangement had occurred.

All cDNAs were amplified with β-actin primers as a positive control (Felix et al., 1997, Blood 90: 4679-4686).

Characterization of der(11) and der(4) Genomic Breakpoint Junctions and Fusion Transcripts from t(4:11) in Infant ALL.

Southern blot analysis of BamHI-digested DNA revealed 6.8 kb and 2.1 kb MLL bcr rearrangements in the infant ALL of patient 45 (FIG. 17A, center). Panhandle PCR identified the der(11) genomic breakpoint junction (FIG. 17A and B). The 6808 bp panhandle PCR products shown in FIG. 17A (left), suggested that the 6.8 kb and 2.1 kb rearrangements on the Southern blot (FIG. 17A, center) were from the der(11) and der(4) chromosomes, respectively. Sequencing of recombination PCR generated subclones revealed the MLL der(11) breakpoint at position 6775 in intron 8 (GenBank accession no. U04737), which is 3′ in the bcr (FIG. 17B). The der(11) breakpoint in the partner gene corresponded to position 34744 in AF-4 intron 3 (GenBank accession no. AJ238093). Sequencing of the 487 bp product of three PCRs performed with forward and reverse MLL and AF-4 clonotypic primers confirmed the der(11) genomic breakpoint junction (data not shown).

AF-4 forward and MLL reverse primers were designed to amplify the der(4) genomic breakpoint junction predicted by the der(11) sequence. The expected product size was 494 bp but a ˜1.2 kb product was obtained (data not shown). The reverse panhandle PCR strategy for genomic cloning of the breakpoint junction of the other derivative chromosome of an MLL translocation was tested in this leukemia with a known der(4) breakpoint junction sequence. The product size of 2232 bp (FIG. 17A, right) was consistent with the 2.1 kb MLL bcr rearrangement from the der(4) chromosome on the Southern blot (FIG. 17A, center), and sequencing revealed the der(4) genomic breakpoint junction (FIG. 17C). The sequence was the same as in the PCR products obtained with AF-4 and MLL-specific primers, validating reverse panhandle PCR as a cloning strategy for the breakpoint junction of the other derivative chromosome of an MLL translocation. The AF-4 der(4) breakpoint mapped to position 34864 or 34865 in intron 3, whereas the MLL der(4) breakpoint mapped to position 6166 or 6167 in intron 8; these breakpoints could not be determined more precisely because both genes contain an adenine ‘A’ nucleotide at the breakpoint junction. Depending on the exact breakpoint positions, 609-610 bases from AF-4 and 121-122 bases from MLL were present in both derivative chromosomes, suggesting that duplication had occurred (FIGS. 17B and C). Additional identical 1-4 base sequences in MLL and AF-4 were present near the der(11) and der(4) breakpoint junctions, suggesting joining of similar DNA ends during this translocation. Relationships of the MLL and AF-4 der(11) and der(4) breakpoints to proximal repetitive sequence elements are shown in FIGS. 17B and 17C. There were AluJo repeats in MLL and AF-4, within ˜1681 bp and ˜259 bp, respectively of the der(11) breakpoint junction. The MLL der(4) breakpoint was within an AluY and there was an AluY in AF-4 intron 3˜988 bp from the der(4) breakpoint junction.

Both der(11) and der(4) transcripts were produced. Three RT-PCRs with MLL- and AF-4-specific primers produced a 742 bp product with an in-frame fusion of MLL exon 8 to AF-4 exon 4 (data not shown). A single RT-PCR with AF-4- and MLL-specific primers gave a 293 base-pair product and sequencing revealed that the der(4) transcript fused AF-4 exon 3 in-frame to MLL exon 9 (data not shown).

Characterization of der(11) and der(4) Genomic Breakpoint Junctions and Fusion Transcripts from t(4;11) in Treatment-related ALL.

Southern blot analysis of BamHI-digested DNA in the treatment-related ALL of patient t-120 revealed 7.2 kb and 2.0 kb MLL bcr rearrangements (FIG. 18A). Panhandle PCR was used to amplify the der(11) genomic breakpoint junction. The presence of a 7295 bp panhandle PCR product suggested that the 7.2 kb and 2.0 kb rearrangements on the Southern blot were from the der(11) and der(4) chromosomes, respectively (FIG. 18A). Sequencing of recombination-PCR generated subclones revealed the MLL der(11) breakpoint at position 6588 or 6589 in intron 8, also 3′ in the bcr, and the der(11) breakpoint in the partner gene at AF-4 intron 3 position 7130 or 7131 (FIG. 18B). The MLL and AF-4 breakpoints could not be localized precisely because both genes contain an ‘A’ nucleotide at the breakpoint juncture. Other 1-4-base homologies were present near the breakpoints in both genes (FIG. 18B). The MLL and AF-4 der(11) breakpoints were near AluJo and other repetitive sequence elements. Sequencing of the 226-bp products of two separate PCRs performed with MLL and AF-4 clonotypic primers confirmed the der(11) genomic breakpoint junction.

The presence of a 2079 bp reverse panhandle PCR product was consistent with the 2.0 kb rearrangement on the Southern blot (FIG. 18A). The AF-4 der(4) breakpoint was position 7108, 7109 or 7110 in intron 3 and the MLL der(4) breakpoint was position 6594, 6595 or 6596 in intron 8 (FIG. 18C). The same AF-4-MLL genomic breakpoint junction was confirmed in the 291-bp products of two PCRs performed with AF-4 and MLL clonotypic primers. In addition to 5′-cytosine adenine ‘CA’-3′ sequences, whose presence at the breakpoints in both genes precluded more precise assignments, other short homologous sequences in MLL and AF-4 were found to flank the der(4) genomic breakpoint junction (FIG. 18C). The closest repetitive sequences to the der(4) breakpoints in both genes were MERs (FIG. 18C). Depending on the exact positions of the der(11) and der(4) MLL and AF-4 genomic breakpoints, a 4-7 bp region from MLL intron 8 and a 19-22 bp region from AF-4 intron 3 were lost during the translocation.

cDNA panhandle PCR identified an in-frame chimeric transcript from the der(11) chromosome joining MLL exon 8 to AF-4 exon 4. The fusion was detected in six of nine recombination PCR-generated subclones that were sequenced. PCR with MLL- and AF-4-specific primers and sequencing of the 507 bp product confirmed this fusion transcript. RT-PCR with AF-4- and MLL-specific primers gave a 293 base-pair product; the sequence indicated that the der(4) transcript fused AF-4 exon 3 in-frame to MLL exon 9 (data not shown).

Reverse Panhandle PCR Identifies CDK6 as a New Partner Gene of MLL in Complex Translocation Involving MLL. AF-4 and CDK6.

In the infant ALL of patient 38, Southern blot analysis of BamHI-digested DNA from the diagnostic marrow revealed 7.0 kb and 2.0 kb MLL bcr rearrangements (FIG. 19A) (Felix et al., 1997, Blood 90: 4679-4686). Although cytogenetic analysis of this marrow specimen was unsuccessful, panhandle PCR identified an MLL intron 8-AF-4 intron 3 genomic breakpoint junction of a putative der(11) chromosome (Felix et al., 1997, Blood 90: 4679-4686). The MLL breakpoint was position 3802 in intron 8; the AF-4 breakpoint was position 16039 in intron 3 (GenBank accession number AF031403) (Felix et al., 1997, Blood 90: 4679-4686; Example 1). Because PCR with AF-4- and MLL- specific primers designed from this sequence did not identify the predicted der(4) breakpoint junction (data not shown), reverse panhandle PCR was used to identify the genomic breakpoint junction of the other derivative chromosome of this translocation, the presence of which was suggested by the two MLL bcr rearrangements on the Southern blot. The panhandle PCR product size suggested that the 7.0 kb rearrangement was from the putative der(11) chromosome (Felix et al., 1997, Blood 90: 4679-4686). The Southern blot and panhandle PCR product size together predicted a reverse panhandle PCR product of ˜2.0 kb, and a reverse panhandle PCR product of 2241 bp was obtained (FIG. 19A), the sequence of which indicated that the 3′ portion of the MLL bcr had not fused with AF-4 but with the cyclin dependent kinase 6 (CDK6) gene from chromosome band 7q21-q22 (FIG. 19B). The CDK6 intron 2 breakpoint corresponded to position 39,675-39,677 of the genomic clone AC004128. The MLL breakpoint was position 7156-7158 in intron 9, indicating that 3355-3357 bases were lost from MLL in the complex rearrangement. Homologous 5′-Adenine Guanine ‘AG’-3′ sequences in CDK6 and MLL precluded more precise breakpoint assignments, and other short homologous sequences in CDK6 and MLL flanked the breakpoint junction (FIG. 19B). The CDK6-MLL genomic breakpoint junction was confirmed in two independent PCRs by CDK6- and MLL-specific primer mediated amplification of DNA derived from the bone marrow cells of patient 38 at diagnosis and sequencing of the 944-bp product obtained. An MLL exon 8-AF-4 exon 4 fusion transcript was produced as previously described (GenBank accession number AF031404) (Felix et al., 1997, Blood 90: 4679-4686). As demonstrated by direct sequencing, the CDK6-MLL rearrangement produced an in-frame fusion transcript of CDK6 exon 2 with MLL exon 10 (FIG. 19C). The point of fusion in CDK6 corresponded with position 486 of the CDK6 cDNA (GenBank accession no. NM_{—}001259). Although there were no mitoses on cytogenetic analysis of the diagnostic bone marrow (Felix et al., 1997, Blood 90: 4679-4686), the bone marrow karyotype at relapse demonstrated del(7)(q21q31) in addition to the t(4;11) (FIG. 20). No material was available for fluorescence in situ hybridization analysis, but the molecular analyses of the diagnostic marrow were consistent with a 3-way translocation. Genomic DNA from the diagnostic marrow was also analyzed with AF-4- and CDK-6-specific primers to determine if a reciprocal AF-4-CDK6 rearrangement had occurred, but no product was obtained. In addition, no AF-4-CDK6 fusion transcript could be detected.

Discussion

Examination of the genomic breakpoint junctions of both derivative chromosomes is essential to an understanding of the MLL translocation process. It is customary to attempt isolation of the genomic breakpoint junction of the other derivative chromosome by PCR with forward and reverse partner gene- and MLL-derived primers designed based on the der(11) sequence (Felix et al., 1999, Molecular Diagnosis 4: 269-283; Megonigal et al., 2000, Proc Natl Acad Sci, USA 97: 2814-2819) or, in the case of the t(4;11), based on karyotypic evidence of potential involvement of a known partner gene of MLL (Reichel et al., 1999, Cancer Res. 59: 3357-3362; Gillert et al., 1999, Oncogene 18: 4663-4671). This strategy may prove unsuccessful when there are large duplications, deletions, inversions or complex rearrangements, or when the der(11) sequence or the partner gene is unknown. Because MLL has many different partner genes, methodology involving amplification reactions wherein all primers can be derived from MLL sequences broadens the application of such techniques to identify unknown translocation partners. Such methodology includes panhandle PCR and panhandle variant PCR, which have been utilized to examine der(11) genomic breakpoint junctions, and cDNA panhandle PCR, which has been used to investigate der(11) transcripts (Megonigal et al., 2000, Proc Natl Acad Sci, USA 97: 2814-2819; Megonigal et al., 1998, Proc Natl Acad Sci USA 95: 6413-6418; Felix et al., 1997, Blood 90: 4679-4686; Felix et al., 1998, Leukemia 12: 976-981; Megonigal et al., 1997, Proc Natl Acad Sci USA 94: 11583-11588; Megonigal et al., 2000, Proc Natl Acad Sci USA 97: 9597-9602; Pegram et al., 2000, Blood 96: 4360-4362).

As described herein, a reverse panhandle PCR approach, which has features similar to panhandle PCR and panhandle variant PCR, was used to clone the genomic breakpoint junctions of additional derivative chromosomes (Megonigal et al., 2000, Proc Natl Acad Sci, USA 97: 2814-2819; Megonigal et al., 1998, Proc Natl Acad Sci USA 95: 6413-6418; Felix et al., 1997, Blood 90: 4679-4686; Felix et al., 1998, Leukemia 12: 976-981; Megonigal et al., 1997, Proc Natl Acad Sci USA 94: 11583-11588). Stem-loop templates were created in all three genomic methods by BamHI digestion, which created a fragment size amenable to PCR, and ligation of known MLL bcr sequence to the unknown partner sequence in the BamHI fragment (Megonigal et al., 2000, Proc Natl Acad Sci, USA 97: 2814-2819; Megonigal et al., 1998, Proc Natl Acad Sci USA 95: 6413-6418; Felix et al., 1997, Blood 90: 4679-4686; Felix et al., 1998, Leukemia 12: 976-981; Megonigal et al., 1997, Proc Natl Acad Sci USA 94: 11583-11588). All four panhandle PCR approaches may be used for analysis of uncharacterized juxtaposed sequences, leading readily to the identification of known and unknown partner genes in MLL translocations.

The reverse panhandle PCR approach is, however, of greater utility for the identification of breakpoint junction sequences that have been created by complex, multi-step translocation processes.

While both the der(11) and der(4) genomic breakpoint junctions have been amplified with gene-specific primers in many de novo leukemias with t(4;11) (Reichel et al., 1999, Cancer Res. 59: 3357-3362; Gillert et al., 1999, Oncogene 18: 4663-4671; Felix et al., 1999, Molecular Diagnosis 4: 269-283; Reichel et al., 1998, Oncogene 17: 3035-3044), both genomic breakpoint junctions have been characterized in few de novo leukemias with other MLL translocations (Super et al., 1997, Genes, Chromosomes & Cancer 20: 185-195) and few leukemias following anticancer treatment with DNA topoisomerase II inhibitors (Megonigal et al., 2000, Proc Natl Acad Sci, USA 97: 2814-2819; Domer et al., 1995, Leukemia 9: 1305-1312; Strout et al., 1996, Genes, Chromosomes & Cancer 16: 204-210; Lovett et al., Proc Natl Acad Sci USA, In Press). As described herein for the ALL of patient 45, the sequences in de novo cases exhibit regions up to several hundred bases long from MLL and/or from a partner gene on both derivative chromosomes, suggesting duplication events (Reichel et al., 1999, Cancer Res. 59: 3357-3362; Felix et al., 1999, Molecular Diagnosis 4: 269-283; Super et al., 1997, Genes, Chromosomes & Cancer 20: 185-195; Reichel et al., 1998, Oncogene 17: 3035-3044). Deletions of several hundred bases from MLL and from its partner genes have also been observed in de novo cases (Reichel et al., 1999, Cancer Res. 59: 3357-3362; Gillert et al., 1999, Oncogene 18: 4663-4671; Super et al., 1997, Genes, Chromosomes & Cancer 20: 185-195). Studies in which both genomic breakpoint junctions of MLL translocations have been examined in chemotherapy-related leukemias, including that of patient t-120, revealed the presence of more precise interchromosomal DNA recombinations with deletions or duplications of relatively few bases (Megonigal et al., 2000, Proc Natl Acad Sci, USA 97: 2814-2819; Domer et al., 1995, Leukemia 9: 1305-1312; Lovett et al., Proc Natl Acad Sci USA, In Press). The ML-1 cell line (Strout et al., 1996, Genes, Chromosomes & Cancer 16: 204-210), however, which was derived from a patient having chemotherapy-related leukemia, is an exception and possesses imprecise interchromosomal DNA recombinations.

The MLL-AF-4 genomic breakpoint junction in the ALL of patient 38 predicted a reciprocal AF-4-MLL genomic breakpoint junction, but reverse panhandle PCR analysis revealed a CDK6-MLL junction, which resulted from a cryptic, complex, three-way rearrangement involving MLL, AF-4 and CDK6. The ˜226 kb CDK6 gene located at chromosome 7q21-q22 contains 7 exons (Thomas et al., 1999, Mamm Genome. 10: 746-767), which encode a 40 kd protein comprising 325-amino acids (Brotherton et al., 1998, Nature 395: 244-250; Chilosi et al., 1998, Am J Pathol. 152: 209-217). It has been suggested that MLL partner proteins perform general, rather than cell-type specific functions (Ayton et al., 2001, In: Ravid K, Licht J D, eds. Transcription Factors: Normal and Malignant Development of Blood Cells: Wiley-Liss, Inc.). cdk6, for example, is a D-cyclin dependent kinase and a general signaling protein at the G1-S cell cycle transition (Lin et al., 2001, Oncogene 20: 2000-2009). Significantly, cdk6 is also the major cdk in human lymphoid cells (Chilosi et al., 1998, Am J Pathol. 152: 209-217; Lin et al., 2001, Oncogene 20: 2000-2009; Lucas et al., 1995, J Immunol. 154: 6275-6284; Wagner et al., 1998, J Immunol. 161: 1123-1131). In brief, cdk6 is activated by D-cyclin and subsequently phosphorylates and inactivates the Rb protein, thus inhibiting its growth-suppressive functions. Inhibition of Rb functions results in the activation of E2F transcription factors and enables entry into S phase (Lin et al., 2001, Oncogene 20: 2000-2009; Ragione et al., 1997, Leuk Lymphoma 25: 23-35; Sherr, 2000, Cancer Res. 60: 3689-3695).

An in-frame CDK6-MLL fusion transcript was identified in the ALL of patient 38 (FIG. 19C). The corresponding full-length fusion transcript would include the first 123 codons of CDK6 and the last 2476 codons of MLL. As shown in FIG. 19D, the fusion protein was predicted to include the PLSTIRE helixα1,_β_sheets, catalytic cleft and 22 residues from the carboxy-terminal α helices of cdk6 (Brotherton et al., 1998, Nature 395: 244-250), and the zinc fingers, transactivation domain and SET domain of MLL. The term ‘partner gene’ generally refers to a gene whose 3′ sequence is fused to the 5′ sequence of MLL. CDK6 is, however, the first gene to be identified wherein the 3′ sequence of MLL is fused to the 5′ sequence of the ‘partner gene’ (i.e., CDK6) in an MLL translocation.

MLL translocations are thought to be leukemogenic by producing chimeric oncoproteins from the der(11) fusion in which the amino terminus of MLL is joined to the carboxy terminus of the partner protein (Ayton et al., 2001, In: Ravid K, Licht J D eds., Wiley-Liss, Inc.). Murine models have used 5′-MLL-partner-3′ constructs to establish that MLL translocations are leukemogenic (Ayton et al., 2001, In: Ravid K, Licht J D eds., Wiley-Liss, Inc.). Latency to leukemia in these models suggests that additional alterations may also be important. At least one such construct (5′-MLL-FBP17-3′) shows minimal transformation in serial replating assays (Fuchs et al, 2001, Proc Natl Acad Sci, USA 98: 8756-8761). However, experiments on the potential functional contribution of partner-MLL fusion proteins have not been performed. While an MLL-AF-4 transcript was produced (Felix et al., 1997, Blood 90: 4679-4686) and the der(11) gene product is considered critical in leukemogenesis (Ayton et al., 2001, In: Ravid K, Licht J D eds., Wiley-Liss, Inc.), it is possible that the cdk6-MLL fusion protein, predicted by the maintenance of a productive open-reading frame in the CDK6-MLL transcript, may have contributed as well.

The effects of the putative cdk6-MLL fusion protein on cdk6 and MLL function are unknown but cdk6 is a recurrent target for molecular alterations in other forms of cancer. cdk6 overexpression has been observed in T-cell lymphoblastic lymphoma (Chilosi et al., 1998, Am J Pathol. 152: 209-217), T-cell ALL (Chilosi et al., 1998, Am J Pathol. 152: 209-217), natural killer/T-cell nasal lymphoma (Lien et al., 2000, Lab Invest. 80: 893-900) and glioblastoma multiforme (GBM) (Costello et al., 1997, Cancer Res. 57: 1250-1254; Lam et al., 2000, Br J Neurosurg. 14: 28-32), and in squamous cell carcinoma (Timmermann et al., 1997, Cell Growth Differ. 8: 361-370) and neuroblastoma cell lines (Easton et al., 1998, Cancer Res. 58: 2624-2632). p15 and p16, members of the INK4 family of cdk inhibitors, are often deleted in T-cell ALL (reviewed in (Ragione et al., 1997, Leuk Lymphoma 25: 23-35)). p27KIP1 deletions found in ALL may also contribute to cdk6 dysregulation (Komuro et al., 1999, Neoplasia 1: 253-261), since the p27^{Kip1} tumor suppressor protein controls assembly of the cyclin D3-cdk6 complex (Sherr, 2000, Cancer Res. 60: 3689-3695). Splenic lymphomas with villous lymphocytes, which are of B-cell origin, are characterized by t(2;7)(p12;q21) translocations juxtaposing CDK6 to the Ig kappa locus and resulting in increased cdk6 expression (Corcoran et al., 1999, Oncogene 18: 6271-6277). A t(7;21) translocation disrupting CDK6 has also been observed in a splenic marginal zone lymphoma (Corcoran et al., 1999, Oncogene 18: 6271-6277). Thus, the central role of cdk6 in cell cycle progression and its recurrent alteration in human cancer suggest that the CDK6-MLL juxtaposition may have been a cooperating mutation in leukemogenesis in patient 38. G-banded and spectral karyotype analyses have also identified t(7;11)(q22;q23) in infant ALL, MDS, and non-Hodgkin lymphoma (Mitelman et al., 2000, http://cgap.nci.nih.gov/Chromosomes/Mitelman), possibly suggesting that CDK6-MLL junctions will be found in other cases.

Analysis of genomic breakpoint junction sequences and the fusion transcripts resulting from three-way rearrangements provide insights into the translocation process and molecular alterations leading to leukemias with MLL translocations. Other three-way MLL translocations have been identified (Taki et al., 1996, Oncogene 13: 2121-2130; So et al., 2000, Cancer Genet Cytogenet 117: 24-27; Bernasconi et al., 2000, Cancer Genet Cytogenet 116: 111-118; Takahashi et al., 1996, Cancer Genet Cytogenet 88: 26-29). Reverse panhandle PCR is a significant advance for cloning the breakpoint junctions of other derivative chromosomes resulting from MLL translocations, and is ideal for the study of leukemias with complex rearrangements because the necessity of sequence specific primers for the partner genes is obviated. The duplicated MLL and AF-4 sequences in the ALL of patient 45, the small MLL and AF-4 deletions in the treatment-related ALL of patient t-120 and the complex translocation in the ALL of patient 38 suggest that considerable heterogeneity in MLL genomic breakpoint junctions exists as a consequence of variable DNA damage and repair.

### Example 9

Panhandle PCR Strategy to Amplify Genomic Breakpoints in Infant Acute Myelomonocytic Leukemia and Infant Acute Myeloid Leukemia

Translocations of the MLL gene at chromosome band 11q23 are the most common molecular abnormalities in leukemia of infants (reviewed in (Felix, 2000, Hematology 2000: Education Program of the American Society of Hematology, 294-298)). As described above, MLL translocations in infant leukemias are in utero events (Ford et al., 1993, Nature 363: 358-360; Gale et al., 1997, Proc Natl Acad Sci USA 94: 13950-13954; Gill-Super et al., 1994, Blood 83: 641-644; Mahmoud et al., 1995, Med Pediatr Oncol 24: 77-81; Megonigal et al., 1998, Proc Natl Acad Sci USA 95, 6413-6418). MLL has many partner genes that encode proteins of several different types (reviewed in (Ayton and Cleary, 2001, Transcription Factors: Normal and Malignant Development of Blood Cells. Ravid, K. and Licht, J. D. (eds). Wiley-Liss, Inc.; Felix, 2000, Hematology 2000: Education Program of the American Society of Hematology 294-298; Rowley, 1998, Annu Rev Genet 32: 495-519)). Genomic breakpoint junction sequences of MLL chimeric transcripts involving thirty-two partner genes have been described, but others have not yet been cloned (Huret, 2001, Atlas Genet Cytogenet Oncol Haematol, http://www.infobiogen.fr/services/chromcancer/Anomalies/11q23ID1030.html).

Several MLL fusions with nuclear transcription factors (Corral et al., 1996, Cell 85: 853-861; Lavau et al., 1997, EMBO J 16: 4226-37) or with proteins central to transcriptional regulation (Lavau et al., 2000a, EMBO J 19: 4655-64; Lavau et al., 2000b, Proc Natl Acad Sci USA 97: 10984-9) transform hematopoietic progenitors (Lavau et al., 2000a, EMBO J 19: 4655-64; Lavau et al., 2000b, Proc Natl Acad Sci USA 97: 10984-9; Lavau et al., 1997, EMBO J 16: 4226-37), and/or are leukemogenic in transgenic mice (Corral et al., 1996, Cell 85: 853-861) or in mouse models created by retroviral-mediated gene transfer (Lavau et al., 2000a, EMBO J 19: 4655-64; Lavau et al., 1997, EMBO J 16: 4226-37). Whereas many MLL partner proteins have structural motifs of nuclear transcription factors (LAF-4, AF4, AF5α, AF5q31, AF6q21, AF9, AF10, MLL, AF17, ENL, AFX) (Borkhardt et al., 1997, Oncogene 14: 195-202; Chaplin et al., 1995, Blood 86: 2073-2076; Gu et al., 1992, Cell 71: 701-708; Hillion et al., 1997, Blood 9: 3714-3719; Morrissey et al., 1993, Blood 81: 1124-1131; Nakamura et al., 1993, Proc Natl Acad Sci USA 90: 4631-4635; Prasad et al., 1994, Proc Natl Acad Sci USA 91: 8107-8111; Schichman et al., 1994, Proc Natl Acad Sci USA 91: 6236-6239; Taki et al., 1996, Oncogene 13: 2121-2130; Taki et al., 1999a, Proc Natl Acad Sci USA 96: 14535-14540; Tkachuk et al., 1992, Cell 71: 691-700), proteins involved in transcriptional regulation (CBP, ELL, p300) (Ida et al., 1997, Blood 90: 4699-4704; Sobulo et al., 1997, Proc Natl Acad Sci USA 94: 8732-8737; Taki et al., 1997, Blood 89: 3945-3950; Thirman et al., 1994, Proc Natl Acad Sci USA 91: 12110-12114) or, in one case, a nuclear protein of unknown function (AF15q14) (Hayette et al., 2000, Oncogene 19: 4446-50), other MLL partner proteins are found in the cytoplasm (AF1p, AF1q, AF3p21, GMPS, LPP, GRAF, FBP17, ABI-1, GAS7, EEN) (Bernard et al., 1994, Oncogene 9: 1039-1045; Borkhardt et al., 2000, Proc Natl Acad Sci USA 97: 9168-73; Daheron et al., 2001, Genes, Chromosomes & Cancer 31: 382-389; Fuchs et al., 2001, Proc Natl Acad Sci USA 98: 8756-61; Megonigal et al., 2000a, Proc Natl Acad Sci USA 97: 2814-2819; Pegram et al., 2000, Blood 96: 4360-4362; Sano et al., 2000, Blood 95: 1066-1068; So et al., 1997, Proc Natl Acad Sci USA 99: 2563-2568; Taki et al., 1998, Blood 92: 1125-1130; Tse et al., 1995, Blood 85: 650-656) or at the cell membrane (AF6, LARG, GPHN) (Eguchi et al., 2001, Genes, Chromosomes & Cancer 32: 212-21; Kourlas et al., 2000, Proc Natl Acad Sci USA 97: 2145-50; Prasad et al., 1993, Cancer Res 53: 5624-5628).

Septins, for example, are cytoplasmic proteins with roles in cell division, cytokinesis, cytoskeletal filament formation and GTPase signaling (Cooper and Kiehart, 1996, J Cell Biol 134: 1345-8; Field and Kellogg, 1999, Trends Cell Biol 9: 387-94). In AML of infant twins, the hCDCrel (human Cell Division Cycle related) gene at chromosome band 22q11.2 was the first SEPTIN gene identified as a fusion partner of MLL (Megonigal et al., 1998, Proc Natl Acad Sci USA 95: 6413-6418). hCDCrel involvement has been shown in other cases (Tatsumi et al., 2001, Genes, Chromosomes & Cancer 30: 230-235), indicating that MLL-hCDCrel is a recurrent translocation. The MLL Septin-like fusion (MSF) gene at chromosome band 17q25 is a partner gene of MLL in infant and treatment-related leukemias (Osaka et al., 1999, Proc Natl Acad Sci USA 96: 6428-6433; Taki et al., 1999b, Cancer Res 59: 4261-5). See Example 7.

SEPTIN6 is the third SEPTIN family member disrupted by MLL translocations. Herein, the recurrent involvement of SEPTIN6 in two cases of infant AML is described. MLL-SEPTIN6 chimeric transcripts were recently reported in four cases of infant AML (Borkhardt et al., 2001, Genes, Chromosomes & Cancer 32: 82-88; Ono et al., 2002, Cancer Res 62: 333-337), but the genomic breakpoint junctions were not cloned. Together with prior results on the MLL-hCDCrel genomic breakpoint junction in AML of infant twins (Megonigal et al., 1998, Proc Natl Acad Sci USA 95: 6413-6418), results described herein on the genomic sequencing, FISH and SKY characterization, and DNA topoisomerase II cleavage assays of this complex translocation suggest that the MLL gene and SEPTIN family genes are vulnerable to damage and form translocations associated with infant AML.

The following protocols are provided to enable practice of the methods of Example 9.

Materials and Methods Southern Blot Analysis.

The MLL breakpoint cluster region (bcr) was examined in BamHI-digested DNA using the B859 fragment of ALL-1 cDNA (Gu et al., 1992, Cell 71: 701-708).

cDNA Panhandle PCR Analysis of MLL Fusion Transcripts.

First-strand cDNAs were synthesized from 0.5-1 μg of total RNA using oligonucleotides containing MLL exon 5 sequence at the 5′ ends and random hexamers at the 3′ ends (Megonigal et al., 2000a, Proc Natl Acad Sci USA 97: 2814-2819; Megonigal et al., 2000c, Proc Natl Acad Sci USA 97: 9597-9602). Second-strand cDNA synthesis, formation of stem-loop templates, and PCR with MLL-specific primers were as described (Megonigal et al., 2000a, Proc Natl Acad Sci USA 97: 2814-2819; Megonigal et al., 2000c, Proc Natl Acad Sci USA 97: 9597-9602). Products were subcloned by recombination PCR; the subclones were screened by PCR and sequenced (Megonigal et al., 2000a, Proc Natl Acad Sci USA 97: 2814-2819; Megonigal et al., 2000c, Proc Natl Acad Sci USA 97: 9597-9602).

MLL fusion transcripts were confirmed by amplifying 2 μl of the same first-strand cDNAs with the MLL exon 5 sense primer 5′-AGT GAG CCC AAG AAA AAG CA-3′ (SEQ ID NO: 53) corresponding to positions 3973 to 3992 in the HUMHRX cDNA (GenBank no. L04284) and the SEPTIN6 exon 2 antisense primer 5′-GCA CAG GAT GTT GAA GCA GA-3′ (SEQ ID NO: 54) corresponding to positions 134 to 115 in the KIAA0128 cDNA (GenBank no. D50918). The products were gel-purified and sequenced.

1I Panhandle Variant PCR.

Genomic DNA from the leukemia of patient 62 was studied by panhandle variant PCR. Reactions were performed and the products were subcloned by recombination PCR as described (Megonigal et al., 1998, Proc Natl Acad Sci USA 95: 6413-6418). The breakpoint junction was confirmed by PCR with primers 5′-TCT GTT GCA AAT GTG AAG GC-3′ (SEQ ID NO: 55) corresponding to positions 2288-2307 in MLL intron 6 (GenBank no. U04737) and 5′-TTT TTG AGA CGG ATT CCC AC-3′ (SEQ ID NO: 56) corresponding to positions 3557 to 3576 in SEPTIN6 intron 1 (GenBank nos. AL355348.28; AC005052.2).

Spectral Karyotype Analysis (SKY).

Metaphases for SKY were prepared from 10⁷ viably frozen bone marrow cells that were thawed and cultured for 24,48 and 72 h in RPMI 1640 medium (Gibco BRL, Gaithersburg, Md.) supplemented with 20% FBS, 10% Giant-Cell-Tumor-Conditioned Medium (Origen™ Gaithersburg, Md.), 10 ng/ml IL-3 (Boehringer Mannheim, Indianapolis, Ind.), 50 ng/mL SCF (Boehringer Mannheim) and 50 ng/mL Flt-3 (R&D Systems, Minneapolis, Minn.). Chromosomes were harvested and metaphase spreads were prepared following standard procedures (Roulston and Le Beau, 1997, The AGT cytogenetics laboratory manual, Barch et al. (eds). Lippincott-Raven: Philadelphia, Pa., 325-72). Twenty-four differentially labeled chromosome-specific painting probes were simultaneously hybridized onto metaphase chromosomes as described (Schröck et al., 1996, Science 273: 494-497). Probe preparation, slide pretreatment, hybridization and detection were performed using established protocols (Macville et al., 1997, Histochem Cell Biol 108: 299-305). Ten metaphases were imaged using the SpectraCube™SD200 system (Applied Spectral Imaging, Carlsbad, Calif.) connected to an epifluorescence microscope (DMRXA, Leica Microsystems, Wetzlar, Germany) and analyzed together with the corresponding inverted DAPI images using SkyView™ v.1.2.04 software (Applied Spectral Imaging). The karyotype was interpreted according to the guidelines for cytogenetic nomenclature of the ISCN 1995 (Mitelman, 1995, An International System for Human Cytogenetic Nomenclature. Karger, Inc., Basel).

Fluorescence in situ Hybridization Analysis (FISH).

FISH was performed according to standard procedures using chromosome painting probes for chromosomes 3 and 11, a centromere probe for X (Spectrum Acqua, Vysis, Downers Grove, Ill.) and a DNA probe for MLL (Ventana Medical Systems, Tuscon, Ariz.). Images were acquired using Leica Q-FISH software (Leica Imaging Systems, Cambridge, UK).

DNA Topoisomerase II in vitro Cleavage Assay.

A DNA fragment spanning MLL intron 7/exon 8 positions 2490 to 3077 and containing the normal homologue of the MLL genomic breakpoint in the AML of patient 62, was subcloned into pBluescript II SK (Stratagene; La Jolla, Calif.). The singly 5′ end-labeled, double-stranded DNA substrate was prepared from the plasmid as described (Lovett et al., 2001, Proc Natl Acad Sci USA 98: 9802-9807). Twenty-five ng of substrate DNA were incubated with human DNA topoisomerase IIα, ATP and MgCl₂ either in the absence or presence of 20 μM etoposide and incubated using reaction conditions previously described for other cleavage assays (Lovett et al., 2001, Proc Natl Acad Sci USA 98: 9802-9807). Covalent complexes were irreversibly trapped by adding SDS, without or following incubation for 10 min at 65° C., the latter to evaluate heat stability (Lovett et al., 2001, Proc Natl Acad Sci USA 98: 9802-9807). The cleavage products were deproteinized and electrophoresed in a denaturing polyacrylamide gel in parallel with a dideoxy sequencing ladder to map the sites of cleavage (Lovett et al., 2001, Proc Natl Acad Sci USA 98: 9802-9807).

Results

Case Histories. Patient 62 presented at 20 months of age with hepatosplenomegaly, massive adenopathy, anemia, thrombocytopenia, a WBC of 397×10⁹/L and leukemia of the central nervous system (CNS). The bone marrow was 91% replaced by French-American-British (FAB) M4 blasts that expressed CD33, CD15, CD11b and HLA DR. The original G-banded karyotype in 8 of 8 metaphases was 47,X,t(X;3)(q22;p21)ins(X;11)(q22;q13q25), +6,del(11)(q13). The patient died of infectious complications during induction.

Clinicopathologic features of patient 23, who presented at age 10 months with a WBC count of 13.4×10⁹/L, 13% circulating blasts and pancyotpenia, have been described (Felix et al., 1998, J Pediatr Hematol/Oncol 20: 299-308). The marrow morphology was FAB M2. The immunophenotype was CD11+, CD13+, CD15+, CD33+; no lymphoid antigens were expressed. The karyotype in 25 of 30 metaphases revealed 46,Y,t(X;11)(q22;q23). Four months later, only partial remission had been achieved after treatment according to protocol CCG 2891 (Woods et al., 1996, Blood 87: 4979-4989); the karyotype was 45,Y,t(X;11)(q22;q23),-7[1]/46,XY[23]. The patient was removed from protocol and underwent a haploidentical transplant with his mother's marrow. He remains disease free with chronic graft-versus-host disease 7 years from diagnosis.

Molecular and Cytogenetic Characterization of a Complex MLL-SEPTIN6 Rearrangement.

Although the G-banded karyotype of the AML of patient 62 did not show involvement of band 11q23, Southern blot analysis of the MLL bcr was performed because the morphology was FAB M4. MLL bcr rearrangement suggested that the t(X;3)(q22;p21)ins(X;11)(q22;q13q25) disrupted MLL (FIG. 21A). The single rearrangement was consistent with loss of the 3′ portion of the MLL bcr during the translocation.

cDNA panhandle PCR identified the partner gene of MLL (FIG. 21B). Sequencing of recombination PCR-generated subclones from cDNA panhandle PCR revealed two types of MLL-containing transcripts. The majority of subclones contained an in-frame fusion of MLL exon 7 to exon 2 at position 24 of the 4612 bp SEPTIN6 cDNA from chromosome band Xq24 (GenBank no. D50918); two subclones indicated incomplete processing of this transcript (FIG. 21B). The second type of transcript contained MLL sequence only and was also incompletely processed (FIG. 21B). Amplification of the same first-strand cDNA with MLL and SEPTIN6-specific primers and sequencing of the 357-bp product confirmed the fusion transcript (data not shown). There was no evidence of alternative splicing of the fusion transcript from either cDNA panhandle PCR or PCR with gene-specific primers.

The corresponding MLL-SEPTIN6 genomic breakpoint junction was isolated by panhandle variant PCR. The product size (FIG. 21C) was consistent with the ˜3.3 kb MLL bcr rearrangement on the Southern blot (FIG. 21A). The MLL genomic breakpoint was position 2595 in intron 7; the SEPTIN6 genomic breakpoint was position 3321 of 17,407 in intron 1 (GenBank no. AC005052.2) (FIG. 21C). The MLL breakpoint was near Alu Y and Alu Jb repeats; the SEPTIN6 breakpoint was near an AluY repeat (FIG. 21C). Several 2- to 5-base homologies were present near the breakpoints in both genes (FIG. 21C). The sequence of the 564-bp product obtained by PCR with MLL- and SEPTIN6-specific primers confirmed the breakpoint junction (data not shown).

Spectral karyotype analysis (SKY) and fluorescence in situ hybridization analysis (FISH) allowed visualization of the chromosomal abnormalities and the translocation. SKY analysis of ten metaphase cells indicated a more complex translocation and disruption of band 11q23. The spectral karyotype was 47,X,der(X)t(X;11)(q22;q23)t(3;11)(p21;q12),der(3)t(3;11)(p21;q23) t(X;11)(q22;q25),+6,der(11)del(11)(q12?qter) (FIG. 22).

FISH analysis of 12 of 14 metaphase cells with the MLL probe (Ventana) detected one MLL signal on the normal chromosome 11 and signals on the der(X) and the der(3), confirming MLL disruption. The location of the signal on the der(3) at the interface between material from chromosome 11 and chromosome 3 suggested that the MLL-SEPTIN6 fusion was created on the der(X) and that, cytogenetically, no reciprocal fusion could be created through this aberration (FIG. 23). Since the single MLL bcr rearrangement on Southern blot analysis was consistent with deletion of the 3′ bcr, detection of split MLL signals on the der (X) and der(3) chromosomes by FISH suggested that the MLL sequences on the der(3) chromosome were distal to the bcr.

MLL Genomic Breakpoint in Complex Rearrangement is a DNA Topoisomerase II Cleavage Site.

A DNA topoisomerase II in vitro cleavage assay was performed to determine the feasibility of DNA topoisomerase II cleavage at the MLL bcr translocation breakpoint in the AML of patient 62. MLL position 2595, which was the translocation breakpoint, was the 5′ side or −1 position of a naturally-occurring, enzyme-only cleavage site. The DNA topoisomerase II inhibitor etoposide enhanced cleavage at this site 1.2-fold over cleavage without drug (FIG. 24). Although stronger cleavage was discerned at several sites in the substrate both with and without drug and position 2595 did not appear to be a highly preferred cleavage site, detection of cleavage after heating to 65° C. indicates stability of the cleavage complexes formed at this position (FIG. 24).

Detection of MLL-SEPTIN6 Fusion Transcript in Infant AML with t(X:11)(q22,q23).

In the FAB M2 AML of patient 23, the t(X;11)(q22;q23) translocation was the only clonal abnormality detected by the karyotype (Felix et al., 1998, J Pediatr Hematol/Oncol 20: 299-308). Southern blot analysis of the MLL bcr showed two rearrangements consistent with an MLL translocation (FIG. 25A) (Felix et al., 1998, J Pediatr Hematol/Oncol 20: 299-308). cDNA panhandle PCR revealed the fusion transcript with MLL exon 8 fused in-frame to SEPTIN6 exon 2. The point of fusion at position 24 of the 4612 bp SEPTIN6 cDNA (GenBank no. D50918) was the same as in the fusion transcript in the AML of patient 62 (FIG. 25B). The presence of SEPTIN6 intron 3 sequence in the majority of subclones was consistent with a related, incompletely processed transcript (FIG. 25B). Additional subclones contained MLL exon 5 and 6 sequence only (FIG. 25B). Amplification of the same first-strand cDNA with MLL- and SEPTIN6-specific primers and sequencing of the 471 bp product confirmed the fusion transcript (data not shown). There was no evidence of alternative splicing of the fusion transcript.

Discussion

Cytogenetic detection of chromosome band Xq22 and Xq24 as novel chromosomal partners of band 11q23 in AML of infants and young children has been observed by several groups in recent years (Felix et al., 1998, J Pediatr Hematol/Oncol 20: 299-308; Harrison et al., 1998, Leukemia 12: 811-822; Mitelman et al., 2001, http://cgap.nci.nih. gov/Chromosomes/Mitelman; Nakata et al., 1999, Leukemia Res 23: 85-88) and the identification of MLL involvement by Southern blot analysis in two of these cases (Felix et al., 1998, J Pediatr Hematol/Oncol 20: 299-308; Nakata et al., 1999, Leukemia Res 23: 85-88) predicted that one or more partner genes of MLL would be identified in these regions of chromosome Xq.

As described herein, the SEPTIN6 gene has been identified as the gene partner in a cryptic MLL rearrangement in a case of FAB M4 infant AML in which the original G-banded karyotype suggested involvement of band Xq22 but not band 11q23, and in a second case of FAB M2 infant AML with t(X;11)(q22;q23) (Felix et al., 1998, J Pediatr Hematol/Oncol 20: 299-308). In-frame transcripts produced as a consequence of the MLL translocations were shown to comprise a 5′-MLL exon 7-SEPTIN6 exon 2-3′ fusion in one case and a 5′-MLL exon 8-SEPTIN6 exon 2-3′ fusion in the other.

In another recently reported case of infant AML of FAB M2 morphology with cryptic rearrangement of band 11q23, FISH identified ins(X;11)(q24;q23q23), which was also associated with a 5′-MLL exon 8-SEPTIN6 exon 2-3′ fusion transcript (Borkhardt et al., 2001, Genes, Chromosomes & Cancer 32: 82-88). cDNA panhandle PCR characterization of three additional cases of FAB Ml or FAB M2 infant AML with in-frame 5′-MLL exon 7-SEPTIN6 exon 2-3′, 5′-MLL exon 8-SEPTIN6 exon 2-3′, or alternatively spliced 5′-MLL exon 8-SEPTIN6 exon 2-3′ and 5′-MLL exon 7-SEPTIN6 exon 2-3′ fusion transcripts, has recently been described (Ono et al., 2002, Cancer Res 62: 333-337). The karyotype suggested t(5;11)(q13;q23) and add(X)(q22) in one case (Ono et al., 2002, Cancer Res 62: 333-337). In the second case the karyotype was normal but FISH unmasked the abnormality ins(X;11)(q22-24;q23) (Ono et al., 2002, Cancer Res 62: 333-337). The cytogenetic abnormality in the third case was described as add(X)(q2?),del(11q?) (Ono et al., 2002, Cancer Res 62: 333-337). These results further demonstrate the utility of cDNA panhandle PCR (Megonigal et al., 2000b, Proc Natl Acad Sci USA 97: 2814-9; Megonigal et al., 2000d, Proc Natl Acad Sci USA 97: 9597-9602) for partner gene identification in complex rearrangements. Recently, SEPTIN6 was annotated at chromosome band Xq24 in the human genome project (http:genome.ucsc.edu). Molecular detection of MLL-SEPTIN6 transcripts in the two leukemias with cytogenetic Xq22 breakpoints, as described herein, and in the cases with cytogenetic Xq24 breakpoints described by others (Borkhardt et al., 2001; Ono et al., 2002) reveal the recurrent nature of this translocation. These studies also underscore the technical obstacles associated with precise cytogenetic breakpoint definition in leukemias with MLL-SEPTIN6 rearrangements. Six other cases with simple or complex cytogenetic rearrangements of chromosome bands 11q23 and Xq22 or Xq24 have also been reported (Mitelman et al., 2001, http://cgap.nci.nih.gov/Chromosomes/Mitelman), which may prove to involve MLL and SEPTIN6.

Unlike most MLL translocations in which the 5′-MLL-PARTNER GENE-3′ genomic breakpoint junction is created on the der(11) chromosome, combined SKY and FISH analyses of the AML of patient 62 showed that the 5′-MLL-SEPTIN6-3′ genomic breakpoint junction was not on the der(11) chromosome, but on the der(X), where the partner gene resides. Similarly, in two of the recently reported cases of infant AML described above, FISH suggested that the 5′ portion of MLL had been inserted into the X chromosome and that the genomic breakpoint junction from which the MLL-SEPTIN6 transcript was produced was on the der(X) (Borkhardt et al., 2001, Genes, Chromosomes & Cancer 32: 82-88; Ono et al., 2002, Cancer Res 62: 333-337).

In the leukemia of patient 62, the complex translocation suggested damage to the genome. Therefore, the corresponding MLL-SEPTIN6 genomic breakpoint junction was studied in detail. DNA topoisomerase II has been implicated in the DNA damage leading to MLL translocations because of epidemiological associations of chemotherapeutic and dietary DNA topoisomerase II inhibitors, respectively, with treatment-related and infant acute leukemias (Ross, 1998, Int J Cancer Suppl 11: 26-28; Ross et al., 1996, Cancer Causes and Control 7: 581-590; Smith et al., 1999, J Clin Oncol 17: 569-577).

To investigate the relationship between functional DNA topoisomerase II cleavage sites and the MLL genomic breakpoint, in vitro DNA topoisomerase II cleavage assays were performed. DNA topoisomerase II catalyzes transient and reversible cleavage and religation of both strands of the double helix (Fortune and Osheroff, 2000, Prog Nucleic Acid Res and Molecular Biology 64: 221-53). Etoposide decreases the religation rate and is often used in these assays to enhance the detection of the cleavage complexes, which otherwise are inherently unstable and thus more difficult to detect due to their transient nature (Fortune and Osheroff, 2000, Prog Nucleic Acid Res and Molecular Biology 64: 221-53). The detection of heat-stable cleavage complexes at position 2595 even without drug enhancement reveals that the translocation breakpoint sequence is a naturally-occurring site of DNA topoisomerase II cleavage that is resistant to religation. The stability of the broken DNA may be critically relevant to the frequency of translocation detected in this chromosomal region.

Nine mammalian SEPTIN family members have been identified to date (Kinoshita et al., 2000, J Comp Neur 428: 223-239). Human analogues for most of the nine can be found in GenBank. The SEPTIN genes comprise a gene family in which several different members can fuse with MLL. The characterization of identical, non-constitutional 5′-MLL-hCDCrel-3′ genomic breakpoint junction sequences in infant twins established that MLL translocations in infant AML are in utero events (Megonigal et al., 1998, Proc Natl Acad Sci USA 95: 6413-6418). See Example 7. The MLL-hCDCrel genomic breakpoint junction sequence in the AMLs of the infant twins contained evidence of DNA damage and repair (Megonigal et al., 1998, Proc Natl Acad Sci USA 95: 6413-6418) similar to that in the MLL-SEPTIN6 genomic breakpoint junction sequence in the AML of patient 62. That hCDCrel, MSF(AF-17q25), and SEPTIN6 are all disrupted by MLL translocations (Borkhardt et al., 2001, Genes, Chromosomes & Cancer 32: 82-88; Megonigal et al., 1998, Proc Natl Acad Sci USA 95: 6413-6418; Ono et al., 2002, Cancer Res 62: 333-337; Taki et al., 1999b, Cancer Res 59: 4261-5; Tatsumi et al., 2001, Genes, Chromosomes & Cancer 30: 230-235) suggests that SEPTIN family members are particularly vulnerable to damage and recombination to form MLL translocations associated with infant AML. LAF-4 is the only other gene family with three members (LAF-4, AF4, AF5q31) that fuse with MLL (Ayton and Cleary, 2001, Transcription Factors: Normal and Malignant Development of Blood Cells. Ravid, K. and Licht, J. D. (eds). Wiley-Liss, Inc.; Huret, 2001, Atlas Genet Cytogenet Oncol Haematol, http://www.infobiogen.fr/services/chromcancer/Anomalies/11q23ID1030.html; Nilson et al., 1997, Br J Haematol 98: 157-169; Taki et al., 1999a, Proc Natl Acad Sci USA 96: 14535-14540).

First identified in budding yeast and later in Drosophila, Septin proteins are believed to be important in septation, cell division, cytokinesis, vesicle trafficking and exocytosis (Beites et al., 2001, Methods Enzymol 329: 499-510; Kartmann and Roth, 2001, J Cell Sci 114(Pt 5): 839-844; Kinoshita et al., 2000, J Comp Neur 428: 223-239). Although their roles in mammalian cells are incompletely understood, the SEPTIN genes all encode GTP binding proteins with a central, conserved GTPase domain, a variable N-terminal extension domain and a C-terminal coiled coil. The Septin proteins are thought to function in heteropolymeric complexes comprising multiple Septin molecules in the cytoskeleton (Cooper and Kiehart, 1996, J Cell Biol 134: 1345-8; Field and Kellogg, 1999, Trends Cell Biol 9: 387-94; Kinoshita et al., 2000, J Comp Neur 428: 223-239). Murine Septin6 expression is detected in synaptic vesicles in specific regions of the brain (Kinoshita et al., 2000, J Comp Neur 428: 223-239). In the human, several alternatively spliced SEPTIN6 transcripts are differentially expressed in adult and fetal tissues (Ono et al., 2002, Cancer Res 62: 333-337).

The complex rearrangement described herein, whereby the MLL and SEPTIN6 genes have undergone molecular rearrangement involving chromosomes 3, X and 11, is a novel translocation event. The fusion transcripts in both leukemias studied herein and in those reported by others (Borkhardt et al., 2001, Genes, Chromosomes & Cancer 32: 82-88; Ono et al., 2002, Cancer Res 62: 333-337) joined 5′ MLL sequences in-frame with SEPTIN6 exon 2, 5′ in the coding sequence of this gene. Predicted fusion proteins from the MLL-SEPTIN6 translocations contain the N-terminal AT-hook, DNA methyltransferase, and repression domains of MLL and all three domains of Septin 6. Identification of the cellular localization of the resultant fusion proteins will provide significant insight into their role in leukemogenesis.

The identification of three SEPTIN family members as partner genes of MLL suggests an important common pathway to leukemogenesis in AML with these translocations. The high WBC count, organomegaly and myelomonocytic morphology in the AML of patient 62 are archetypal features of leukemias with MLL translocations, but the FAB M2 morphology of the AML of patient 23 and FAB M1 and FAB M2 morphologies observed in other cases (Borkhardt et al., 2001, Genes, Chromosomes & Cancer 32: 82-88; Ono et al., 2002, Cancer Res 62: 333-337) indicate heterogeneity in presenting features. One patient in this study and two of the four reported patients (Borkhardt et al., 2001, Genes, Chromosomes & Cancer 32: 82-88; Ono et al., 2002, Cancer Res 62: 333-337) have survived, suggesting that prognosis for such patients may also be heterogeneous in nature. The role of SEPTIN family aberrations may extend to other cancers, since MSF loss of heterozygosity is frequently observed in cancers of the ovary and breast (Russell et al., 2000, Cancer Res 60: 4729-4734).

### Example 10

Panhandle PCR Strategy is Applicable to Analyses of Human Cancers with Genomic Translocations

Table 1 is provided to exemplify further aspects of the present invention. In view of the enormous database available regarding the association of genomic translocations and human cancers (Mitelman et al., 1997, Nature Genetics Spec. Issue: 417-474; http://cgap.nci.nih.gov/Chromosomes/Mitelman; http://www.wiley.co.uk/products/subject/life/mitelman/mitord.htm), the translocations listed in Table 1 are provided to exemplify a subset of such translocations and should not viewed as a comprehensive list of translocations for which the methods of the present invention may be applied. The methods of the present invention may be used to diagnose any cancer, associated with genomic translocations, in a patient. Accurate diagnosis of a tumor, which includes reverse panhandle PCR-mediated identification of translocations, in addition to standard pathological and histochemical analyses, provides the attending physician with a broader spectrum of clinical information upon which to base a therapeutic regime for the efficacious treatment of a cancer patient. Moreover, the enhanced sensitivity conferred by the reverse panhandle PCR methods of the present invention enables the physician to monitor a patient's response to a therapeutic regime, as cancer regression may be measured by reduction in the level of oncogenic transcript produced by a translocation event. By virtue of the sensitivity of reverse panhandle PCR-mediated identification of translocations, the methods of the present invention also provide ideal tools for the detection of minimal disease prior to treatment and/or minimal residual disease following treatment. Such tools provide an early warning system for relapse of primary disease or onset of a treatment-related disease, which may be detected by monitoring for translocation events known to be associated with particular therapeutic regimes.

In another application, the methods of the present invention may also be used to identify the partner genes in translocation events wherein only one of the two partner genes has been identified. The identification of the second partner gene and/or the nucleic acid sequences flanking the breakpoint junction provides information that may be used to define novel biological targets for therapeutic intervention (Alcalay et al., 2001, Oncogene 20: 5680-5694; Cripe and Mackall, 2001, Ped. Oncol. In 21^{st} Century15:657-675). The unique sequence at the breakpoint junction, for example, may be targeted at either the DNA or RNA level by sequence-specific molecules. In the event that the breakpoint junction encodes a novel antigenic epitope, immunotherapy methods could be directed to such breakpoint junction-specific epitope(s).

TABLE 1
Chromosomal translocations associated with cancers

| | Chromosomal | | |
|---|---|---|---|
| Cancer Type | Abnormality | Genes Involved | Reference |
| Burkett's | t(8; 14)(q24; q32) | c-myc/IgH | Weill Medical College |
| Lymphoma (BL) | | | of Cornell University. |
| | | | http://edcenter.med.cornell. |
| | | | edu/CUMC PathNotes/ |
| | | | Neoplasia/Neopla- |
| | | | sia 08.html |
| Burkett's | t(8; 22) | c-myc/Ig kappa | Weill Medical College |
| Lymphoma (BL) | | | of Cornell University. |
| | | | http://edcenter.med.cornell. |
| | | | edu/CUMC PathNotes/ |
| | | | Neoplasia/Neopla- |
| | | | sia 08.html |
| Burkett's | t(8; 2) | c-myc/Ig lambda | Weill Medical College |
| Lymphoma (BL) | | | of Cornell University. |
| | | | http://edcenter.med.cornell. |
| | | | edu/CUMC PathNotes/ |
| | | | Neoplasia/Neopla- |
| | | | sia 08.html |
| Chronic | t(9; 22)(q34; q11) | c-abl/bcr | Weill Medical College |
| Myelogenous | | | of Cornell University. |
| Leukemia (CML) | | | http://edcenter.med.cornell. |
| | | | edu/CUMC PathNotes/ |
| | | | Neoplasia/Neopla- |
| | | | sia 08.html; Fioretos et |
| | | | al., 2001, Genes |
| | | | Chromosomes Cancer 32: |
| | | | 302-310. |
| Acute Myeloid | t(7; 11)(p15; p15) | HOXA9/NUP98 | Borrow et al., 1996, nat |
| Leukemia (AML) | | | Genet 12(2): 159-67. |
| Acute Myeloid | t(12; 15)(p13; q25) | ETV6/TRKC | Eguchi et al., 1999, |
| Leukemia (AML) | | | Blood 93(4): 1355-63. |
| Chronic | t(5; 12)(q33; p13) | TEL-PDGFβR | Tomasson et al., 1999, |
| Myelomonocytic | | | Blood 93(5): 1707- |
| Leukemia (CMML) | | | 1714. |
| Acute | t(1; 22)(p13; q13) | | Mitelman et al., 1997, |
| Megakaryoblastic | | | Nature Genetics Special |
| Leukemia (AML- | | | Issue: 417-474. |
| M7) | | | |
| Acute | t(12; 21)(p13; q22) | ETV6/AML1 | Harrison C J. 2000, |
| Lymphoblastic | | | Baillieres Best Pract |
| Leukemia (ALL) | | | Res Clin Haematol 13(3): |
| | | | 427-39. |
| Acute | t(8; 21)(q22; q22) | | Rege et al., 2000, Leuk |
| Lymphoblastic | | | Lymphoma 40(1-2): |
| Leukemia (ALL) | | | 67-77. |
| Acute pre-B-cell | t(1; 19)(q23; p13.3) | Pbx1(Prl)/E2A | Saltman et al., 1990, |
| Leukemia | | | Genes Chromosomes |
| | | | Cancer 2(4): 259-65; |
| | | | Mellentin et al., 1990, |
| | | | Genes Chromosomes |
| | | | Cancer 2(3): 239-47; |
| | | | Nourse et al., 1990, |
| | | | Cell 60(4): 535-45; |
| | | | Mellentin et al., 1989, |
| | | | Science 246(4928): |
| | | | 379-82; Kamps et al., |
| | | | 1990, Cell 60(4): 547- |
| | | | 55; Kamps et al., 1991, |
| | | | Genes Dev 5(3): 358- |
| | | | 68. |
| T-Cell All | t(7; 19)(q34; p13) | LYL1 | Saltman et al., 1990, |
| | | | Genes Chromosomes |
| | | | Cancer 2(4): 259-65. |
| T-Cell All | t(1; 14)(p32; q11) | tal-1 | Chen et al., 1990, |
| | | | EMBO J 9(2): 415-24; |
| | | | Brown et al., 1990, |
| | | | EMBO J 9(10): 3343- |
| | | | 51; Chen et al., 1990, J |
| | | | Exp Med 172(5): 1403- |
| | | | 8. |
| SUP-T13 (T-All | t(11; 19)(q23; p13) | | Saltman et al., 1990, |
| Cell Lines | | | Genes Chromosomes |
| | | | Cancer 2(4): 259-65. |
| SUP-T8a (T-All | t(4; 19)(q21; p13) | | Saltman et al., 1990, |
| Cell Lines | | | Genes Chromosomes |
| | | | Cancer 2(4): 259-65. |
| 8p11 MPS | t(8; 13)(p11; q12) | FGFR1/ZNF198 | Fioretos et al., 2001, |
| (Myeloproliferative | | | Genes Chromosomes |
| Syndromes (MPSs)) | | | Cancer 32: 302-310; |
| | | | Xiao et al., 1998, |
| | | | Nature Genetics 18(1): |
| | | | 84. |
| 8p11 MPS | t(8; 9)(p11; q34) | FGFR1/CEP110 | Fioretos et al., 2001, |
| (Myeloproliferative | | | Genes Chromosomes |
| Syndromes (MPSs)) | | | Cancer 32: 302-310. |
| 8p11 MPS | t(8; 6)(p11; q27) | FGFR1/FOP | Fioretos et al., 2001, |
| (Myeloproliferative | | | Genes Chromosomes |
| Syndromes (MPSs)) | | | Cancer 32: 302-310. |
| 8p11 MPS-like | t(8; 22)(p11; q11) | FGFR1/BCR | Fioretos et al., 2001, |
| (Myeloproliferative | | | Genes Chromosomes |
| Syndromes (MPSs)) | | | Cancer 32: 302-310. |
| 8p11 MPS-like | t(8; 13)(q10; p10) | | Behringer et al., 1995, |
| (Myeloproliferative | | | Leukemia 9(6): 988-92. |
| Syndromes (MPSs)) | | | |
| Squamous Cell | t(11)(q13) | | Akervall et al., 2002, |
| Carcinoma of Head and | | | Int J Oncol 20(1): 45- |
| Neck (SCCHN) | | | 52. |
| Alveolar | t(2; 13)(q35; q14) | PAX3/FKHR | Barr, 2001, |
| Rhabdomyosarcoma | | | Oncogene 20(40): 5736-46; |
| | | | Ayalon et al., 2001, |
| | | | Growth Horm IGF |
| | | | Res 11(5): 289-297; Galili |
| | | | et al., 1993, Nat Genet 5: |
| | | | 230-235; Shapiro et |
| | | | al., 1993, Cancer Res 53: |
| | | | 5108-5112. |
| Alveolar | t(1; 13)(p36; q14) | PAX7/FKHR | Barr, 2001, |
| Rhabdomyosarcoma | | | Oncogene 20(40): 5736-46; |
| (ARMS) | | | Davis et al., 1994, Cancer |
| | | | Res 54: 2869-2872. |
| Embryonal | t(1; 2, 8, 12 or | | Gordon et al., 2001, |
| Rhabdomyosarcoma | 13)(p11- | | Med Pediatr Oncol 36(2): |
| (ERMS) | q11; variable) | | 259-67. |
| Papillary | t(10; 12)(q11; p13.3) | RET/ELKS | Nakata et al., 1999, |
| Thyroidcarcinoma | | | Genes Chromosomes |
| (PTC) | | | Cancer 25(2): 97-103; |
| | | | Yokota et al., 2000, J |
| | | | Hum Genet 45(1): 6- |
| | | | 11. |
| Papillary | t(10; 17)(q11.2; q23) | RET/PTC2 | Sozzi et al., 1994, |
| Thyroidcarcinoma | | | Genes Chromosomes |
| (PTC) | | | Cancer 9(4): 244-50. |
| Papillary | t(7; 10)(q32; q11.2) | PTC6/RET | Salassidis et al., 2000, |
| Thyroidcarcinoma | | | Cancer Res 60(11): |
| (PTC) | | | 2786-9. |
| Papillary | t(1; 10)(p13; q11.2) | PTC7/RET | Salassidis et al., 2000, |
| Thyroidcarcinoma | | | Cancer Res 60(11): |
| (PTC) | | | 2786-9. |
| Papillary | t(10; 14)(q11.2; q22.1) | RET/KTN1(PTC8) | Salassidis et al., 2000, |
| Thyroidcarcinoma | | | Cancer Res 60(11): |
| (PTC) | | | 2786-9. |
| Papillary | t(10; 18)(q11; q21- | RET/RFG8 | Klugbauer et al., 2000, |
| Thyroidcarcinoma | 22) | RFG8/RET | Cancer Res 60(24): |
| (PTC) | | | 7028-32. |
| Multifocal | t(3; 5)(q12; p15.3) | | Smit et al., 2001, Clin |
| Follicular Variant of | | | Endocrinol (Oxf) 55(4): |
| PTC | | | 543-8. |
| Follicular Adenoma | t(X; 10)(p22; q24) | | van Zelderen-Bhola et |
| of Thyroid | | | al., 1999, Cancer Genet |
| | | | Cytogenet 112(2): 178- |
| | | | 80. |
| Follicular Adenoma | t(1; 10)(q21; q11) | | van Zelderen-Bhola et |
| of Thyroid | | | al., 1999, Cancer Genet |
| | | | Cytogenet 112(2): 178- |
| | | | 80. |
| Acute | t(15; 17) | RARα/PML | Pandolfi, 2001, |
| Promyelocytic | | | Oncogene 20: 5726- |
| Leukemia (APL) | | | 5735; Kastner et al., |
| | | | 1992, EMBO J 11(2): |
| | | | 629-42. |
| Acute | t(11; 17)(q23; q21) | RARα/PLZF | Chen et al., 1994, Proc. |
| Promyelocytic | | | Natl. Acad. Sci. 91: |
| Leukemia (APL) | | | 1178-1182; Zhang et |
| | | | al., 1999, Proc. Natl. |
| | | | Acad. Sci. 96: 11422- |
| | | | 27. |
| Acute | t(5; 17)(q32; q12) | RARα/NPM | Redner et al., 1996, |
| Promyelocytic | t(5; 17)(q35; q21) | | Blood 87(3): 882-6; |
| Leukemia (APL) | | | Hummel et al., 1999, |
| | | | Oncogene 18(3): 633- |
| | | | 41. |
| Acute | t(11; 17)(q13; q12- | RARα/NuMA | Wells et al., 1997, Nat |
| Promyelocytic | q21.1) | | Genet 17(1): 109-13. |
| Leukemia (APL) | | | |
| Acute | (17; 17) | RARα/STAT5b | Arnould et al., 1999, |
| Promyelocytic | [rearrangement] | | Human Mol Genet 8(9): |
| Leukemia (APL) | | | 1741-9. |
| (T-Cell) Leukemia | t(17; 19) | E2A/HLF | Seidel and Look, 2001, |
| | | | Oncogene 20(40): |
| | | | 5718-25. |
| Anaplastic Large- | t(2; 5)(p23; q35) | NPM/ALK | Morris et al., 1994, |
| Cell Lymphoma | | | Science 263(5151): |
| (ALCL) | | | 1281-4; Bullrich et al., |
| | | | 1994, Cancer Res 54(11): |
| | | | 2873-7. |
| Anaplastic Large- | t(1; 2)(q25; p23) | TPM3/ALK | Lamant et al., 1999, |
| Cell Lymphoma | | | Blood 93(9): 3088-95. |
| (ALCL) | | | |
| Anaplastic Large- | t(2; 3)(p23; q21) | TFG/ALK | Hernandez et al., 1999, |
| Cell Lymphoma | | | Blood 94(9): 3265-8; |
| (ALCL) | | | Rosenwald et al., 1999, |
| | | | Blood 94: 362-4. |
| Malignant | T(5; 6)(q35; p21) | IgH/ | Gogusev et al., 1990, |
| Histiocytosis (MH) | | | Int J Cancer 46(1): |
| | | | 106-12; Nezelof et al., |
| | | | 1992, Semin Diagn |
| | | | Pathol 9(1): 75-89. |
| Follicular B Cell | t(14; 18)(q32; q21) | bcl-2/Ig | Mohamed et al., 2001, |
| Lymphoma | | | Cancer Genet |
| | | | Cytogenet 126(1): 45- |
| | | | 51. |
| Multiple Myeloma | t(14; 16)(q32; q23) | WWOX (FRAI6D) | Krummel et al., 2000, |
| (MM) | | | Genomics 69(1): 37- |
| | | | 46; Bednarek et al., |
| | | | 2001, Cancer Res 61(22): |
| | | | 8068-73. |
| Malignant | der(12)t(12; 20)(q15; | | Sargent et al., 2001, |
| Melanoma (MM) | q11) | | Genes Chromosomes |
| | | | Cancer 32(1): 18-25. |
| Malignant | der(19)t(10; 19)(q23; | | Sargent et al., 2001, |
| Melanoma (MM) | q13) | | Genes Chromosomes |
| | | | Cancer 32(1): 18-25. |
| Malignant | der(12)t(12; 19)(q13; | | Sargent et al., 2001, |
| Melanoma (MM) | q13) | | Genes Chromosomes |
| | | | Cancer 32(1): 18-25. |
| MMSP Malignant | t(12; 22)(q13; q12) | EWS-ATF1 | Zucman et al., 1993, |
| Melanoma of Soft | | | Nat Genet 4(4): 341-5. |
| Parts | | | |
| Myelodysplastic | der(1; 18)(q10; q10) | | Wan et al., 2001, |
| Syndrome | | | Cancer Genet |
| (MDS); AML; | | | Cytogenet 128(1): 35- |
| Myeloproliferative | | | 8. |
| Disorder (MPD) | | | |
| MDS/AML | t(1; 3)(p36; q21) | | Mitelman et al., 1997, |
| | | | Nature Genetics Special |
| | | | Issue: 417-474. |
| Chronic | t(5; 12)(q33; p13) | TEL-PDGFβR | Tomasson et al., 1999, |
| Myelomonocytic | | | Blood 93(5): 1707-14. |
| Leukemia (CMML) | | | |
| Synovial Sarcoma | t(X; 18)(p11.2; | | Winnepenninckx et al., |
| | q11.2) | | 2001, Histopathol- |
| | | | ogy 38(2): 141-5. |
| Epitheloid Sarcoma | t(6; 8)(p25; q11.2) | | Feely et al., 2000, |
| | | | Cancer Genet |
| | | | Cytogenet 119(2): 155- |
| | | | 7. |
| Breast Cancer Cell | t(8; 11); t(12; 16); | | Kytola et al., 2000, |
| Lines | t(1; 16); t(15; 17) | | Genes Chromosomes |
| | | | Cancer 28: 308-317. |
| Neuroblastoma | der(11)t(11; 17) | | Panarello et al., 2000, |
| | (p15; q12)t(11; 17) | | Cancer Genet |
| | (q22; q12) | | Cytogenet 116(2): 124- |
| | | | 32. |
| Atypical CML | t(5; 10)(q33; q22) | H4/PDGFβR | Schwaller et al., 2001, |
| | | | Blood 97(12): 3910-18. |
| Atypical CML | t(5; 10)(q33; q21) | H4/PDGFβR | Kulkarni et al., 2000, |
| | | | Cancer Res 60(13): |
| | | | 3592-8. |
| Ewing Tumor | t(11; 22)(q24; q12) | EWS-FLI1 | Delattre et al., 1992, |
| | | | Nature 359(6391): 162- |
| | | | 5; May et al., 1993, |
| | | | Proc. Natl. Acad. Sci. |
| | | | USA 90: 5752-5756. |
| Ewing Tumor | t(21; 22)(q22; q12) | EWS-ERG | Sorensen et al., 1994, |
| | | | Nat Genet 6: 146-151. |
| Ewing Tumor | t(7; 22)(p22; q12) | EWS-ETV1 | Jeon et al., 1995, |
| | | | Oncogene 10: 1229- |
| | | | 1234. |
| Ewing Tumor | t(17; 22)(q12; q12) | EWS-E1AF | Kaneko et al., 1996, |
| | | | Genes Chromosomes |
| | | | Cancer 15: 115-121. |
| Ewing Tumor | t(2; 22)(q33; q12) | EWS-FEV | Peter et al., 1997, |
| | | | Oncogene 14: 1159- |
| | | | 1164. |
| (SS) Synovial | t(X; 18) | SYT-SSX1 | Clark et al., 1994, Nat |
| Sarcoma | (p11.2; q11.2) | | Genet 7: 502-508. |
| (SS) Synovial | t(X; 18) | SYT-SSX2 | Crew et al., 1995, |
| Sarcoma | (p11.2; q11.2) | | EMBO J 14: 2333- |
| | | | 2340; De Leeuw et al., |
| | | | 1995, Hum Mol Genet 4: |
| | | | 1097-1099. |
| (SS) Synovial | t(X; 18) | SYT-SSX4 | Skytting et al., 1999, J |
| Sarcoma | (p11.2; q11.2) | | Natl Cancer Inst 91: |
| | | | 974-975. |
| (SRCDT) Small | t(11; 22)(p13; q12) | EWS-WT1 | Ladanyi and Gerald, |
| Round Cell | | | 1994, Cancer Res 54: |
| Desmoplastic | | | 2837-2840; Willeke and |
| Tumors | | | Sturm, 2001, Seminars |
| | | | in Surgical Oncology 20: |
| | | | 294-303. |
| Extraskeletal | t(9; 22)(q22; q12) | EWS-TEC | Labelle et al., 1995, |
| Myxoid | | | Hum Mol Genet 4: |
| Chondrosarcoma | | | 2219-2226; Clark et al., |
| | | | 1996, Oncogene 12: |
| | | | 229-235. |
| Extraskeletal | t(9; 17)(q22; q11.2) | TAF68-TEC | Sjogren et al., 1999, |
| Myxoid | | | Cancer Res 59: 5064- |
| Chondrosarcoma | | | 5067; Panagopoulos et |
| | | | al., 1999, Oncogene 18: |
| | | | 7594-7598; Attwooll et |
| | | | al., 1999, Oncogene 18: |
| | | | 7599-7601. |
| Congenital | t(12; 15)(p13; q25) | ETV6-NTRK3 | Knezevich et al., 1998, |
| Fibrosarcoma | | | Nat Genet 18: 184-187. |
| Myxoid | t(12; 16)(q13; p11) | | Crozat et al., 1993, |
| Liposarcoma | | | Nature 363(6430): 640- |
| | | | 4. |
| Papillary Renal Cell | t(X; 1)(p11.2; q21.2) | PRCC/TFE3 | Meloni et al., 1993, |
| Carcinoma | | | Cancer Genet |
| | | | Cytogenet 65(1): 1-6; |
| | | | Shipley et al., 1995, |
| | | | Cytogenet Cell |
| | | | Genet 71(3): 280-4; |
| | | | Weterman et al., 1996, |
| | | | Cytogenet Cell |
| | | | Genet 75(1): 2-6; |
| | | | Weterman et al., 2001, |
| | | | Proc. Natl. Acad. |
| | | | Sci. 98(24): 13809-13813. |
| Dermofibrosarcoma | t(17; 22)(q22; q13) | PDGFβ-COL1A1 | Nishio et al., 2001, |
| Protuberans | | | Cancer Genet |
| | | | Cytogenet 129(2): 102- |
| | | | 6. |

The following sequences are provided to amplify the indicated partners of fused gene sequences involved malignant transformation:

| | |
|---|---|
| EWS 6f | CTCAGCCTGCTTATCCAGCC |
| EWS 7r | GCTATATTGACTTGGAGCTTGGC |
| EWS 3 | GTCAACCTCAATCTAGCACAGGG |
| FLI 3 | CTGTCGGAGAGCAGCTCCAG |
| ERG 3 | CTGTCCGACAGGAGCTCAG |
| FEV 2 | GAAACTGCCACAGCTGGATC |
| ETV1.1 | TAAATTCCATGCCTCGACCAG |
| E1AF.1 | AACTCCATTCCCCGGCC |
| Pax3.1 | TCCAACCCCATGAACCCC |
| Pax7.1 | CAACCACATGAACCCGGTC |
| FKHR1.2 | GCCATTTGGAAAACTGTGATCC |
| EWS 12 | AGCCAACAGAGCAGCAGCTAC |
| WT1.3 | TGAGTCCTGGTGTGGGTCTTC |
| SYT.2 | TACCCAGGGCAGCAAGGTT |
| SSXc.3 | ATCGTTTTGTGGGCCAGATG |
| ETV6.1 | CCCATCAACCTCTCTCATCGG |
| NTRK3.1 | GGCTCCCTCACCCAGTTCTC |
| ALK.1 | AGGTCACTGATGGAGGAGGTCTT |
| NPM.1 | CTTGGGGGCTTTGAAATAACAC |
| TM30.1 | CCGTGCTGAGTTTGCTGAGAG |
| TFG.1 | AGAACCAGGACCTTCCACCAATA |
| ATIC.1 | AGGCATTCACTCATACGGCAC |
| EWS.15 | CCCACTAGTTACCCACCCCAAA |
| TAF68.1 | AGCAAAACATGGAATCATCAGGA |
| TEC.3 | TACACGCAGGAAGGCTTGAGTT |
| ATF1.1 | TGTAAGGCTCCATTTGGGGC |
| EWS S2 | CTCCTACCAGCTATTCCTCTACACAGCCGACT |
| RMS S1 | ATGCTCAATCCAGAGGGTGGCAAGAG |
| WT1 | TCTCGTTCAGACCAGCTCAAAAGACACCA |
| SYNO S1 | ATCATGCCCAAGAAGCCAGCAGAGG |
| FC1 S1 | CTCCCCGCCTGAAGAGCACGC |
| ALK S1 | CAAGCTCCGCACCTCGACCATCA |
| TEC S1 | ACCTTGGCAGCACTGAGATCACGGC |

The following primer pairs may be used in the methods of the invention to isolate and further characterize the following gene fusions:

| | 5′ primer | 3′ primer |
|---|---|---|
| EWS-FL1 fusions | EWS 3 | FL1.3 |
| EWS-ERG | EWS 3 | ERG 3 |
| EWS-ETV1 | EWS 3 | ETV1.1 |
| EWS-E1AF | EWS 3 | E1AF.1 |
| EWS-FEV | EWS 3 | FEV 2 |
| PAX3-FKHR | Pax3.1 | FKHR1.2 |
| PAX7-FKHR | Pax7.1 | FKHR1.2 |
| SYT-SSX1 | SYT.2 | SSXc.3 |
| SYT-SSX2 | SYT.2 | SSXc.3 |
| SYT-SSX4 | SYT.2 | SSXc.3 |
| EWS-WT1 | EWS 12 | WT1.3 |
| EWS-TEC | EWS 15 | TEC S1 |
| TAF68-TEC | TAF68.1 | TEC.3 |
| EWS-ATF1 | EWS 3 | ATF1.1 |
| ETV6-NTRK3 | ETV6.1 | NTRK3.1 |
| NPM-ALK | NPM.1 | ALK.1 |
| TPM3-ALK | TM30.1 | ALK.1 |
| TFG-ALK | TFG.1 | ALK.1 |
| ATIC-ALK | TAIC.1 | ALK.1 |

To isolate gene fusions involving the B cell receptor gene which are frequently associated with myeloproliferative disorders, the following primers may be used in the methods of the invention:

| | | |
|---|---|---|
| BCR | (intron 4, forward) | GGGCCAAGGAGACCAGTGAGT |
| BCR | (intron 4, reverse) | AACAGCCAGCCTGAGGTAGGG |
| FGFR1 | (exons 5-6 forward) | ACATCGAGGTGAATGGGAGCAA |
| FGFR1 | (exon 12, reverse) | TTGGAGGAGAGCTGCTCCTCT |
| BCR | (exon 1, forward) | CCCCGGAGTTTTGAGGATTG |
| ABL | (exon 3) | TGGCGTGATGTAGTTGCTTGG |

The disclosures of each and every patent, patent application, and publication cited herein are hereby incorporated herein by reference in their entirety.

While this invention has been disclosed with reference to specific embodiments, other embodiments and variations of this invention may be devised by those of skill in the art without departing from the true spirit and scope of the invention. The appended claims are intended to be construed to include all such embodiments and equivalent variations.

What is claimed is:

1. A method of amplifying an unknown region which flanks a known region of a cancer-associated DNA sequence, the method comprising
(a) providing a template polynucleotide comprising a sense strand which comprises said known region and said unknown region, wherein said unknown region is nearer the 3′-end of said sense strand than is said known region, wherein said known region comprises a first portion and a second portion, and wherein said first portion is nearer said unknown region than is said second portion;
(b) ligating a loop-forming oligonucleotide to the 3′-end of said sense strand, wherein said loop-forming oligonucleotide is complementary to said first portion;
(c) annealing said loop-forming oligonucleotide with said first portion to generate a panhandle structure;
(d) subjecting said panhandle structure to extension, whereby a third region complementary to said second portion is generated at the free end of said loop-forming oligonucleotide; and
(e) subjecting said panhandle structure to PCR in the presence of a first primer homologous with said second portion, whereby said unknown region is amplified.

2. The method of claim 1, wherein said cancer-associated DNA sequence comprises a gene partner set forth in Table 1.

3. The method of claim 2, wherein said known region comprises a portion of the breakpoint cluster region of a gene set forth in Table 1.

4. The method of claim 1, wherein said cancer-associated DNA sequence comprises ATF1.

5. The method of claim 1, wherein said cancer-associated DNA sequence comprises BCR.

6. The method of claim 1, wherein said first primer has a nucleotide sequence selected from the group consisting of

| | |
|---|---|
| EWS 6f | CTCAGCCTGCTTATCCAGCC; |
| EWS 7r | GCTATATTGACTTGGAGCTTGGC; |
| EWS 3 | GTCAACCTCAATCTAGCACAGGG; |
| FLI 3 | CTGTCGGAGAGCAGCTCCAG; |
| ERG 3 | CTGTCCGACAGGAGCTCAG; |
| FEV 2 | GAAACTGCCACAGCTGGATC; |
| ETV1.1 | TAAATTCCATGCCTCGACCAG; |
| E1AF.1 | AACTCCATTCCCCGGCC; |
| Pax3.1 | TCCAACCCCATGAACCCC; |
| Pax7.1 | CAACCACATGAACCCGGTC; |
| FKHR1.2 | GCCATTTGGAAAACTGTGATCC; |
| EWS 12 | AGCCAACAGAGCAGCAGCTAC; |
| WT1.3 | TGAGTCCTGGTGTGGGTCTTC; |
| SYT.2 | TACCCAGGGCAGCAAGGTT; |
| SSXc.3 | ATCGTTTTGTGGGCCAGATG; |
| ETV6.1 | CCCATCAACCTCTCTCATCGG; |
| NTRK3.1 | GGCTCCCTCACCCAGTTCTC; |
| ALK.1 | AGGTCACTGATGGAGGAGGTCTT; |
| NPM.1 | CTTGGGGGCTTTGAAATAACAC; |
| TM30.1 | CCGTGCTGAGTTTGCTGAGAG; |
| TFG.1 | AGAACCAGGACCTTCCACCAATA; |
| ATIC.1 | AGGCATTCACTCATACGGCAC; |
| EWS.15 | CCCACTAGTTACCCACCCCAAA; |
| TAF68.1 | AGCAAAACATGGAATCATCAGGA; |
| TEC.3 | TACACGCAGGAAGGCTTGAGTT; |
| ATF1.1 | TGTAAGGCTCCATTTGGGGC; |
| EWS S2 | CTCCTACCAGCTATTCCTCTACACAGCCGACT; |
| RMS S1 | ATGCTCAATCCAGAGGGTGGCAAGAG; |
| WT1 | TCTCGTTCAGACCAGCTCAAAAGACACCA; |
| SYNO S1 | ATCATGCCCAAGAAGCCAGCAGAGG; |
| FC1 S1 | CTCCCCGCCTGAAGAGCACGC; |
| ALK S1 | CAAGCTCCGCACCTCGACCATCA; |
| TEC S1 | ACCTTGGCAGCACTGAGATCACGGC; |
| BCR | GGGCCAAGGAGACCAGTGAGT; |
| (intron 4, | |
| forward) | |
| BCR | AACAGCCAGCCTGAGGTAGGG; |
| (intron 4, | |
| reverse) | |
| FGFR1 | ACATCGAGGTGAATGGGAGCAA; |
| (exons 5-6 | |
| forward) | |
| FGFR1 | TTGGAGGAGAGCTGCTCCTCT; |
| (exon 12, | |
| reverse) | |
| BCR | CCCCGGAGTTTTGAGGATTG; and |
| (exon 1, | |
| forward) | |
| ABL (exon 3) | TGGCGTGATGTAGTTGCTTGG. |
